(19) United States
(12) Patent Application Publication
(10) Pub. No.: US 2026/0317304 A1
(43) Pub. Date: Oct. 15, 2026

(54) TEAD INHIBITORS AND METHODS OF USE

(71) Applicant: Sporos BioDiscovery, Inc., Houston, TX (US)

(72) Inventors: Florian Muller, Houston, TX (US); Andrew Morley, Cheshire (GB); Sharon Shacham, Sunny Isle, FL (US)

(21) Appl. No.: 18/996,871

(22) PCT Filed: Jul. 17, 2023
(86) PCT No.: PCT/US2023/070353

§ 371 Date: Jan. 17, 2025

Related U.S. Application Data
(60) Provisional application No. 63/369,036, filed on Jul. 21, 2022.

(51) Int. Cl. C07D 403/10; A61K 31/4015; A61K 31/4178; A61K 31/422; A61K 31/4245; A61K 31/497; A61K 31/501; A61P 35/00; C07D 207/26; C07D 233/64; C07D 233/76; C07D 413/10

(52) U.S. Cl. CPC C07D 403/10; A61K 31/4015; A61K 31/4178; A61K 31/422; A61K 31/4245; A61K 31/497; A61K 31/501; A61P 35/00; C07D 207/26; C07D 233/64; C07D 233/76; C07D 413/10

(57) ABSTRACT

The present disclosure provides, in part, compounds of formula (I): wherein the variables are as defined herein, pharmaceutical compositions comprising the compounds, and methods of using the compounds to treat physiological disorders, such as proliferative disorders, mediated by TEA domain transcription factors.

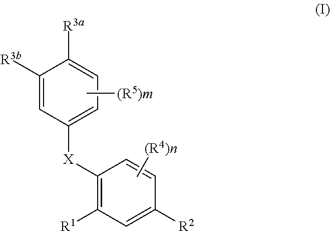

### CROSS REFERENCE TO RELATED APPLICATIONS

This application claims priority to and the benefit of U.S. Provisional Application No. 63/369,036, filed Jul. 21, 2022, the contents of which are hereby incorporated by reference in their entirety.

### SUMMARY OF THE DISCLOSURE

Disclosed herein, in certain embodiments, are compounds of formula (I), formula (I′), formula (I-1), or formula (I-1′), e.g., compounds of formula (Ia), formula (Ia′), formula (Ib), formula (Ib′), formula (Ic), formula (Ic′), formula (Id), and/or formula (Ie), or pharmaceutically acceptable salts thereof. Additionally, disclosed herein, in certain embodiments, are compositions comprising a compound of formula (I), formula (I′), formula (I-1), or formula (I-1′), e.g., a compound of formula (Ia), formula (Ia′), formula (Ib), formula (Ib′), formula (Ic), formula (Ic′), formula (Id), and/or formula (Ie), or a pharmaceutically acceptable salt thereof, and a pharmaceutically acceptable excipient. Further disclosed herein, in certain embodiments, are methods of inhibiting abnormal transcriptional enhanced associate (TEA) domain transcription factor (TEAD) transcriptional complex activity and thus treating certain diseases or disorders such as cancer characterized by abnormal TEAD transcriptional complex activity (e.g., hyperactivation).

Disclosed herein, in certain embodiments, are compounds of formula (I):

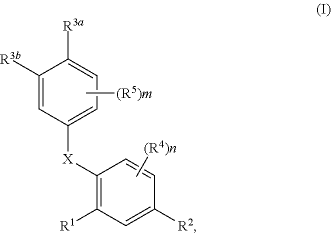

or pharmaceutically acceptable salts thereof, wherein the variables are as defined herein.

Also, disclosed herein, in certain embodiments, are compounds of formula (I′):

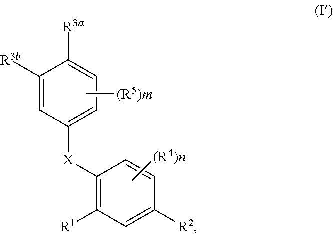

or pharmaceutically acceptable salts thereof, wherein the variables are as defined herein.

Also, disclosed herein, in certain embodiments, are compounds of formula (I-1):

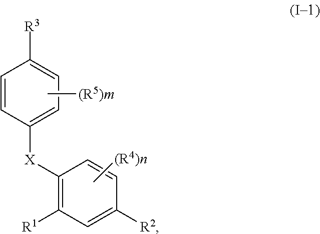

or pharmaceutically acceptable salts thereof, wherein the variables are as defined herein.

Also, disclosed herein, in certain embodiments, are compounds of formula (I-1′):

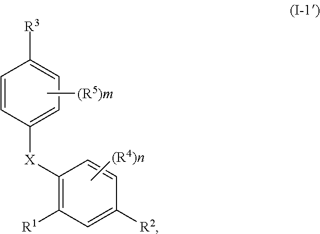

or pharmaceutically acceptable salts thereof, wherein the variables are as defined herein.

Also, disclosed herein, in certain embodiments, are compounds of formula (Ia):

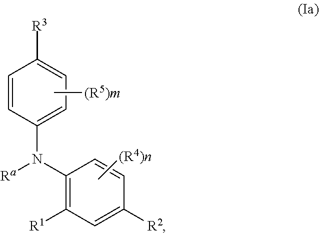

or pharmaceutically acceptable salts thereof, wherein the variables are as defined herein.

Also, disclosed herein, in certain embodiments, are compounds of formula (Ia′):

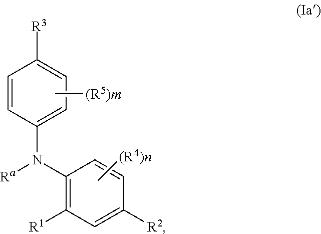

or pharmaceutically acceptable salts thereof, wherein the variables are as defined herein.

Also, disclosed herein, in certain embodiments, are compounds of formula (Ib):

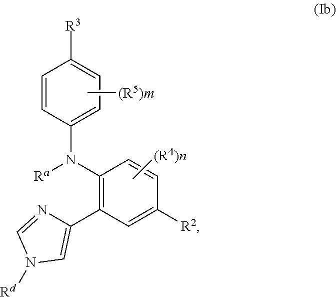

or pharmaceutically acceptable salts thereof, wherein the variables are as defined herein.

Also, disclosed herein, in certain embodiments, are compounds of formula (Ib′):

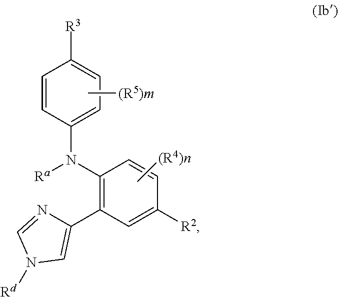

or pharmaceutically acceptable salts thereof, wherein the variables are as defined herein.

Also, disclosed herein, in certain embodiments, are compounds of formula (Ic):

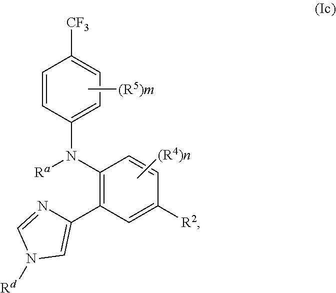

or pharmaceutically acceptable salts thereof, wherein the variables are as defined herein.

Also, disclosed herein, in certain embodiments, are compounds of formula (Ic′):

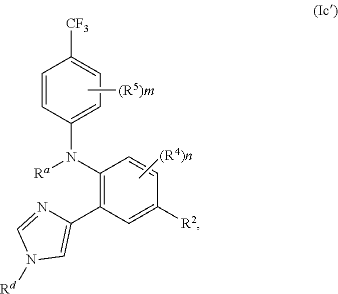

or pharmaceutically acceptable salts thereof, wherein the variables are as defined herein.

Also, disclosed herein, in certain embodiments, are compounds of formula (Id):

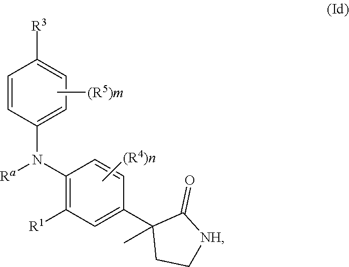

or pharmaceutically acceptable salts thereof, wherein the variables are as defined herein.

Also, disclosed herein, in certain embodiments, are compounds of formula (Ie):

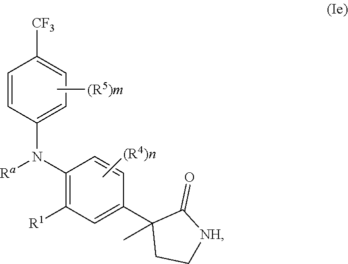

or pharmaceutically acceptable salts thereof, wherein the variables are as defined herein.

In some embodiments, the compounds of formula (I), formula (I′), formula (I-1), formula (I-1′), formula (Ia), formula (Ia′), formula (Ib), formula (Ib′), formula (Ic), formula (Ic′), formula (Id), or formula (Ie) are selected from the compounds of Table 1 or a pharmaceutically acceptable salt thereof.

Also provided herein, in certain embodiments, are pharmaceutical compositions comprising a compound disclosed herein or a pharmaceutically acceptable salt thereof, and a pharmaceutically acceptable excipient.

Further disclosed herein, in certain embodiments, are methods of treating a disease or condition mediated by TEAD activity (e.g., disregulation) in a subject in need thereof, comprising administering to the subject a therapeutically effective amount of a compound or pharmaceutically acceptable salt described herein or a pharmaceutical composition disclosed herein. In some embodiments, the disease or condition is a cancer characterized by abnormal TEAD transcriptional complex activity (e.g., hyperactivation).

### BRIEF DESCRIPTION OF THE DRAWINGS

The disclosure can be more completely understood with reference to the following drawings.

FIG. 1 is a diagram showing the domain architecture of human TEAD1, TEAD2, TEAD3, and TEAD4. The percent values represent the identity of the N-terminal DNA binding domain (DNA-BD) and C-terminal YAP/TAZ binding domain (YAP/TAZ-BD) of TEAD2-4 compared to the respective binding domains of TEAD1. Post translation modifications including palmitoylation and phosphorylation as well as the p38 binding D domain of DNA-BD are also shown.

FIG. 2A is a diagram showing the upstream signaling and downstream transcriptional outputs of TEAD in cancer biology, which regulate critical functions in tumorigenesis, stem cell maintenance, cancer immunology, and metabolism, as well as formation of signaling feedback loops. Oncogenic signal transduction pathways include EGFR signaling, TGFβ signaling, WNt signaling, GPCR signaling, and cancer genes (indicated with *), such as KRAS, BRAF, LKB1, APC, and GNAQ/11.

FIG. 2B is a diagram showing the role of TEAD in multiple stages of tumorigenesis.

FIG. 3A is a graph showing the pre- and 7 day post-treatment tumor volume in individual mice bearing MSTO-211H tumors treated with vehicle twice daily, 3 mg/kg Compound 1-1 twice daily, or 12.5 mg/kg Compound 5 twice daily.

FIG. 3B is a graph showing the mean change in MSTO-211H tumor volume in the treatment groups described in FIG. 3A.

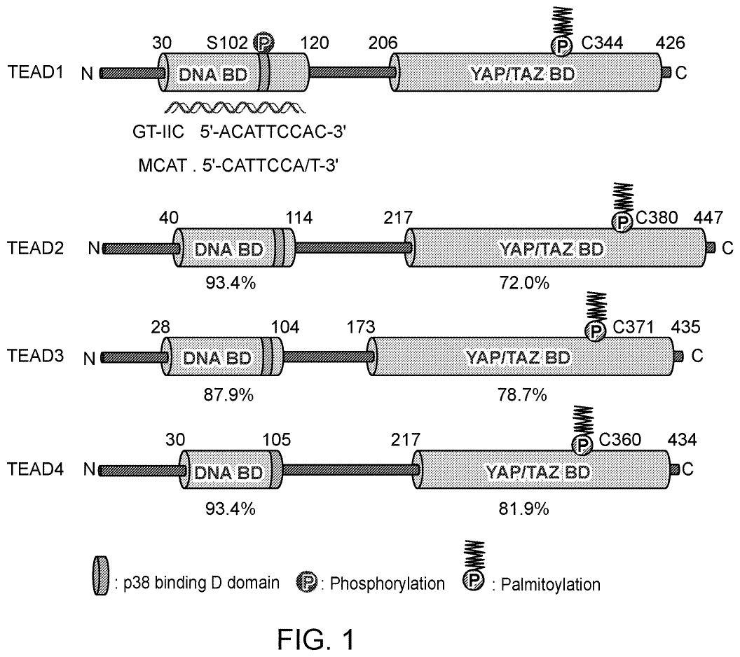

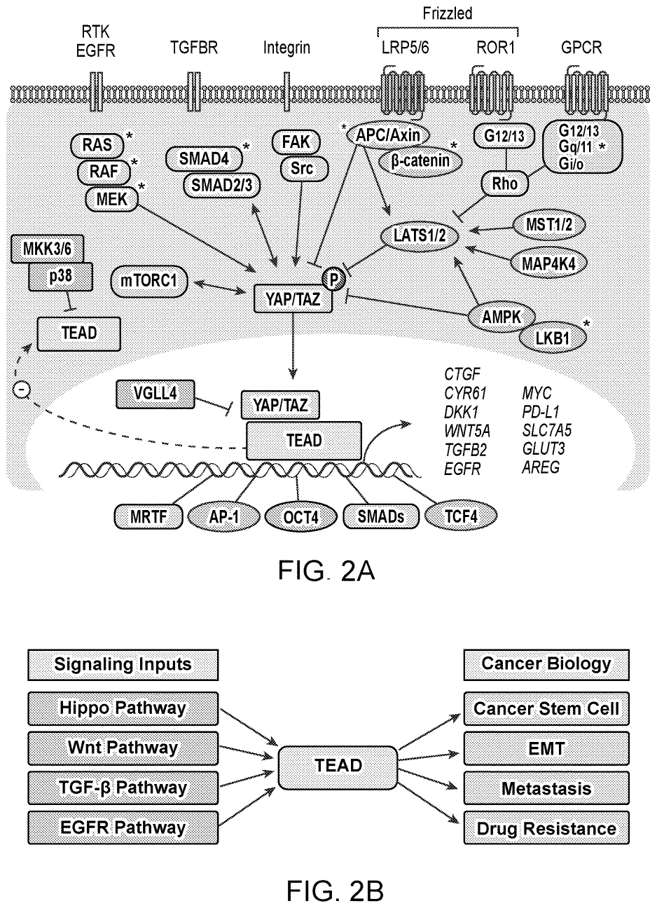

### DETAILED DESCRIPTION

Provided herein, in certain embodiments, are compounds and compositions, e.g., compounds of formula (I), formula (I′), formula (I-1), or formula (I-1′), e.g., compounds of formula (Ia), formula (Ia′), formula (Ib), formula (Ib′), formula (Ic), formula (Ic′), formula (Id), and/or formula (Ie), or pharmaceutically acceptable salts thereof. Further provided herein, in certain embodiments, are compositions comprising a compound of formula (I), formula (I′), formula (I-1), or formula (I-1′), e.g., a compound of formula (Ia), formula (Ia′), formula (Ib), formula (Ib′), formula (Ic), formula (Ic′), formula (Id), and/or formula (Ie), or a pharmaceutically acceptable salt thereof, and a pharmaceutically acceptable excipient. Also provided herein, in certain embodiments, are methods of use of compounds and compositions disclosed herein. The contemplated compounds and compositions disclosed herein are inhibitors of transcriptional enhancer factor domain (TEAD) activity and thus are useful in methods of treating certain diseases or disorders such as cancer characterized by abnormal TEAD transcriptional complex activity (e.g., hyperactivation).

### Certain Terminology

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

The articles “a” and “an” are used herein to refer to one or to more than one (i.e. at least one) of the grammatical objects of the article.

### Chemical Definitions

Definitions of specific functional groups and chemical terms are described in more detail below.

In some embodiments, compounds described herein comprise one or more asymmetric centers, and thus exist in various isomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. The disclosure additionally encompasses compounds described herein as individual isomers substantially free of other isomers, and alternatively, as mixtures of various isomers.

In some embodiments, compounds described herein also comprise one or more isotopic substitutions. For example, in some embodiments, H is in any isotopic form, including ¹H, ²H (D or deuterium), and ³H (T or tritium); C is any isotopic form, including ¹²C, ¹³C, and ¹⁴C; O is in any isotopic form, including ¹⁶O and ¹⁸O; F is in any isotopic form, including ¹⁸F and ¹⁹F; and the like.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁-C₂₀ alkyl”). In some embodiments, an alkyl group has 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). Examples of C₁-C₆ alkyl groups include methyl, ethyl, propyl, isopropyl, butyl, isobutyl, pentyl, hexyl, and the like.

As used herein, “alkylene,” refers to a divalent radical of an alkyl group. When a range or number of carbons is provided for a particular “alkylene” group, it is understood that the range or number refers to the range or number of carbons in the linear carbon divalent chain. In some embodiments, “alkylene” group is substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In some embodiments, in heteroaryl groups that contain one or more nitrogen atoms, the point of attachment is a carbon or nitrogen atom, as valency permits.

In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

The term “cycloalkyl” refers to a monovalent saturated cyclic, bicyclic, or bridged cyclic (e.g., adamantyl) hydrocarbon group of 3-12, 3-10, 3-8, 4-8, or 4-6 carbons, referred to herein, e.g., as “C₃-C₁₀cycloalkyl,” derived from a cycloalkane. Exemplary cycloalkyl groups include, but are not limited to, cyclohexanes, cyclopentanes, cyclobutanes and cyclopropanes.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, and sulfur (“3-10 membered heterocyclyl”). In some embodiments, in heterocyclyl groups that contain one or more nitrogen atoms, the point of attachment is a carbon or nitrogen atom, as valency permits. In some embodiments, a heterocyclyl group is either be monocyclic (“monocyclic heterocyclyl”) or a fused, bridged or spiro ring system such as a bicyclic system (“bicyclic heterocyclyl”), and is saturated or is partially unsaturated. In some embodiments, heterocyclyl bicyclic ring systems include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more phenyl or heteroaryl groups, wherein the point of attachment is on the heterocyclyl ring, and in such instances, the number of ring members continue to designate the number of ring members in the heterocyclyl ring system. The terms “heterocycle,” “heterocyclyl,” “heterocyclyl ring,” “heterocyclic group,” “heterocyclic moiety,” and “heterocyclic radical,” are used interchangeably.

In some embodiments, a heterocyclyl group is a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In some embodiments, a heterocyclyl group is a 3-7 membered non-aromatic ring system having ring carbon atoms and 1-4 ring heteroatoms, wherein each heteroatom is independently selected from nitrogen, oxygen, and sulfur (“3-7 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

“Hetero” when used to describe a compound or a group present on a compound means that one or more carbon atoms in the compound or group have been replaced by a nitrogen, oxygen, or sulfur heteroatom. In some embodiments, hetero is applied to any of the hydrocarbyl groups described above such as alkyl, e.g., heteroalkyl; cycloalkyl, e.g., heterocyclyl; aryl, e.g., heteroaryl; and the like having from 1 to 5, and particularly from 1 to 3 heteroatoms.

The terms “halo” and “halogen” as used herein refer to an atom selected from fluorine (fluoro, —F), chlorine (chloro, —Cl), bromine (bromo, —Br), and iodine (iodo, —I). In certain embodiments, the halo group is either fluoro or chloro.

The term “haloalkyl” includes mono, poly, and perhaloalkyl groups substituted with one or more halogen atoms where the halogens are independently selected from fluorine, chlorine, bromine, and iodine.

The term “oxo” as used herein refers to ═O.

The term “hydroxyl” as used herein refers to —OH.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms are substituted or unsubstituted as valency permits, and include primary, secondary, tertiary, and quaternary nitrogen atoms.

These and other exemplary substituents are described in more detail in the Detailed Description, Examples, and Claims. The disclosure is not intended to be limited in any manner by the above exemplary listing of substituents.

### Other Definitions

As used herein, “pharmaceutically acceptable excipient” refers to any substance in a pharmaceutical formulation other than the active pharmaceutical ingredient(s). Exemplary pharmaceutical excipients include those that aid the manufacturing process; protect, support or enhance stability; increase bioavailability; or increase patient acceptability. They may also assist in product identification or enhance the overall safety or function of the product during storage or use.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” “individual” and “subject” are used interchangeably herein. None of the terms require the supervision of medical personnel.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition.

As used herein, and unless otherwise specified, a “therapeutically effective amount” or an “effective amount” of a compound is an amount sufficient to provide a therapeutic benefit in the treatment of a disease, disorder or condition, or to delay or minimize one or more symptoms associated with the disease, disorder or condition. A therapeutically effective amount or an effective amount of a compound means an amount of therapeutic agent, alone or in combination with other therapies, which provides a therapeutic benefit in the treatment of the disease, disorder or condition. In some embodiments, the term “therapeutically effective amount” or “effective amount” encompasses an amount that improves overall therapy, reduces or avoids symptoms or causes of disease or condition, or enhances the therapeutic efficacy of another therapeutic agent.

### Compounds

Disclosed herein, in certain embodiments, are compounds of formula (I):

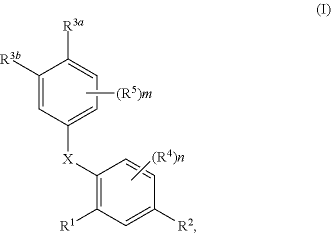

or pharmaceutically acceptable salts thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl), wherein the 3-10 membered heterocyclyl has one, two, three, or four heteroatoms each independently selected from the group consisting of N, O, and S and is optionally substituted, wherein the C₁-C₄alkylene is optionally substituted;
one of R^{3a} and R^{3b} is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted; and the other one of R^{3a} and R^{3b} is selected from the group consisting of hydrogen, halogen, and —C₁-C₆ alkyl;
each R⁴ is independently selected from halogen and —C₁-C₆ alkyl;
each R⁵ is independently selected from halogen and —C₁-C₆ alkyl;
m is selected from the group consisting of 0, 1, 2, and 3;
n is selected from the group consisting of 0, 1, 2, and 3; and
R^{a} is selected from hydrogen and —C₁-C₆ alkyl.

Also disclosed herein, in certain embodiments, are compounds of formula (I′):

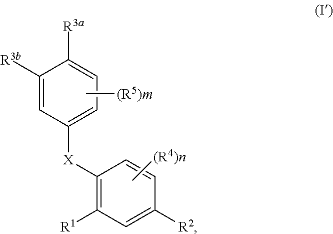

or pharmaceutically acceptable salts thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl), wherein the 3-10 membered heterocyclyl contains at least 2 carbon atoms and one, two, three, or four heteroatoms and is optionally substituted, wherein the heteroatoms are nitrogen, and wherein the C₁-C₄alkylene is optionally substituted;
one of R^{3a} and R^{3b} is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted; and the other one of R^{3a} and R^{3b} is selected from the group consisting of hydrogen, halogen, and —C₁-C₆ alkyl;
each R⁴ is independently selected from halogen and —C₁-C₆ alkyl;
each R⁵ is independently selected from halogen and —C₁-C₆ alkyl;
m is selected from the group consisting of 0, 1, 2, and 3;
n is selected from the group consisting of 0, 1, 2, and 3; and
R^{a} is selected from hydrogen and —C₁-C₆ alkyl.

Also disclosed herein, in certain embodiments are compounds of formula (I-1):

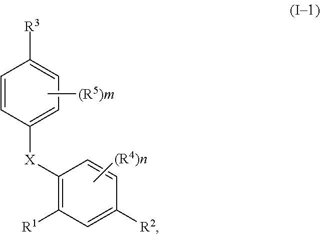

or pharmaceutically acceptable salts thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl); wherein the 3-10 membered heterocyclyl has one, two, three, or four heteroatoms each independently selected from the group consisting of N, O, and S, and is optionally substituted, wherein the C₁-C₄alkylene is optionally substituted;
R³ is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted;
each R⁴ is independently selected from halogen and —C₁-C₆ alkyl;
each R⁵ is independently selected from halogen and —C₁-C₆ alkyl;
m is selected from the group consisting of 0, 1, 2, 3, and 4;
n is selected from the group consisting of 0, 1, 2, and 3; and
R^{a} is selected from hydrogen and —C₁-C₆ alkyl.

Also disclosed herein, in certain embodiments are compounds of formula (I-1):

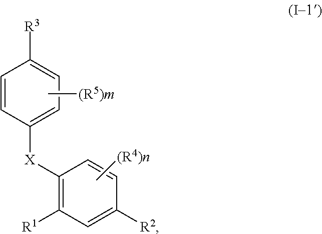

or pharmaceutically acceptable salts thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl); wherein the 3-10 membered heterocyclyl contains at least 2 carbon atoms and one, two, three, or four heteroatoms and is optionally substituted, wherein the heteroatoms are nitrogen, and wherein the C₁-C₄alkylene is optionally substituted;
R³ is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted;
each R⁴ is independently selected from halogen and —C₁-C₆ alkyl;
each R⁵ is independently selected from halogen and —C₁-C₆ alkyl;
m is selected from the group consisting of 0, 1, 2, 3, and 4;
n is selected from the group consisting of 0, 1, 2, and 3; and
R^{a} is selected from hydrogen and —C₁-C₆ alkyl.

In some embodiments, X is NR^{a}.

In some embodiments, the compounds are compounds of formula (Ia):

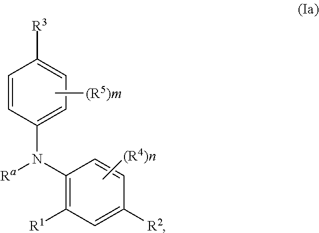

or pharmaceutically acceptable salts thereof, wherein:
R³ is —C₁-C₆ alkyl or —C₁-C₆ haloalkyl; and
the rest of the variables are as defined in formula (I).

In some embodiments, the compounds are compounds of formula (Ia′):

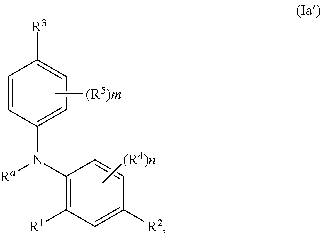

or pharmaceutically acceptable salts thereof, wherein:
R³ is —C₁-C₆ alkyl or —C₁-C₆ haloalkyl; and
the rest of the variables are as defined in formula (I′).

In some embodiments, R¹ is 5-6 membered heteroaryl that is optionally substituted.

In some embodiments, R¹ is 5-6 membered heteroaryl that is optionally substituted with 1, 2, 3, or 4 substituents each independently selected from the group consisting of C₁-C₆ alkyl, halogen, C₁-C₆ haloalkyl, —O—(C₁-C₆ alkyl), —O—(C₁-C₆ haloalkyl), and —N(R^{b})₂, wherein each R^{b} is independently selected from hydrogen and C₁-C₆ alkyl.

In some embodiments, R¹ is 5-6 membered heteroaryl selected from the group consisting of oxadiazole, imidazole, pyrazole, oxazole, triazole, tetrazole, pyridine, pyrazine, and pyridazine, wherein the 5-6 membered heteroaryl is optionally substituted with 1, 2, 3, or 4 substituents each independently selected from the group consisting of C₁-C₆ alkyl, halogen, C₁-C₆ haloalkyl, —O—(C₁-C₆ alkyl), —O—(C₁-C₆ haloalkyl), and —N(R^{b})₂, wherein each R^{b} is independently selected from hydrogen and C₁-C₆ alkyl.

In some embodiments, R¹ is 5-6 membered heteroaryl that is optionally substituted with 1, 2, 3, or 4 substituents each independently selected from halogen and —C₁-C₆ alkyl.

In some embodiments, R¹ is 5-6 membered heteroaryl selected from the group consisting of oxadiazole, imidazole, pyrazole, oxazole, triazole, tetrazole, pyridine, pyrazine, and pyridazine, wherein the 5-6 membered heteroaryl is optionally substituted with C₁-C₆ alkyl.

In some embodiments, R¹ is imidazole optionally substituted with methyl.

In some embodiments, R¹ is oxazole.

In some embodiments, R¹ is tetrazole optionally substituted with methyl.

In some embodiments, R¹ is oxadiazole, e.g., 1,3,4-oxadiazole, optionally substituted with methyl.

In some embodiments, the compounds are compounds of formula (Ib):

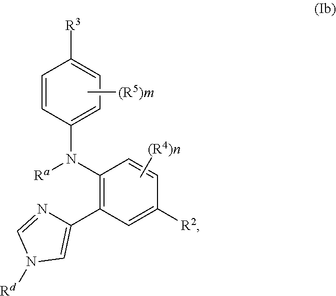

or pharmaceutically acceptable salts thereof, wherein:
R^{d} is selected from hydrogen and —C₁-C₆ alkyl; and
the rest of the variables are as defined in formula (Ia).

In some embodiments, the compounds are compounds of formula (Ib′):

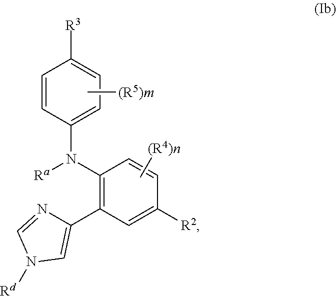

or pharmaceutically acceptable salts thereof, wherein:
R^{d} is selected from hydrogen and —C₁-C₆ alkyl; and
the rest of the variables are as defined in formula (Ia′).

In some embodiments, the compounds are compounds of formula (Ic):

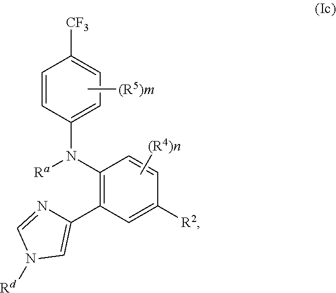

or pharmaceutically acceptable salts thereof, wherein the variables are as defined in formula (Ib).

In some embodiments, the compounds are compounds of formula (Ic′):

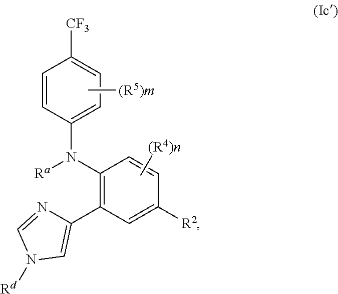

or pharmaceutically acceptable salts thereof, wherein the variables are as defined in formula (Ib′).

In some embodiments, R² is 3-7 membered monocyclic heterocyclyl or —(C₁-C₄alkylene)-(3-7 membered monocyclic heterocyclyl), wherein the 3-7 membered monocyclic heterocyclyl has one, two, three, or four heteroatoms each independently selected from N, O, and S, wherein the 3-7 membered monocyclic heterocyclyl is optionally substituted, and wherein the C₁-C₄alkylene is optionally substituted (e.g., optionally substituted with C₁₋₆alkyl, C₁₋₆haloalkyl, or two geminal hydrogens on a carbon of the C₁₋₄alkylene can be taken together with the carbon atom to which they are attached to form a 3-7 membered cycloalkyl ring).

In some embodiments, R² is 3-7 membered monocyclic heterocyclyl or —(C₁-C₄alkylene)-(3-7 membered monocyclic heterocyclyl), wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted, and wherein the C₁-C₄alkylene is optionally substituted (e.g., optionally substituted with C₁₋₆alkyl, C₁₋₆haloalkyl, or two geminal hydrogens on a carbon of the C₁₋₄ alkylene can be taken together with the carbon atom to which they are attached to form a 3-7 membered cycloalkyl ring).

In some embodiments, R² is 3-7 membered monocyclic heterocyclyl or —(C₁-C₄alkylene)-(3-7 membered monocyclic heterocyclyl), wherein the 3-7 membered monocyclic heterocyclyl has one, two, three, or four heteroatoms each independently selected from N, O, and S, and is optionally substituted with 1, 2, 3, or 4 substituents each independently selected from the group consisting of C₁-C₆ alkyl, oxo, halogen, C₁-C₆ haloalkyl, —O—(C₁-C₆ alkyl), —O—(C₁-C₆ haloalkyl), —N(R^{b})₂, —C(O)OR^{g}, —C(O)N(R^{f})₂, —S(O)₂N(R^{f})₂, —OC(O)N(R^{f})₂, —NR^{f}C(O)N(R^{f})₂, —S(O)ᵣ—R^{f}, wherein each of R^{b}, R^{g}, and R^{f} is independently selected from hydrogen and C₁-C₆ alkyl, wherein each r is 1 or 2, and wherein each R^{f} is independently selected from C₁₋₆alkyl and phenyl.

In some embodiments, R² is 3-7 membered monocyclic heterocyclyl or —(C₁-C₄alkylene)-(3-7 membered monocyclic heterocyclyl), wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted with 1, 2, 3, or 4 substituents each independently selected from the group consisting of C₁-C₆ alkyl, oxo, hydroxyl, halogen, C₁-C₆ haloalkyl, —O—(C₁-C₆ alkyl), —O—(C₁-C₆ haloalkyl), —N(R^{b})₂, —C(O)OR^{g}, —C(O)N(R^{f})₂, —S(O)₂N(R^{f})₂, —OC(O)N(R^{f})₂, —NR^{f}C(O)N(R^{f})₂, and —S(O)ᵣ—R^{f}, wherein each of R^{b}, R^{g}, and R^{f} is independently selected from hydrogen and C₁-C₆ alkyl, wherein each r is 1 or 2, and wherein each R^{f} is independently selected from C₁₋₆alkyl and phenyl.

In some embodiments, R² is 3-7 membered monocyclic heterocyclyl, wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted with 1, 2, 3, or 4 substituents each independently selected from the group consisting of halogen, —C₁-C₆ alkyl, oxo, and hydroxyl.

In some embodiments, R² is 5-membered heterocyclyl having one or two nitrogen atoms, wherein the 5-membered heterocyclyl is optionally substituted with 1, 2, or 3 substituents each independently selected from C₁-C₆ alkyl and oxo.

In some embodiments, R² is 5-membered heterocyclyl having one or two heteroatoms, wherein the heteroatoms are nitrogen atoms, and wherein the 5-membered heterocyclyl is optionally substituted with 1, 2, or 3 substituents each independently selected from C₁-C₆ alkyl, oxo, and hydroxyl.

In some embodiments, R² is —(C₁-C₄alkylene)-(5-membered heterocyclyl having one or two nitrogen atoms), wherein the 5-membered heterocyclyl is optionally substituted with 1, 2, or 3 substituents each independently selected from C₁-C₆ alkyl and oxo.

In some embodiments, R² is —(C₁-C₄alkylene)-(5-membered heterocyclyl having one or two heteroatoms), wherein the heteroatoms are nitrogen atoms, and wherein the 5-membered heterocyclyl is optionally substituted with 1, 2, or 3 substituents each independently selected from C₁-C₆ alkyl, oxo, and hydroxyl.

In some embodiments, R² is:

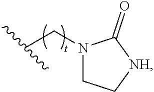

wherein t is selected from the group consisting of 0, 1, 2, and 3.

In some embodiments, R² is:

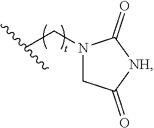

wherein t is selected from the group consisting of 0, 1, 2, and 3.

In some embodiments, t is 1.

In some embodiments R² is:

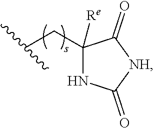

wherein s is selected from the group consisting of 0, 1, 2, and 3, and R^{e} is selected from hydrogen and C₁-C₆ alkyl.

In some embodiments, s is 0 and R^{e} is methyl.

In some embodiments, R² is:

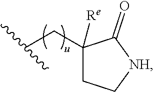

wherein u is selected from the group consisting of 0, 1, 2, and 3, and R^{e} is selected from hydrogen and C₁-C₆ alkyl.

In some embodiments, R² is:

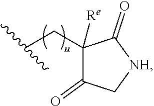

wherein u is selected from the group consisting of 0, 1, 2, and 3, and R^{e} is selected from hydrogen and C₁-C₆ alkyl.

In some embodiments, R² is:

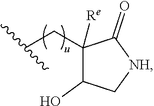

wherein u is selected from the group consisting of 0, 1, 2, and 3, and R^{e} is selected from hydrogen and C₁-C₆ alkyl.

In some embodiments, u is 0 and R^{e} is methyl.

In some embodiments, the compounds are compounds of formula (Id):

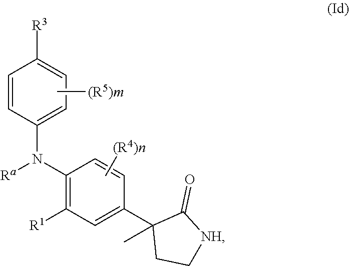

or pharmaceutically acceptable salts thereof, wherein R³ is —C₁-C₆ alkyl or —C₁-C₆ haloalkyl; and
the rest of the variables are as defined in formula (Ia).

In some embodiments, the compounds are compounds of formula (Ie):

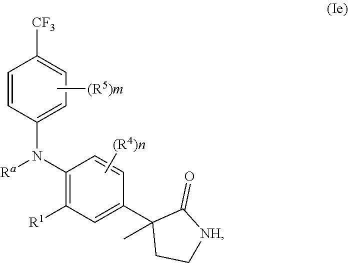

or pharmaceutically acceptable salts thereof, wherein the variables are as defined in formula (Id).

In some embodiments, R³ is C₁-C₆ alkyl or —C₁-C₆ haloalkyl.

In some embodiments, R³ is halogen.

In some embodiments, R³ is trifluoromethyl.

In some embodiments, m and n are both 0.

In some embodiments, R^{a} is hydrogen.

In some embodiments, the compound is a compound of Table 1, or a pharmaceutically acceptable salt thereof.

TABLE 1
List of compounds.

| Compound No. | Structure |
|---|---|
| 1 | 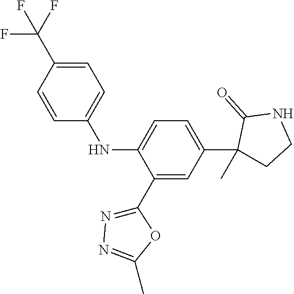 [SMILES: Cc1nnc(-c2cc(C3(C)CCNC3=O)ccc2Nc2ccc(C(F)(F)F)cc2)o1] |
| 2 | 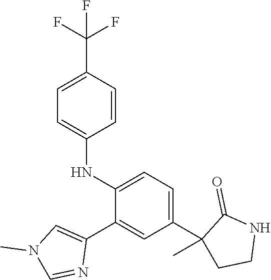 [SMILES: Cn1cnc(-c2cc(C3(C)CCNC3=O)ccc2Nc2ccc(C(F)(F)F)cc2)c1] |
| 3 | 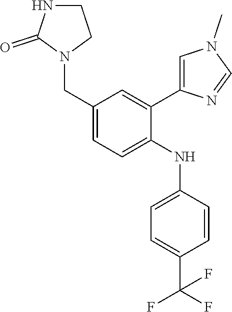 [SMILES: Cn1cnc(-c2cc(CN3CCNC3=O)ccc2Nc2ccc(C(F)(F)F)cc2)c1] |
| 4 | 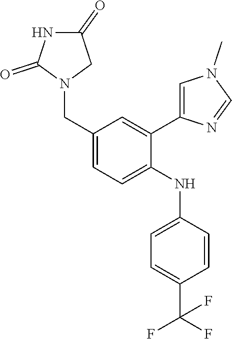 [SMILES: Cn1cnc(-c2cc(CN3CC(=O)NC3=O)ccc2Nc2ccc(C(F)(F)F)cc2)c1] |
| 5 | 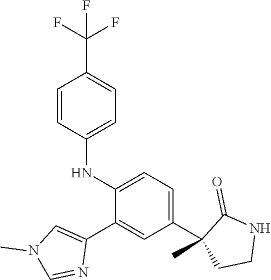 [SMILES: Cn1cnc(-c2cc([C@@]3(C)CCNC3=O)ccc2Nc2ccc(C(F)(F)F)cc2)c1] |
| | OR |
| | 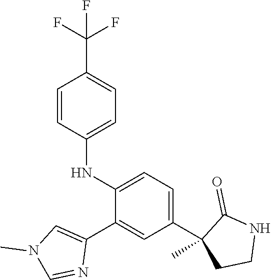 [SMILES: Cn1cnc(-c2cc([C@]3(C)CCNC3=O)ccc2Nc2ccc(C(F)(F)F)cc2)c1] |
| 6 | 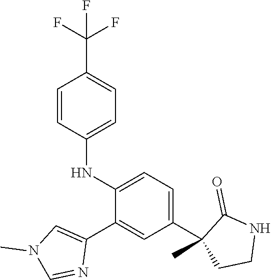 [SMILES: Cn1cnc(-c2cc([C@@]3(C)CCNC3=O)ccc2Nc2ccc(C(F)(F)F)cc2)c1] |
| | OR |
| | 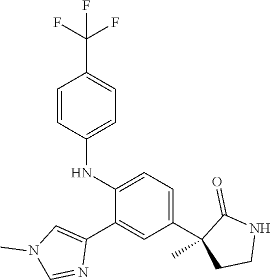 [SMILES: Cn1cnc(-c2cc([C@]3(C)CCNC3=O)ccc2Nc2ccc(C(F)(F)F)cc2)c1] |
| 7 | 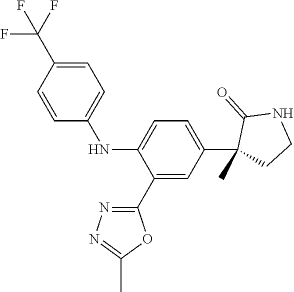 [SMILES: Cc1nnc(-c2cc([C@@]3(C)CCNC3=O)ccc2Nc2ccc(C(F)(F)F)cc2)o1] |
| | OR |
| | 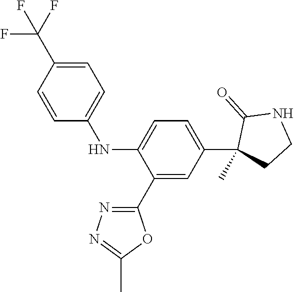 [SMILES: Cc1nnc(-c2cc([C@]3(C)CCNC3=O)ccc2Nc2ccc(C(F)(F)F)cc2)o1] |
| 8 | 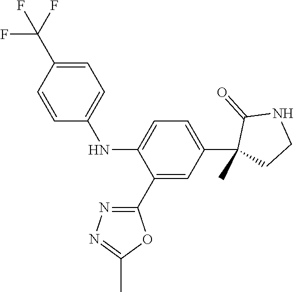 [SMILES: Cc1nnc(-c2cc([C@@]3(C)CCNC3=O)ccc2Nc2ccc(C(F)(F)F)cc2)o1] |
| | OR |
| | 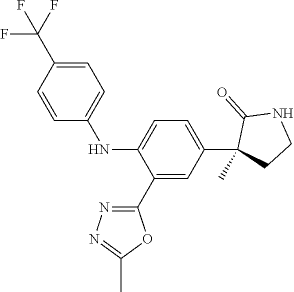 [SMILES: Cc1nnc(-c2cc([C@]3(C)CCNC3=O)ccc2Nc2ccc(C(F)(F)F)cc2)o1] |
| 9 | 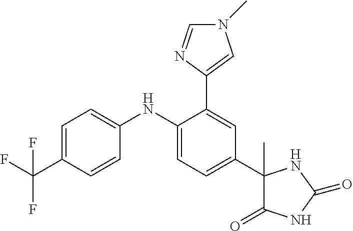 [SMILES: Cn1cnc(-c2cc(C3(C)NC(=O)NC3=O)ccc2Nc2ccc(C(F)(F)F)cc2)c1] |
| 10 | 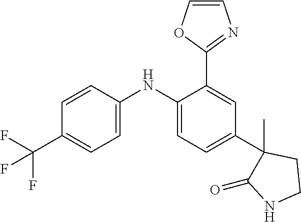 [SMILES: CC1(c2ccc(Nc3ccc(C(F)(F)F)cc3)c(-c3ncco3)c2)CCNC1=O] |
| 11 | 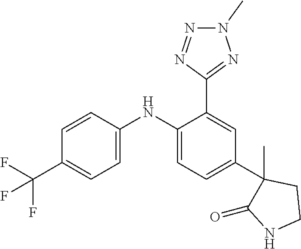 [SMILES: Cn1nnc(-c2cc(C3(C)CCNC3=O)ccc2Nc2ccc(C(F)(F)F)cc2)n1] |
| 12 | 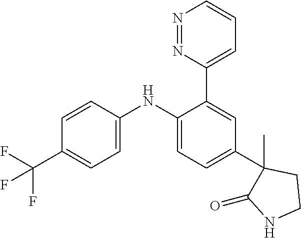 [SMILES: CC1(c2ccc(Nc3ccc(C(F)(F)F)cc3)c(-c3cccnn3)c2)CCNC1=O] |
| 13 | 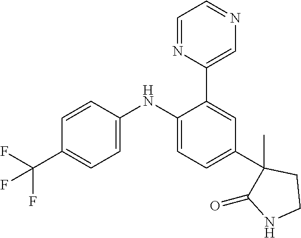 [SMILES: CC1(c2ccc(Nc3ccc(C(F)(F)F)cc3)c(-c3cnccn3)c2)CCNC1=O] |
| 14 | 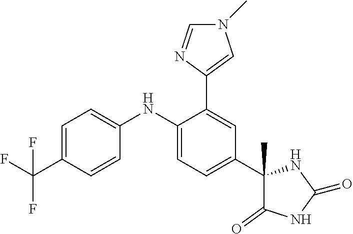 [SMILES: Cn1cnc(-c2cc([C@]3(C)NC(=O)NC3=O)ccc2Nc2ccc(C(F)(F)F)cc2)c1] |
| | OR |
| | 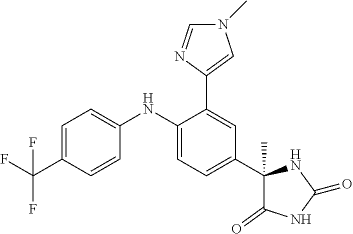 [SMILES: Cn1cnc(-c2cc([C@@]3(C)NC(=O)NC3=O)ccc2Nc2ccc(C(F)(F)F)cc2)c1] |
| 15 | 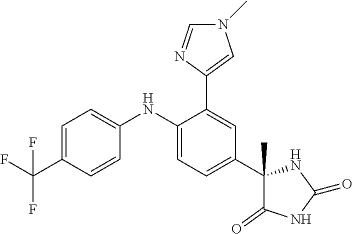 [SMILES: Cn1cnc(-c2cc([C@]3(C)NC(=O)NC3=O)ccc2Nc2ccc(C(F)(F)F)cc2)c1] |
| | OR |
| | 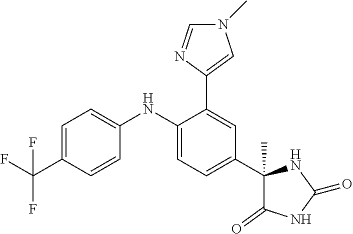 [SMILES: Cn1cnc(-c2cc([C@@]3(C)NC(=O)NC3=O)ccc2Nc2ccc(C(F)(F)F)cc2)c1] |
| 16 | 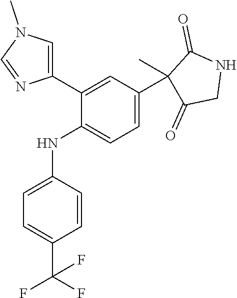 [SMILES: Cn1cnc(-c2cc(C3(C)C(=O)CNC3=O)ccc2Nc2ccc(C(F)(F)F)cc2)c1] |
| 17 | 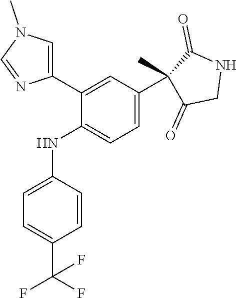 [SMILES: Cn1cnc(-c2cc([C@@]3(C)C(=O)CNC3=O)ccc2Nc2ccc(C(F)(F)F)cc2)c1] |
| 18 | 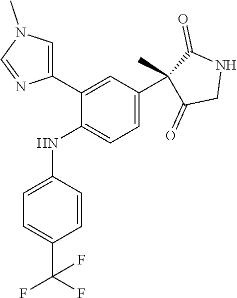 [SMILES: Cn1cnc(-c2cc([C@@]3(C)C(=O)CNC3=O)ccc2Nc2ccc(C(F)(F)F)cc2)c1] |
| | OR |
| | 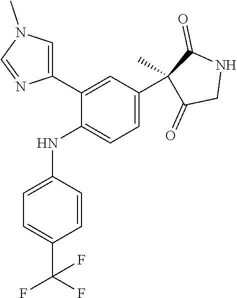 [SMILES: Cn1cnc(-c2cc([C@]3(C)C(=O)CNC3=O)ccc2Nc2ccc(C(F)(F)F)cc2)c1] |
| 19 | 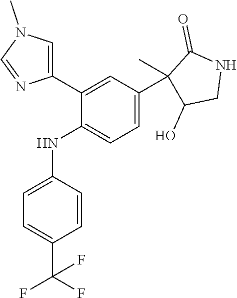 [SMILES: Cn1cnc(-c2cc(C3(C)C(=O)NCC3O)ccc2Nc2ccc(C(F)(F)F)cc2)c1] |
| 20 | 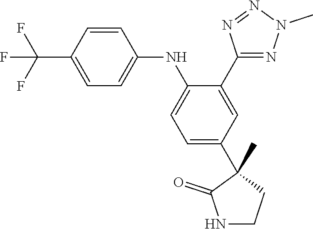 [SMILES: Cn1nnc(-c2cc([C@]3(C)CCNC3=O)ccc2Nc2ccc(C(F)(F)F)cc2)n1] |
| | OR |
| | 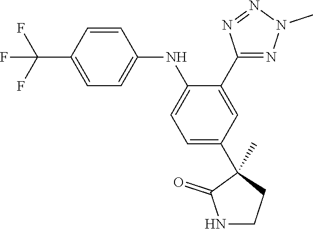 [SMILES: Cn1nnc(-c2cc([C@@]3(C)CCNC3=O)ccc2Nc2ccc(C(F)(F)F)cc2)n1] |
| 21 | 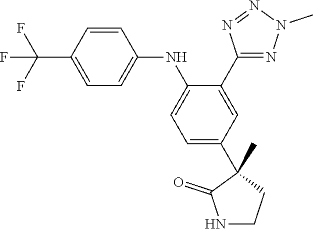 [SMILES: Cn1nnc(-c2cc([C@]3(C)CCNC3=O)ccc2Nc2ccc(C(F)(F)F)cc2)n1] |
| | OR |
| | 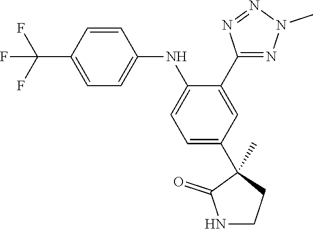 [SMILES: Cn1nnc(-c2cc([C@@]3(C)CCNC3=O)ccc2Nc2ccc(C(F)(F)F)cc2)n1] |
| 22 | 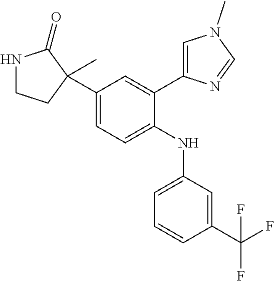 [SMILES: Cn1cnc(-c2cc(C3(C)CCNC3=O)ccc2Nc2cccc(C(F)(F)F)c2)c1] |

Also disclosed herein, in certain embodiments, is a compound of Table 1a, or a pharmaceutically acceptable salt thereof.

TABLE 1a
List of compounds.

| Compound No. | Structure | Compound No. | Structure |
|---|---|---|---|
| 23 | 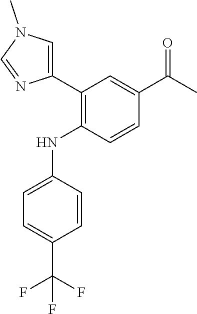 [SMILES: CC(=O)c1ccc(Nc2ccc(C(F)(F)F)cc2)c(-c2cn(C)cn2)c1] | 24 | 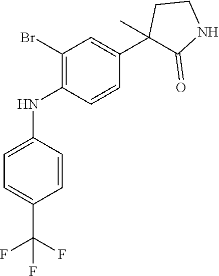 [SMILES: CC1(c2ccc(Nc3ccc(C(F)(F)F)cc3)c(Br)c2)CCNC1=O] |

### Pharmaceutical Compositions and Routes of Administration

Disclosed herein, in certain embodiments, are pharmaceutical compositions comprising: (a) a compound disclosed herein (e.g., a compound of formula (I), formula (I′), formula (I-1), formula (I-1′), formula (Ia), formula (Ia′), formula (Ib), formula (Ib′) formula (Ic), formula (Ic′), formula (Id), or formula (Je)) or a pharmaceutically acceptable salt thereof, and (b) a pharmaceutically acceptable excipient. In some embodiments, the excipient is selected from: inert solid diluents and fillers, diluents, including sterile aqueous solution and various organic solvents, permeation enhancers, solubilizers and adjuvants. In some embodiment, the pharmaceutical compositions are administered alone or in combination with other therapeutic agents. Such compositions are prepared in any suitable manner.

In some embodiments, the pharmaceutical compositions are administered in either single or multiple doses by any of the accepted modes of administration of agents having similar utilities, including rectal, buccal, intransal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. In some embodiments, the forms in which the novel compositions of the present disclosure are incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also used for injection. In some embodiments, ethanol, glycerol, propylene glycol, liquid polyethylene glycol, and the like (and suitable mixtures thereof), cyclodextrin derivatives, and vegetable oils are employed. In some embodiments, the proper fluidity is maintained, for example, by the use of a coating, such as lecithin, by the maintenance of the required particle size in the case of dispersion and by the use of surfactants. In some embodiments, the prevention of the action of microorganisms is brought about by various antibacterial and antifungal agents, for example, parabens, chlorobutanol, phenol, sorbic acid, thimerosal, and the like.

Sterile injectable solutions are prepared by incorporating a compound according to the present disclosure in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the compounds disclosed herein. In some embodiments, administration is via capsule or enteric coated tablets, or the like. In some embodiments, in making the pharmaceutical compositions that include at least one compound described herein, the active ingredient is usually diluted by an excipient and/or enclosed within such a carrier that is in the form of a capsule, sachet, paper or other container. In some embodiments, when the excipient serves as a diluent, it is in the form of a solid, semi-solid, or liquid material (as above), which acts as a vehicle, carrier or medium for the active ingredient. Thus, in some embodiments, the compositions are e in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. In some embodiments, the formulations additionally include: lubricating agents such as talc, magnesium stearate, and mineral oil; wetting agents; emulsifying and suspending agents; preserving agents such as methyl and propylhydroxy-benzoates; sweetening agents; and flavoring agents.

In some embodiments, the compositions of the disclosure are formulated so as to provide quick, sustained or delayed release of the active ingredient after administration to the patient. Controlled release drug delivery systems for oral administration include osmotic pump systems and dissolutional systems containing polymer-coated reservoirs or drug-polymer matrix formulations. Another formulation for use in the methods of the present disclosure employs transdermal delivery devices (“patches”). In some embodiments, such transdermal patches are used to provide continuous or discontinuous infusion of the compounds of the present disclosure in controlled amounts. The construction and use of transdermal patches for the delivery of pharmaceutical agents is well known in the art. In some embodiments, such patches are constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. In some embodiments, when referring to these preformulation compositions as homogeneous, it is meant that the active ingredient is dispersed evenly throughout the composition so that the composition is readily subdivided into equally effective unit dosage forms such as tablets, pills and capsules.

In some embodiments, the tablets or pills of the present disclosure are coated or otherwise compounded to provide a dosage form affording the advantage of prolonged action, or to protect from the acid conditions of the stomach. For example, in some embodiments, the tablet or pill comprises an inner dosage and an outer dosage component, the latter being in the form of an envelope over the former. In some embodiments, the two components are separated by an enteric layer that serves to resist disintegration in the stomach and permit the inner component to pass intact into the duodenum or to be delayed in release. In some embodiments, a variety of materials are used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. In some embodiments, the liquid or solid compositions contain suitable pharmaceutically acceptable excipients as described supra. In some embodiments, the compositions are administered by the oral or nasal respiratory route for local or systemic effect. In some embodiments, compositions in pharmaceutically acceptable solvents are nebulized by use of inert gases. In some embodiments, nebulized solutions are inhaled directly from the nebulizing device or the nebulizing device is attached to a facemask tent, or intermittent positive pressure breathing machine. In some embodiments, solution, suspension, or powder compositions are administered, orally or nasally, from devices that deliver the formulation in an appropriate manner.

### Methods of Treatment

Disclosed herein, in certain embodiments, are methods of treating a disease or disorder modulated by abnormal activity of a TEAD isoform (e.g., TEAD1 and/or TEAD4) (e.g., hyperactivation of TEAD transcriptional complex) in an individual in need thereof, comprising administering to the individual a compound of formula (I), formula (I′), formula (I-1), formula (I-1′), formula (Ia), formula (Ia′), formula (Ib), formula (Ib′), formula (Ic), formula (Ic′), formula (Id), or formula (Ie), or a pharmaceutically acceptable salt thereof (e.g., a therapeutically effective amount of a compound of formula (I), formula (I′), formula (I-1), formula (I-1′), formula (Ia), formula (Ia′), formula (Ib), formula (Ib′), formula (Ic), formula (Ic′), formula (Id), or formula (Ie), or a pharmaceutically acceptable salt thereof).

TEA domain transcription factors (TEADs) are downstream effectors of the Hippo signaling pathway. Currently, four isoforms of TEAD have been identified—TEAD1, TEAD2, TEAD3, and TEAD4. The TEAD isoforms share highly similar structures. The N-terminus of the four isoforms share a highly conserved 68-amino acid TEA/ATTS DNA-binding domain, which binds to the MCAT element (50-CATTCCA/T-30). The C-terminus contains the transactivation domain which recruits transcriptional coactivators YAP/TAZ.

The expression of TEAD proteins is up-regulated in many cancer types including gastric, colorectal, breast, and prostate cancers. TEAD hyperactivation plays a role in tumor progression, metastasis, cancer metabolism, immunity, and drug resistance and is correlated with poor survival in patients.

Selective binding of TEAD1 and/or TEAD4 is beneficial for optimizing anti-tumor efficacy while minimizing undesired effects. Inhibition of TEAD3 has been associated with off-target toxicity (e.g., kidney toxicity), while inhibition of TEAD2 may be pro-proliferative.

The TEAD isoform selectivity demonstrated by compounds disclosed herein is an advantage over TEAD inhibitors known in the art. For example, as demonstrated by Tang and Post using thermal shift assays, TEAD inhibitor VT3989 primarily interacts with TEAD1-3. The reported thermal shift data are provided in Table 2 below (Tang and Post, “The TEAD autopalmitoylation inhibitor VT3989 improves efficacy and increases durability of efficacy of osimertinib in preclinical EGFR mutant tumor models,” Proceedings of the American Association for Cancer Research Annual Meeting 2022; 2022 Apr. 8-13. Philadelphia (PA): AACR; Poster #5364). The data provided in Table 2 shows that VT3989 provides minimal selectivity over TEAD2 and 3, TEAD isoforms for which inhibition is undesirable. Furthermore, VT3989 shows lower levels of binding to TEAD4, a TEAD isoform for which inhibition is desirable. Each change in ΔTm (° C.) of 2.5° C. may be associated with an approximately 10-fold difference in binding affinity (Bhayani et al.; “Determination of dissociation constants of protein ligands by thermal shift assay,” Biochemical and Biophysical Research Communications, 2022, 560:1-6.)

TABLE 2
Thermal shift assay of TEAD isoforms 1-4 treated with TEAD
inhibitor VT3989 compared to inactive analog control.

| | | Inactive |
|---|---|---|
| Isoform | VT3989 | Analog |
| TEAD1 ΔTm (° C.) | 11.6 | 0.2 |
| TEAD2 ΔTm (° C.) | 8.8 | 0.5 |
| TEAD3 ΔTm (° C.) | 8.5 | 0.2 |
| TEAD4 ΔTm (° C.) | 4.6 | 0.2 |

In some embodiments, the compounds disclosed herein selectively bind one or more TEAD isoforms. In certain embodiments, the compounds disclosed herein selectively bind TEAD1. In certain embodiments, the compounds disclosed herein selectively bind TEAD4. In certain embodiments, the compounds disclosed herein selectively bind TEAD1 and TEAD4. In some embodiments, the binding selectivity for TEAD1 is 10-fold, 100-fold, 1,000-fold, or 10,000-fold over TEAD2 and/or TEAD3. In some embodiments, the binding selectivity for TEAD4 is 10-fold, 100-fold, 1,000-fold, or 10,000-fold over TEAD2 and/or TEAD3. Evidence of the TEAD isoform selectivity demonstrated by compounds disclosed herein is provided in Example 26 below.

In some embodiments, the compounds disclosed herein are useful as a medical therapy for treating a disease or condition mediated by hyperactivation of YAP/TAZ-TEAD (e.g., TEAD1 and/or TEAD4) transcriptional co-activator complex. In some embodiments, the compounds disclosed herein are useful as a medical therapy for treating a disease or condition characterized by the hyperactivation of TEAD (e.g., TEAD1, TEAD2, TEAD3, and TEAD4 isoforms). In some embodiments, the disease or condition is characterized by the overexpression or genomic fusion or amplification of TEAD. TEAD1 and TEAD4 experience recurrent onco-fusions; for example, TEAD1-PARVA. These fusions drive higher expression of TEADs and may result in enhanced transcription of TEAD-target genes. Amplifications (genomic copy number gains) of TEAD4 occur in diverse cancers as part of the 12p13 locus in, e.g., ovarian and uterine carcinoma as well as testicular germ cell tumors. These genomic amplifications of TEAD4 are associated with strong increase in its mRNA expression and may drive high TEAD-YAP/TAZ transcriptional activity.

In certain embodiments, the TEAD isoform is TEAD1. In certain embodiments, the TEAD isoform is TEAD4. In some embodiments, the disease, disorder or condition is a cancer characterized by abnormal TEAD transcriptional complex activity (e.g., hyperactivation). Such cancers include but are not limited to breast cancer, lung cancer, gastric cancer, colorectal cancer, pancreatic cancer including pancreatic adenocarcinoma, mesothelioma including malignant mesothelioma, hepatocellular cancer, prostate cancer, head and neck cancer, renal cell carcinoma, and medulloblastomas. In some embodiments, the cancer is selected from pancreatic adenocarcinoma, hepatocellular cancer, breast cancer, and malignant mesothelioma. In certain embodiments, the pancreatic cancer is pancreatic adenocarcinoma. In certain embodiments, the cancer is malignant mesothelioma.

Also provided herein is a compound or pharmaceutically acceptable salt disclosed herein, or a pharmaceutical composition disclosed herein, for use in treating a disease or disorder mediated by hyperactivation of a TEAD isoform selected from TEAD1 and TEAD4 in a subject in need thereof.

In another aspect, provided herein is use of a compound or pharmaceutically acceptable salt disclosed herein, or a pharmaceutical composition disclosed herein in the manufacture of a medicament for treating a disease or disorder mediated by hyperactivation of a TEAD isoform selected from TEAD1 and TEAD4 in a subject in need thereof.

In some embodiments, the cancer is a breast cancer. In some embodiments, the cancer is a lung cancer. In some embodiments, the cancer is a gastric cancer. In some embodiments, the cancer is a colorectal cancer. In some embodiments, the cancer is a prostate cancer. In some embodiments, the cancer is a head and neck cancer. In some embodiments, the cancer is a renal cell carcinoma. In some embodiments, the cancer is a medulloblastoma. In some embodiments, the cancer is a pancreatic cancer. In some embodiments, the cancer is a hepatocellular cancer. In some embodiments, the cancer is a metastatic cancer. In some embodiments, the cancer is a metastatic breast cancer. In some embodiments, the cancer is a metastatic lung cancer. In some embodiments, the cancer is a metastatic gastric cancer. In some embodiments, the cancer is a metastatic colorectal cancer. In some embodiments, the cancer is a metastatic prostate cancer. In some embodiments, the cancer is a metastatic head and neck cancer. In some embodiments, the cancer is a metastatic renal cell carcinoma. In some embodiments, the cancer is a metastatic mesothelioma. In some embodiments, the cancer is a metastatic pancreatic cancer. In some embodiments, the cancer is a metastatic hepatocellular cancer.

In some embodiments, the disclosure provides for compounds of formula (I), formula (I′), formula (I-1), formula (I-1′), formula (Ia), formula (Ia′), formula (Ib), formula (Ib′), formula (Ic), formula (Ic′), formula (Id), or formula (Ie) for modulating TEAD activity. In some embodiments, the disclosure provides for pharmaceutically acceptable salts of compounds of formula (I), formula (I′), formula (I-1), formula (I-1′), formula (Ia), formula (Ia′), formula (Ib), formula (Ib′), formula (Ic), formula (Ic′), formula (Id), or formula (Ie) for modulating TEAD activity.

In some embodiments, the disclosure provides for compounds of formula (I), formula (I′), formula (I-1), formula (I-1′), formula (Ia), formula (Ia′), formula (Ib), formula (Ib′), formula (Ic), formula (Ic′), formula (Id), or formula (Ie) or pharmaceutically acceptable salts thereof for use in medical therapy.

In some embodiments, the compounds and pharmaceutically acceptable salts thereof or pharmaceutical compositions disclosed herein are administered alone or in combination with one or more other therapeutic agents. In some embodiments, the other therapeutic agent is any suitable chemotherapeutic agent. In some embodiments, the one or more other therapeutic agents are selected from modulators of other transcription factors. In some embodiments, the modulators of other transcription factors are selected from modulators of YAP, modulators of EGFR, or modulators of MEK.

In some embodiments, the one or more other therapeutic agents are administered concurrently with the compounds or pharmaceutically salts thereof or pharmaceutical compositions disclosed herein. In some embodiments, the one or more other therapeutic agents are administered sequentially with the compounds or pharmaceutically salts thereof or pharmaceutical compositions disclosed herein. In some embodiments, the one or more other therapeutic agents are administered before the compounds or pharmaceutically salts thereof or pharmaceutical compositions disclosed herein. In some embodiments, the one or more other therapeutic agents are administered after the compounds or pharmaceutically salts thereof or pharmaceutical compositions described disclosed.

### EXAMPLES

The present disclosure will be further illustrated in the following Examples which are given for illustration purposes only and are not intended to limit the disclosure in any way.

### Abbreviations

ACN acetonitrile
B₂Pin₂ bis(pinacolato)diboron
Boc tert-butyloxycarbonyl
CAN ceric ammonium nitrate
DAST diethylaminosulfur trifluoride
DBU 1,8-diazabicyclo (5.4.0) undec-7-ene
DCC dicyclohexylcarbodiimide
DCE 1,1-dichloroethane
DCM dichloromethane
DEA diethanolamine
DEAD diethyl azodicarboxylate
DIAD diisopropyl azodicarboxylate
DIBAL Diisobutylaluminium hydride
DIPEA N, N-diisopropylethylamine, Hünig's base
DMA N, N-dimethylacetamide
DMAP 4-(dimethylamino) pyridine
DMF N, N-dimethylformamide
DMSO dimethylsulfoxide
EDC 1-Ethyl-3-(3-dimethylaminopropyl) carbodiimide
EtOAc/EA ethyl acetate
eq equivalent(s)
h hours
HATU N-[(dimethylamino)-1H-1,2,3-triazolo-[4,5-b]pyridin-1-ylmethylene]-N-methylmethanaminium hexafluorophosphate N-oxide
HBTU (1H-benzotriazol-1-yloxy) (dimethylamino)-N, N-dimethylmethaniminium hexafluorophosphate
hex hexane
HOBT N-hydroxybenzotriazole
HPLC high Pressure Liquid Chromatography
LAH lithium aluminium hydride
IPA isopropyl alcohol
LCMS Liquid Chromatography-Mass Spectrometry
LDA Lithium diisopropylamide
LiHMDS Lithium bis(trimethylsilyl)amide
mCPBA meta-chloroperoxybenzoic acid
MI Molecular Ion
Min minutes
MgSO₄ anhydrous magnesium sulfate
MS mass spectrometry
MW microwave
NBS/NBRS N-bromosuccinamide
NCS N-chlorosuccinamide
NFOBS/NFOBRS N-fluoro-o-benzenedisulfonimide
NFSI N-fluorobenzenesulfonimide
NHS N-hydroxysuccinimide
NIS N-iodosuccinamide
NMM N-methylmorpholine
NMP 1-methyl-2-pyrrolidinone
NMR Nuclear Magnetic Resonance
PdCl₂(PPh₃)₂ Bis(triphenylphosphine)palladium chloride
Pd(dppf)₂Cl₂ [1,1′-bis(diphenylphosphino)ferrocene]dichloropalladium(II)
Pd(dppf)₂Cl₂·DCM [1,1′-bis(diphenylphosphino)ferrocene]dichloropalladium(II) complex with DCM
(Pd(dba)₂) bis(dibenzylideneacetone)palladium
(Pd₂(dba)₃) tris(dibenzylideneacetone)palladium
Rbf/RB round bottomed flask
RM reaction mixture
RP reverse phase
RT Retention Time
SCX-2 a silica-based sorbent with a chemically bonded propylsulfonic acid functional group
SFC supercritical fluid chromatography
TBAF tetra-n-butylammonium fluoride
TBDMS tert-butyldimethylsilyl
TEA triethylamine
TFAA trifluoroacetic anhydride
TFA trifluoroacetic acid
THE tetrahydrofuran
TLC thin layer chromatography
TMSCN trimethylsilyl cyanide
TPP tripotassium phosphate
Ts/Tos toluenesulfonyl
Xantphos (9,9-dimethyl-9H-xanthene-4,5-diyl)bis(diphenylphosphane)
XPhos-Pd-G1 2-Dicyclohexylphosphino-2′,4′,6′-triisopropyl-1,1′-biphenyl) [2-(2-aminoethyl) phenyl)]palladium(II) chloride
XPhos-Pd-G2 Chloro(2-dicyclohexylphosphino-2′,4′,6′-triisopropyl-1,1′-biphenyl) [2-(2′-amino-1,1′-biphenyl)]palladium(II)

### Analytical Methods

Commercially available starting materials, reagents and dry solvents were used as supplied. Flash column chromatography or glass column chromatography was performed using Merck silica gel 230-400 mesh size. Flash chromatography was also performed on combi-flash RF Teledyne Isco machine. Preparative TLC was performed on Merck plates.

### Liquid Chromatography-Mass Spectrometry Methods

### Method-A

Method Name: —UC02_FAR1, Machine Details: —Water Acquity UPLC-H Class equipped with PDA and Acquity SQ detector, Column: Waters X-bridge C18, 50*2.1 mm, 2.5 micron, Column temperature: 35° C., Auto sampler temperature: 15° C., Mobile Phase A: 0.1% Formic acid in Milli Q water (PH=2.70), Mobile Phase B: 0.1% Formic acid in Milli Q water: Acetonitrile (10:90); Mobile phase gradient details:: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Flow rate: −0.8 mL/min, Run Time: −4 min, UV Detection Method: −PDA, Wavelength: −200-500 nm; Mass parameter: Probe: −ESI, Mode of Ionisation: —positive and negative, Cone voltage: −30V and 10 V, capillary voltage: −3.0 KV, Extractor Voltage: −1 V, R^{f} Lens: −0.1 V, Temperature of source: −120° C., Temperature of Desolvation: −400° C. Cone Gas Flow: −100 L/hour, Desolvation Gas flow: −800 L/hour.

### Method-B

Method Name: −UC03_ABR2, Machine Details: —Waters Acquity Ultraperfomance LC connected with PDA and equipped with SQ detector, Column: Waters X-bridge C18, 50*4.6 mm, 3.5 micron, Column temperature: 35° C., Auto sampler temperature: 15° C., Mobile Phase A: 5 mM Ammonium Bicarbonate (pH=8.00) in Milli Q water, Mobile Phase B: Acetonitrile; Mobile phase gradient details: T=0 min (97% A, 3% B) Flow rate=1.0 ml/min, T=0.20 min (97% A, 3% B) Flow rate=1.0 ml/min; gradient to T=2.70 min (20% A, 80% B) Flow rate=1.0 ml/min; gradient to T=3.0 min (0% A, 100% B) Flow rate=1.2 ml/min; T=3.50 min (0% A, 100% B) Flow rate=1.2 ml/min; T=3.51 min (97% A, 3% B) Flow rate=1.0 ml/min; end of run at T=4.0 min (97% A, 3% B) Flow rate=1.0 ml/min, Run Time: −4 min, UV Detection Method: −PDA, Wavelength: −195 nm-500 nm; Mass parameter: Probe: −ESI, Mode of Ionisation: —Positive and Negative, Cone voltage: −30 and 10 V, capillary voltage: −3.0 KV, Extractor Voltage: −2 V, Rf Lens: −0.1 V, Temperature of source: −120° C., Temperature of Probe: −400° C., Cone Gas Flow: −100 L/Hr, Desolvation Gas flow: −800 L/Hr.

### High-Performance Liquid Chromatography Methods

### Method-A

Method Name: —HP04_BR1 Machine Details: —Water alliance e2695 with 2998 PDA detector, Column temperature: 25° C., Auto sampler temperature: 25° C., Mobile Phase A: 0.1% ammonium hydroxide solution in HPLC water Mobile Phase B: 100% ACETONITRILE; Mobile phase gradient details: T=0 min (10% A, 90% B) flow: 1 mL/min; T=7 min (90% A, 10% B) flow: 1 mL/min; gradient to T=9 min (100% A, 0% B) flow: 1 mL/min; gradient to T=14 min (100% A, 0% B) flow: 1 mL/min; T=14.01 min (10% A, 90% B) flow: 1 mL/min; gradient to T=17 min (10% A, 90% B) flow: 1 mL/min; end of run at T=17 min (10% A, 90% B), Flow rate: 1 mL/min, Run Time: −17 min, UV Detection Method: −PDA.

### Method-B

Method Name: —HP05_TFAR1.Machine Details: AGILENT TECHNOLOGY 1260 infinity series with PDA detector, Column temperature: 25° C., Auto sampler temperature: 25° C., Mobile Phase A: 0.05% Trifluoroacetic acid in HPLC water Mobile Phase B: 100% Acetonitrile; Mobile phase gradient details T=0 min (90% A, 10% B) flow: 1 mL/min; T=7 min (10% A, 90% B) flow: 1 mL/min; gradient to T=9 min (0% A, 100% B) flow: 1 mL/min; gradient to T=14 min (0% A, 100% B) flow: 1 mL/min; T=14.01 min (90% A, 10% B) flow: 1 mL/min; gradient to T=17 min (90% A, 10% B) flow: 1 mL/min; end of run at T=17 min (90% A, 10% B), Flow rate: 1 mL/min, Run Time: −17 min, UV Detection Method: −PDA.

### Method-C

Method Name: —HP06_TFAR1 Machine Details: —AGILENT TECHNOLOGY 1100series with PDA detector, Column temperature: 25° C., Auto sampler temperature: 25° C., Mobile Phase A: 0.05% Trifluoroacetic acid in HPLC water Mobile Phase B: 100% Acetonitrile; Mobile phase gradient details T=0 min (90% A, 10% B) flow: 1 mL/min; T=7 min (10% A, 90% B) flow: 1 mL/min; gradient to T=9 min (0% A, 100% B) flow: 1 mL/min; gradient to T=14 min (0% A, 100% B) flow: 1 mL/min; T=14.01 min (90% A, 10% B) flow: 1 mL/min; gradient to T=17 min (90% A, 10% B) flow: 1 mL/min; end of run at T=17 min (90% A, 10% B), Flow rate: 1 mL/min, Run Time: −17 min, UV Detection Method: —PDA.

### NMR

¹H Nuclear magnetic resonance (NMR) spectroscopy was carried out using a Bruker Avance-400 instrument operating at 400 MHz using the stated solvent at room temperature unless otherwise stated. Samples were prepared as solutions in a suitable deuterated solvent and referenced to the appropriate internal non-deuterated solvent peak or tetramethylsilane. Chemical shifts were recorded in ppm (δ) downfield of tetramethylsilane. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; m, multiplet; br, broad; br s=broad singlet.

### Purification Methods

### Preparative Purification by Reverse Phase HPLC

### Preparatory HPLC Method-A

Biotage-FC-01 with binary pump with UV/Visible wave-length detector, Column: YMC, 120 g, 50 μm, column temperature: room temp., mobile phase A: 0.1% formic acid in water, mobile phase B: Acetonitrile: mobile phase gradient details: t=0.01 min (100% A, 0% B); t=3 min (85% A, 15% B); gradient to t=25 min (55% A, 45% B); t=35 min (0% A, 100% B); gradient to end of run at t=45 min (100% A, 0% B); flow rate: 80 mL/min, analysis time 45 min.

### Synthesis

Several methods for the chemical synthesis of compounds of the present application are described herein. These and/or other well-known methods may be modified and/or adapted in various ways to facilitate the synthesis of additional compounds within the scope of the present application and claims. Such alternative Methods and modifications should be understood as being within the spirit and scope of this application and claims. Accordingly, Methods set forth in the following descriptions, Schemes and Examples are intended for illustrative purposes and are not to be construed as limiting the scope of the disclosure.

### Example 1—Synthesis of 3-methyl-3-(3-(5-methyl-1,3,4-oxadiazol-2-yl)-4-((4-(trifluoromethyl) phenyl) amino) phenyl) pyrrolidin-2-one (Compound 1)

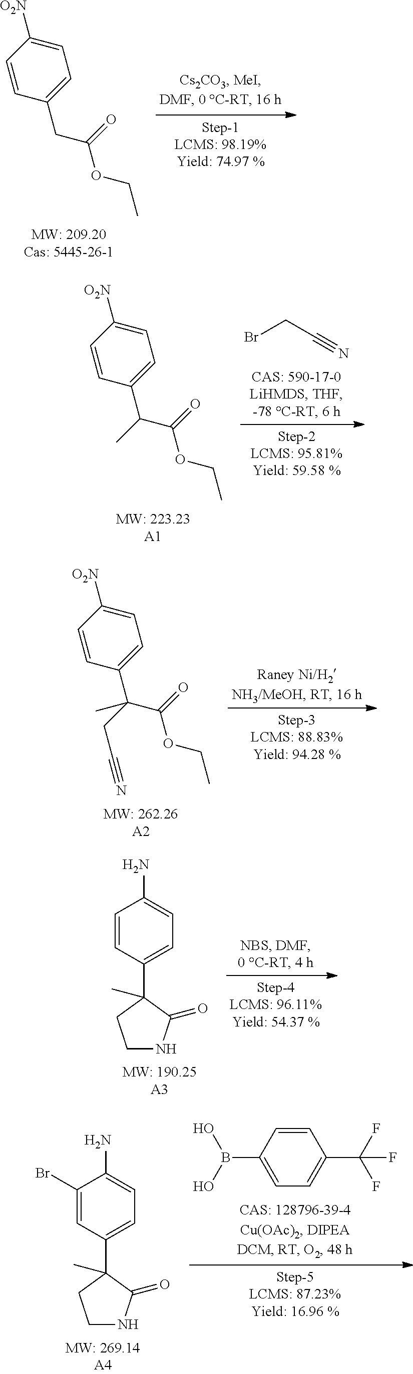

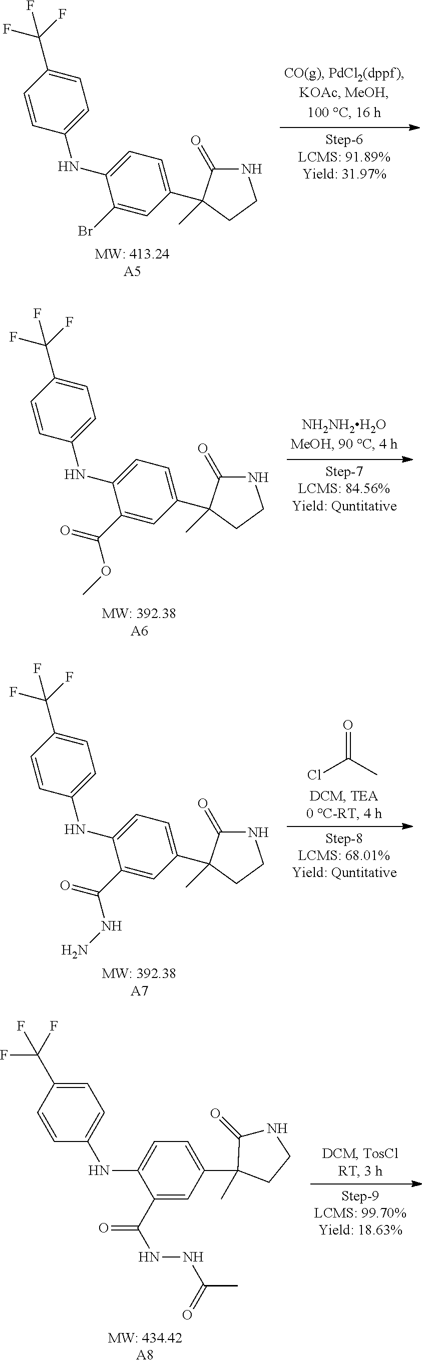

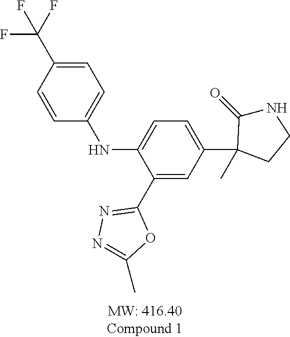 [SMILES: Cc1nnc(-c2cc(C3(C)CCNC3=O)ccc2Nc2ccc(C(F)(F)F)cc2)o1]

### Step-1:

A solution of CAS:5445-26-1 (0.50 g, 2.390 mmol, 1.0 eq) and Cs₂CO₃ (1.16 g, 3.585 mmol, 1.5 eq) in DMF (5 mL) was stirred at RT for 15 min and cooled to 0° C. Methyl iodide (0.37 g, 2.629 mmol, 1.1 eq) was added to this solution at 0° C. The resulting mixture was stirred at RT for 16 h. The reaction was monitored on TLC (using EtOAc:Hexane; 0.5:9.5 as mobile phase) which confirmed that the reaction was complete after 16 h of stirring at RT. The resulting reaction mixture was diluted with ice water (50 mL) and extracted with EtOAc (3×20 mL). The combined organic layers were dried over Na₂SO₄ and concentrated under reduced pressure to afford 0.720 g crude. The obtained crude material was purified by flash column chromatography using silica (230-400 mesh) as stationary phase (0-9% EtOAc in Hexane) yielding ethyl 2-(4-nitrophenyl) propanoate (A1) (0.400 g, 1.791 mmol, Yield: 74.97%).

¹H NMR (DMSO-d₆, 400 MHz): δ ppm, 8.21 (dd, J=7.2, 2.0 Hz, 2H), 7.58 (dd, J=7.2, 2.0 Hz, 2H), 4.11-4.00 (m, 3H), 1.43 (d, J=7.2 Hz, 3H), 1.13 (t, J=7.2 Hz, 3H).

LCMS (Method B): 2.90 min, 98.19%, 254.0 nm, MS: ES− 222.19 (M−1)

### Step-2:

To a stirred solution of ethyl 2-(4-nitrophenyl) propanoate (A1) (0.20 g, 0.896 mmol, 1.0 eq) in THE (2 mL) was added LiHMDS (2.0 m in THF) (0.94 mL, 1.881 mmol, 2.1 eq) at −78° C. The mixture was stirred at −78° C. for 1 h. Then CAS: 590-17-0 (0.12 g, 1.075 mmol, 1.2 eq) was added at −78° C. The resulting mixture was stirred at RT for 6 h. The reaction was monitored on TLC (using EtOAc:Hexane; 2.0:8.0 as mobile phase) which confirmed that the reaction was complete after 6 h of stirring at RT. The resulting reaction mixture was diluted with water (30 mL) and extracted with EtOAc (3×20 mL). The combined organic layers were dried over Na₂SO₄ and concentrated under reduced pressure to afford 0.32 g crude. The obtained crude material was purified by flash column chromatography using silica (230-400 mesh) as stationary phase (0-2% EtOAc in Hexane) yielding ethyl 3-cyano-2-methyl-2-(4-nitrophenyl) propanoate (A2) (0.14 g, 0.533 mmol, Yield: 59.58%).

¹H NMR (CDCl₃, 400 MHz): δ ppm, 8.28 (dd, J=7.2, 2.0 Hz, 2H), 7.54 (dd, J=7.2, 2.0 Hz, 2H), 4.27-4.20 (m, 2H), 3.03 (q, J=16.8 Hz, 2H), 1.88 (s, 3H), 1.24 (t, J=7.2 Hz, 3H).

Note: Impurity peaks were observed in 1H NMR.

LCMS (Method B): 2.75 min, 95.81%,254.0 nm, MS: ES− 261.91 (M−1)

### Step-3:

To a stirred solution of ethyl 3-cyano-2-methyl-2-(4-nitrophenyl) propanoate (A2) (0.80 g, 3.051 mmol, 1.0 eq) in ammonia solution 7 N in methanol (8 mL) was added Raney Ni (0.80 g) at RT. The resulting mixture was stirred at RT under H₂(g) atmosphere (200 psi) for 16 h. The reaction was monitored via TLC (using 100% EtOAc as mobile phase) which confirmed that the reaction was complete after 16 h of stirring at RT. The resulting reaction mixture was directly filtered with celite. The filtrate was dried over Na₂SO₄ and concentrated under reduced pressure to afford 1.0 g crude. The obtained crude material was purified by flash column chromatography using silica (230-400 mesh) as stationary phase (0-4% MeOH in Dichloromethane) yielding 3-(4-aminophenyl)-3-methylpyrrolidin-2-one (A3) (0.6 g, 3.153 mmol, Yield: 94.28%).

¹H NMR (DMSO-d₆, 400 MHz): δ ppm, 7.64 (br s, 1H), 7.03 (dd, J=6.8, 2.0 Hz, 2H), 6.51 (dd, J=6.8, 2.0 Hz, 2H), 5.01 (br s, 2H), 3.19-3.14 (m, 1H), 3.08-3.02 (m, 1H), 2.32-2.27 (m, 1H), 2.07-1.99 (m, 1H), 1.29 (s, 3H).

LCMS (Method A): 0.192 min, 88.83%, 220.0 nm, MS: ES+ 191.05 (M+1)

### Step-4:

To a stirred solution of 3-(4-aminophenyl)-3-methylpyrrolidin-2-one (A3) (0.26 g, 1.368 mmol, 1.0 eq) in DMF (2 mL) was added N-bromosuccinimide (0.17 g, 0.957 mmol, 0.7 eq) at 0° C. The resulting mixture was stirred at RT for 4 h. The reaction was monitored on TLC (using 100% EtOAc as mobile phase) which confirmed that the reaction was complete after 4 h of stirring at RT. The resulting reaction mixture was diluted with water (10 mL) and extracted with EtOAc (3×20 mL). The combined organic layers were dried over Na₂SO₄ and concentrated under reduced pressure to afford 0.20 g crude yielding 3-(4-amino-3-bromophenyl)-3-methylpyrrolidin-2-one (A4) (0.20 g, 0.743 mmol, Yield: 54.37%).

¹H NMR (DMSO-d₆, 400 MHz): δ ppm, 7.73 (br s, 1H), 7.32 (s, 1H), 7.09 (d, J=8.4 Hz, 1H), 6.75 (d, J=8.4 Hz, 1H), 5.19 (s, 2H), 3.21-3.15 (m, 1H), 3.09-3.03 (m, 1H), 2.33-2.27 (m, 1H), 2.09-2.02 (m, 1H), 1.29 (s, 3H).

LCMS (Method A): 1.467 min, 96.11%, 254.0 nm, MS: ES+ 191.05 (M, M+2: 269.0,271.0)

### Step-5:

To a stirred solution of 3-(4-amino-3-bromophenyl)-3-methylpyrrolidin-2-one (A4) (0.50 g, 1.858 mmol, 1.0 eq), CAS: 128796-39-4 (0.53 g, 2.787 mmol, 1.5 eq) in DCM (10 mL) was added DIPEA (0.71 g, 5.574 mmol, 3.0 eq) at RT then Cu(OAc)₂ (0.50 g, 2.787 mmol, 1.5 eq) was added and the resulting mixture was stirred at RT for 48 h under O₂ atmosphere. The reaction was monitored via TLC (using 100% EtOAc as mobile phase) which confirmed that the reaction was complete after 48 h of stirring at RT. The resulting reaction mixture was diluted with water (20 mL) and extracted with DCM (3×20 mL). The combined organic layers were dried over Na₂SO₄ and concentrated under reduced pressure to afford 0.6 g crude. The obtained crude material was purified by RP column chromatography (0-85% ACN in Water) yielding 3-(3-bromo-4-((4-(trifluoromethyl) phenyl) amino) phenyl)-3-methylpyrrolidin-2-one (A5) (0.13 g, 0.314 mmol, Yield: 16.93%).

¹H NMR (DMSO-d₆, 400 MHz): δ ppm, 8.26 (s, 1H), 7.87 (s, 1H), 7.69 (d, J=2.0 Hz, 1H), 7.48 (d, J=8.8 Hz, 2H), 7.43-7.34 (m, 2H), 6.95 (d, J=8.4 Hz, 2H), 3.28-3.22 (m, 1H), 3.17-3.11 (m, 1H), 2.43-2.37 (m, 1H), 2.19-2.13 (m, 1H), 1.39 (s, 3H).

LCMS (Method A): 2.490 min, 87.23%, 254.0 nm, MS: ES+ 413.1 (M)

### Step-6:

To a stirred solution of 3-(3-bromo-4-((4-(trifluoromethyl) phenyl) amino) phenyl)-3-methylpyrrolidin-2-one (A5) (0.28 g, 0.677 mmol, 1.0 eq) in MeOH (4 mL) was added KOAc (0.33 g, 3.389 mmol, 5.0 eq) under N₂(g) atmosphere and then PdCl₂(dppf) (0.247 g, 0.338 mmol, 0.5 eq) was added and the resulting mixture was stirred at 100° C. under CO(g) at 40 bar pressure for 16 h. The reaction was monitored on TLC (using 100% EtOAc as mobile phase) which confirmed that the reaction was complete after 16 h of stirring at 100° C. The resulting reaction mixture was directly filtered with celite. The filtrate was dried over Na₂SO₄ and concentrated under reduced pressure to afford 0.42 g crude. The obtained crude material was purified by flash column chromatography using silica (230-400 mesh) as stationary phase (0-0.5% MeOH in DCM) yielding methyl 5-(3-methyl-2-oxopyrrolidin-3-yl)-2-((4-(trifluoromethyl) phenyl) amino) benzoate (A6) (0.085 g, 0.216 mmol, Yield: 31.97%).

¹H NMR (DMSO-d₆, 400 MHz): δ ppm, 9.28 (s, 1H), 7.93 (d, J=2.4 Hz, 1H), 7.86 (br s, 1H), 7.79-7.56 (m, 3H), 7.44 (d, J=8.8 Hz, 1H), 7.33 (d, J=8.4 Hz, 2H), 3.84 (s, 3H), 3.03-3.22 (m, 1H), 3.17-3.09 (m, 1H), 2.42-2.34 (m, 1H), 2.33-2.12 (m, 1H), 1.34 (s, 3H).

LCMS (Method A): 2.545 min, 91.89%,254.0 nm, MS: ES+ 393.0 (M+1)

### Step-7:

To a stirred solution of methyl 5-(3-methyl-2-oxopyrrolidin-3-yl)-2-((4-(trifluoromethyl) phenyl) amino) benzoate (A6) (0.15 g, 0.382 mmol, 1.0 eq), in MeOH (2 mL) was added hydrazine hydrate (1.0 mL, 5.0) at RT then stirred at 90° C. for 4 h. The reaction was monitored on TLC (using MeOH:DCM; 1:9 as mobile phase) which confirmed that the reaction was complete after 4 h of stirring at 90° C. The resulting reaction mixture was concentrated under reduced pressure to afford 0.16 g crude. The obtained crude material was purified by trituration with pentane for yielding 5-(3-methyl-2-oxopyrrolidin-3-yl)-2-((4-(trifluoromethyl) phenyl) amino) benzohydrazide (A7) (0.16 g, 0.407 mmol, Yield: Quantitative).

LCMS (Method A): 1.989 min, 84.56%,254.0 nm, MS: ES+ 393.12 (M+1).

### Step-8:

To a stirred solution of 5-(3-methyl-2-oxopyrrolidin-3-yl)-2-((4-(trifluoromethyl) phenyl) amino) benzohydrazide (A7) (0.15 g, 0.382 mmol, 1.0 eq), in dichloromethane (1.5 mL) was added triethylamine (0.115 g, 1.146 mmol, 3.0 eq) at 0° C. Then acetyl chloride (0.029 g, 0.382 mmol, 1.0 eq) was added at 0° C. and stirred at RT for 4 h. The reaction was monitored on TLC (using MeOH:DCM; 0.5:9.5 as mobile phase) which confirmed that the reaction was complete after 4 h of stirring at RT. The resulting reaction mixture was diluted with H₂O (5 mL) and extracted with DCM (3×10 mL). The combined organic layers were dried over Na₂SO₄ and concentrated under reduced pressure to afford 0.14 g crude. The obtained crude material was purified by trituration with pentane for yielding N′-acetyl-5-(3-methyl-2-oxopyrrolidin-3-yl)-2-((4-(trifluoromethyl) phenyl) amino) benzohydrazide (A8) (0.14 g, 0.322 mmol, Yield: Quantitative).

LCMS (Method A): 1.994 min, 68.01%, 254.0 nm, MS: ES+ 435.3 (M+1).

### Step-9:

To a stirred solution of N′-acetyl-5-(3-methyl-2-oxopyrrolidin-3-yl)-2-((4-(trifluoromethyl) phenyl) amino) benzohydrazide (A8) (0.14 g, 0.322 mmol, 1.0 eq), in dichloromethane (2 mL) was added triethylamine (0.097 g, 0.966 mmol, 3.0 eq) at 0° C. Then TosCl (0.073 g, 0.386 mmol, 1.2 eq) was added at 0° C. and stirred at RT for 3 h. The reaction was monitored on TLC (using MeOH:DCM; 1:9 as mobile phase) which confirmed that the reaction was complete after 3 h of stirring at RT. The resulting reaction mixture was diluted with H₂O (5 mL) and extracted with DCM (3×10 mL). The combined organic layers were dried over Na₂SO₄ and concentrated under reduced pressure to afford 0.18 g crude. The obtained crude material was purified by Prep HPLC purification Method-A yielding 3-methyl-3-(3-(5-methyl-1,3,4-oxadiazol-2-yl)-4-((4-(trifluoromethyl) phenyl) amino) phenyl) pyrrolidin-2-one (Compound 1) (0.025 g, 0.060 mmol, Yield: 18.63%).

¹H NMR (DMSO-d₆, 400 MHz): δ ppm, 9.20 (s, 1H), 7.89 (s, 2H), 7.62 (d, J=8.4 Hz, 2H), 7.56 (s, 2H), 7.35 (d, J=8.4 Hz, 2H), 3.30-3.24 (m, 1H), 3.17-3.13 (m, 1H), 2.59 (s, 3H), 2.45-2.38 (m, 1H), 2.22-2.16 (m, 1H), 1.42 (s, 3H).

LCMS (Method A): 2.350 min, 99.70%,254.0 nm, MS: ES+ 417.1 (M+1).

HPLC (Method B): 8.27 min, 99.87%, 254.0 nm

### Example 2—Synthesis of 3-methyl-3-(3-(1-methyl-1H-imidazol-4-yl)-4-((4-(trifluoromethyl) phenyl) amino) phenyl) pyrrolidin-2-one (Compound 2)

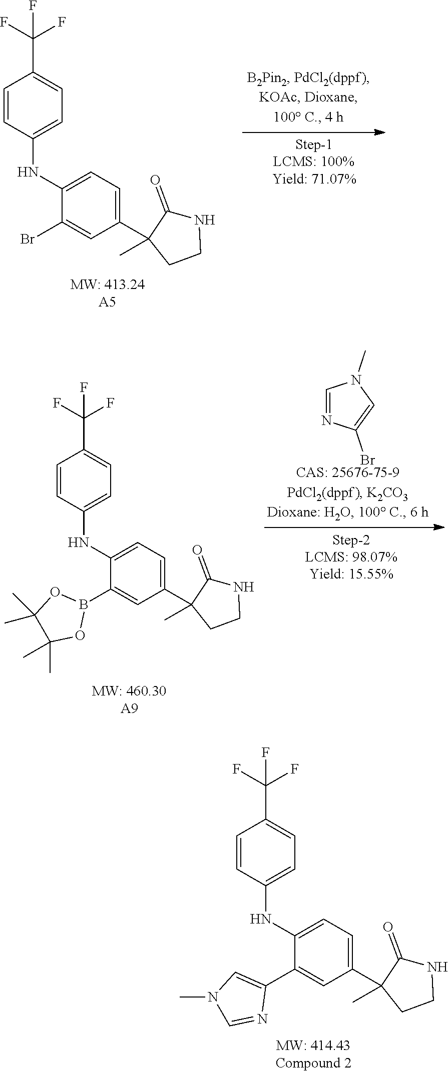

### Step-1:

To a stirred solution of 3-(3-bromo-4-((4-(trifluoromethyl) phenyl) amino) phenyl)-3-methylpyrrolidin-2-one (A5) (1.2 g, 2.903 mmol, 1.0 eq), in 1,4-dioxane (12 mL) was added B₂Pin₂ (1.10 g, 4.355 mmol, 2.0 eq) at rt then potassium acetate (0.56 g, 5.807 mmol, 3.0 eq) was added and purged with N₂(g) for 30 min at RT. Then PdCl₂(dppf) (0.10 g, 1.451 mmol, 0.05 eq) was added and stirred at 100° C. for 4 h. The reaction was monitored on TLC (using EtOAc: Hexane; 7:3 as mobile phase) which confirmed that the reaction was complete after 4 h of stirring at 100° C. The resulting reaction mixture was diluted with H₂O (20 mL) and extracted with EtOAc (3×30 mL). The combined organic layers were dried over Na₂SO₄ and concentrated under reduced pressure to afford 1.4 g crude. The obtained crude material was purified by flash column chromatography using silica (230-400 mesh) as stationary phase (0-80% EtOAc in Hexane) yielding 3-methyl-3-(3-(4,4,5,5-tetramethyl-1,3,2-dioxaborolan-2-yl)-4-((4-(trifluoromethyl) phenyl) amino) phenyl) pyrrolidin-2-one (A9) (0.950 g, 2.065 mmol, Yield: 71.07%).

¹H NMR (DMSO-d₆, 400 MHz): δ ppm, 8.12 (s, 1H), 7.82 (s, 1H), 7.62 (d, J=2.4 Hz, 1H), 7.52-7.47 (m, 3H), 7.29 (d, J=8.4 Hz, 1H), 7.11 (d, J=8.8 Hz, 2H), 3.26-3.212 (m, 1H), 3.14-3.08 (m, 1H), 2.38-2.31 (m, 1H), 2.17-2.10 (m, 1H), 1.38 (s, 3H), 1.25 (s, 12H)

LCMS (Method A): 2.173 min+2.839 min, 58.16+41.84=100.00%, 254.0 nm, MS: ES+ 461.20 (M+1), 379.28 (M−82).

### Step-2:

To a stirred solution of 4-bromo-1-methyl-1H-imidazole (CAS: 25676-75-9) (0.25 g, 1.552 mmol, 1.0 eq), in 1,4-dioxane:H₂O (7:3) (10 mL) was added 3-methyl-3-(3-(4,4,5,5-tetramethyl-1,3,2-dioxaborolan-2-yl)-4-((4-(trifluoromethyl) phenyl) amino) phenyl) pyrrolidin-2-one (A9) (0.85 g, 1.863 mmol, 1.2 eq) at rt then potassium carbonate (0.64 g, 4.658 mmol, 3.0 eq) was added and purged with N₂(g) for 30 min at RT. Then PdCl₂(dppf) (0.056 g, 0.077 mmol, 0.05 eq) was added and stirred at 100° C. for 6 h. The reaction was monitored on TLC (using MeOH:DCM; 0.5:9.5 as mobile phase) which confirmed that the reaction was complete after 6 h of stirring at 100° C. The resulting reaction mixture was diluted with H₂O (20 mL) and extracted with EtOAc (3×30 mL). The combined organic layers were dried over Na₂SO₄ and concentrated under reduced pressure to afford 0.75 g crude. The obtained crude material was purified by flash column chromatography using silica (230-400 mesh) as stationary phase (0-3% MeOH in DCM) yielding 3-methyl-3-(3-(1-methyl-1H-imidazol-4-yl)-4-((4-(trifluoromethyl) phenyl) amino) phenyl) pyrrolidin-2-one (Compound 2) (0.10 g, 0.241 mmol, Yield: 15.55%)

¹H NMR (DMSO-d₆, 400 MHz): δ ppm, 9.82 (s, 1H), 7.80-7.76 (m, 3H), 7.55 (s, 1H), 7.49 (d, J=8.4 Hz, 2H), 7.34 (d, J=8.4 Hz, 1H), 7.23 (dd, J=8.4, 1.6 Hz, 1H), 7.08 (d, J=8.4 Hz, 2H), 3.69 (s, 3H), 3.25-3.22 (m, 1H), 3.17-3.14 (m, 1H), 2.44-2.41 (m, 1H), 2.18-2.14 (m, 1H), 1.42 (s, 3H). Note: one proton merged with DMSO solvent peak

LCMS (Method A): 1.851 min, 98.07%, 254.0 nm, MS: ES+ 415.17 (M+1).

HPLC (Method A): 8.15 min, 97.38%, 254.0 nm

### Example 3—Synthesis of 1-(3-(1-methyl-1H-imidazol-4-yl)-4-((4-(trifluoromethyl)phenyl) amino)benzyl)imidazolidin-2-one (Compound 3)

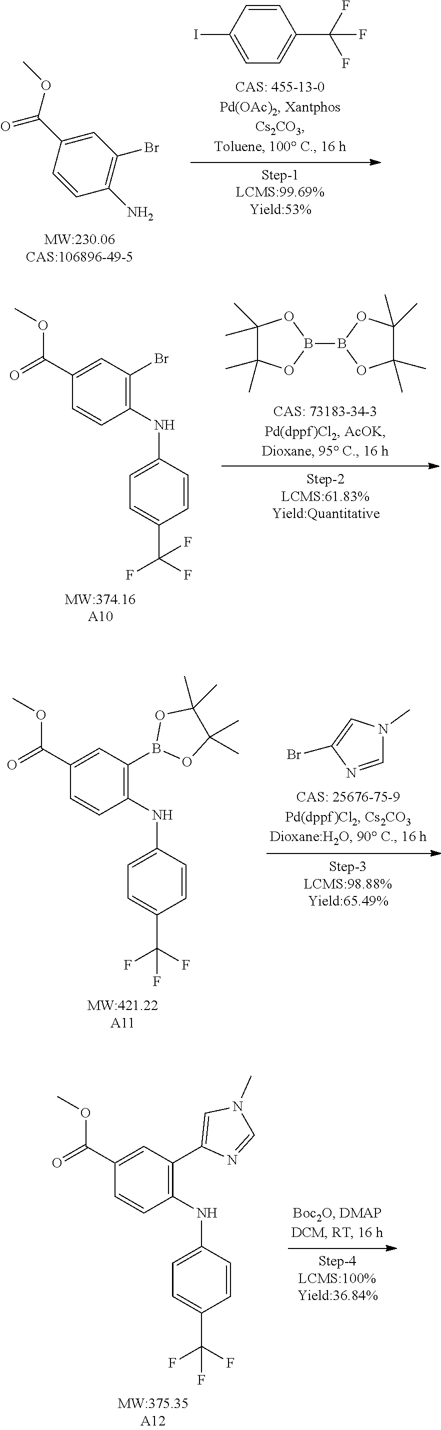

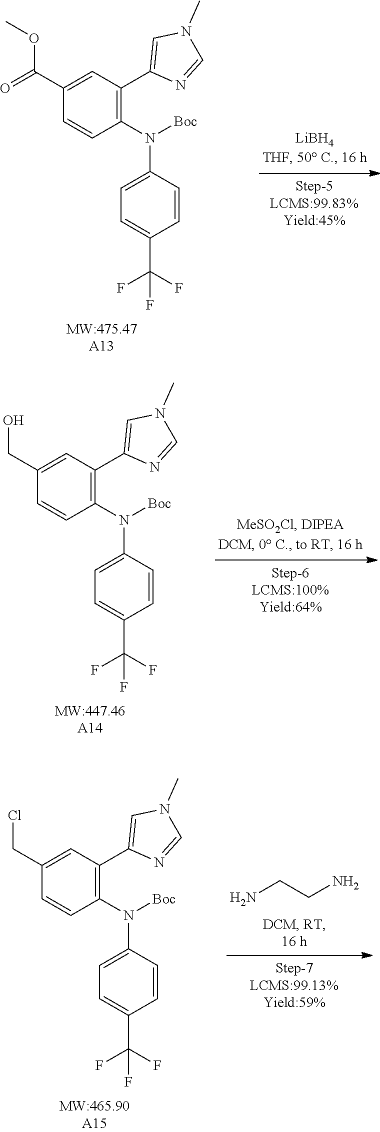

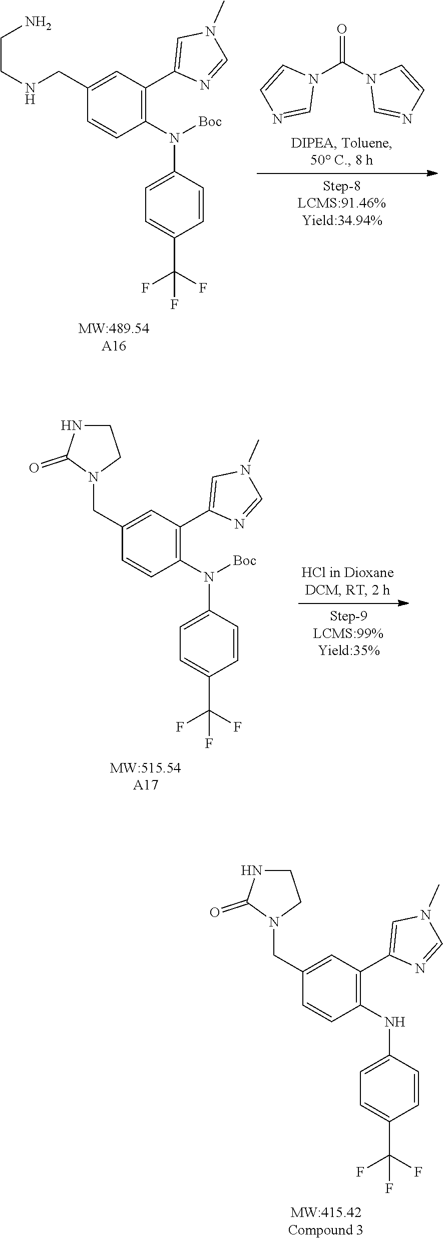

### Step-1:

To a solution of methyl 4-amino-3-bromobenzoate (CAS:106896-49-5) (10.0 g, 43.478 mmol 1.0 eq), CAS: 455-13-0 (11.8 g, 43.478 mmol, 1.0 eq) in Toluene (100 mL) was added cesium carbonate (42 g, 130.43 mmol, 3.0 eq), and the reaction mixture was stirred at RT with N₂ purging for 10 min. Pd(OAc)₂ (0.97 g, 4.34 mmol 0.1 eq) and Xantphos (2.5 g, 4.347 mmol, 0.1 eq) were then added under a nitrogen atmosphere. The reaction mixture was heated at 100° C. with stirring for 16 h. The reaction was monitored on TLC (using EtOAc:Hex; 3:7 as mobile phase). The resulting reaction mixture was quenched with ice-cold water (100 mL) and extracted with EtOAc (3×200 mL). The combined organic layers were dried over Na₂SO₄ and concentrated under reduced pressure to afford 13.0 g crude. The obtained crude material was purified by manual column using silica (60-120 mesh) as stationary phase (eluent 30% ethyl acetate in hexane) yielding methyl 3-bromo-4-((4-(trifluoromethyl) phenyl) amino) benzoate (A10) (8.7 g, 23.26 mmol, Yield: 53%).

¹H NMR (DMSO-d6, 400 MHz): δ ppm, 8.50 (s, 1H), 8.13 (d, J=2.0 Hz, 1H), 7.84 (dd, J=8.4 Hz, 2.0 Hz, 1H), 7.63 (d, J=8.4 Hz, 2H), 7.41 (d, J=8.4 Hz, 1H), 7.32 (d, J=8.4 Hz, 2H), 3.83 (s, 3H)

LCMS (Method A):10.559 min, 99.69%, 210.0 nm, MS: ES+ 375 (M+1)

### Step-2:

To a solution of methyl 3-bromo-4-((4-(trifluoromethyl) phenyl) amino) benzoate (A10) (10.0 g, 43.47 mmol, 1.0 eq), CAS: 73183-34-3 (11.8 g, 43.47 mmol, 1.0 eq) in dioxane (100 mL) was added KOAc (42.0 g, 130.43 mmol, 3.0 eq), then the reaction mixture was stirred at RT with N₂ purging for 10 min. Pd(dppf)Cl₂ (0.97 g, 4.347 mmol 0.1 eq) was then added, under nitrogen atmosphere and the resulting mixture heated at 95° C. for 16 h. The reaction was monitored on TLC (using EtOAc:Hex; 1:9 as mobile phase). The resulting reaction mixture was quenched with ice-cold water (100 mL) and extracted with EtOAc (3×150 mL). The combined organic layers were dried over Na₂SO₄ and concentrated under reduced pressure to afford 12.0 g crude. The obtained crude material was used directly for next step, yielding methyl 3-(4,4,5,5-tetramethyl-1,3,2-dioxaborolan-2-yl)-4-((4-(trifluoromethyl) phenyl) amino) benzoate (A11) (12 g crude, 28.503 mmol, Yield: quantitative)

LCMS (Method A): 3.219 min, 61.83%, 254.0 nm, MS: ES+ 422 (M+1)

### Step-3:

To a solution of methyl 3-(4,4,5,5-tetramethyl-1,3,2-dioxaborolan-2-yl)-4-((4-(trifluoromethyl) phenyl) amino) benzoate (A11) (12 g, 28.50 mmol, 1.0 eq) CAS:25676-75-9 (5.04 g, 31.35 mmol, 1.1 eq) in dioxane:water (8:2) (120 mL) was added cesium carbonate (42.0 g, 57.07 mmol, 2.0 eq), and reaction mixture was stirred at RT with N₂ purging for 10 min. Pd(dppf)Cl₂ (2.0 g, 28.50 mmol 0.1 eq) was then added under a nitrogen atmosphere and the reaction mixture was heated to 90° C. and maintained, with stirring for 16 h. The reaction was monitored on TLC (using EtOAc:Hex; 7:3 as mobile phase). The resulting reaction mixture was quenched with ice-cold water (100 mL) and extracted with EtOAc (3×200 mL). The combined organic layers were dried over Na₂SO₄ and concentrated under reduced pressure to afford 14.0 g crude. The obtained crude material was purified by manual column using silica (60-120 mesh) as stationary phase (eluent 15% Ethyl acetate in hexane) yielding methyl 3-(1-methyl-1H-imidazol-4-yl)-4-((4-(trifluoromethyl) phenyl) amino) benzoate (A12) (7.0 g, 18.66 mmol, Yield: 65.49%).

¹H NMR (DMSO-d6, 400 MHz): δ ppm, 11.03 (s, 1H), 8.23 (d, J=2.0 Hz, 1H), 7.87 (s, 1H), 7.82 (d, J=1.2 Hz, 1H), 7.74 (dd, J=8.4 Hz, 2.0 Hz, 1H), 7.64 (d, J=8.4 Hz, 2H), 7.48 (d, J=8.8 Hz, 1H), 7.36 (d, J=8.4 Hz, 2H), 3.84 (s, 3H), 3.74 (s, 3H). Note: Minor aliphatic impurity observed.

LCMS (Method A):2.174 min, 98.88%, 254.0 nm, MS: ES+ 376 (M+1)

### Step-4:

A solution of methyl 3-(1-methyl-1H-imidazol-4-yl)-4-((4-(trifluoromethyl) phenyl) amino) benzoate (A12) (3.0 g, 8.0 mmol, 1.0 eq), Boc₂O (2.6 g, 12.0 mmol, 1.5 eq), and DMAP (0.39 g, 3.20 mmol, 0.4 eq) in DCM (20 mL) was stirred at RT for 16 h. The reaction was monitored on TLC (using EtOAc:Hex; 1:1 as mobile phase) which confirmed that the reaction was completed after 16 h. The resulting reaction mixture was quenched with water (50 mL) and extracted with EtOAc (3×50 mL). The combined organic layers were dried over Na₂SO₄ and concentrated under reduced pressure to afford 4.0 g crude. The obtained crude material was purified by combi flash using silica (230-400 mesh) as stationary phase (10% Ethyl acetate in hexane) yielding methyl 4-((tert-butoxycarbonyl) (4-(trifluoromethyl) phenyl) amino)-3-(1-methyl-1H-imidazol-4-yl) benzoate (A13) (1.4 g, 2.947 mmol, Yield: 36.84%).

¹H NMR (DMSO-d6, 400 MHz): δ ppm, 8.65 (d, J=2.0 Hz, 1H), 7.81 (dd, J=8.8 Hz, 2.0 Hz, 1H), 7.72 (s, 1H), 7.66 (d, J=8.8 Hz, 2H), 7.39 (d, J=8.8 Hz, 2H), 7.26 (d, J=8.0 Hz, 1H), 7.20 (d, J=1.2 Hz, 1H), 3.89 (s, 3H), 3.63 (s, 3H), 1.15 (s, 9H).

LCMS (Method A): 2.234 min, 100%, 254.0 nm, MS: ES+ 476 (M+1)

### Step-5:

To a solution of methyl 4-((tert-butoxycarbonyl) (4-(trifluoromethyl) phenyl) amino)-3-(1-methyl-1H-imidazol-4-yl) benzoate (A13) (1.4 g, 2.947 mmol, 1.0 eq), in THE (14 mL) at RT was added LiBH₄ (2.9 mL, 2.947 mmol, 1.0 eq). The reaction mixture was heated to 50° C. and stirring was maintained for 16 h. The reaction was monitored on TLC (using EtOAc:Hex; 9:1 as mobile phase) which confirmed that the reaction was completed after 16 h. The resulting reaction mixture was quenched with water (30 mL) and extracted with DCM (3×30 mL). The combined organic layers were dried over Na₂SO₄ and concentrated under reduced pressure to afford 1.6 g crude. The obtained crude material was purified by combi flash using silica (230-400 mesh) as stationary phase (85% Ethyl acetate in hexane) yielding tert-butyl (4-(hydroxymethyl)-2-(1-methyl-1H-imidazol-4-yl) phenyl) (4-(trifluoromethyl) phenyl) carbamate (A14) (0.6 g, 1.342 mmol, Yield: 45%).

¹H NMR (DMSO-d6, 400 MHz): δ ppm, 8.0 (d, J=1.2 Hz, 1H), 7.66-7.62 (m, 3H), 7.37 (d, J 8.4 Hz, 2H), 7.20 (dd, J=8.4 Hz, 2.0 Hz, 1H), 7.08-7.05 (m, 2H), 5.30 (t, J=6.0 Hz, 1H), 4.55 (d, J=6.0 Hz, 2H), 3.61 (s, 3H), 1.17 (s, 9H)

LCMS (Method A): 1.848 min, 99.83%, 254.0 nm, MS: ES+ 448 (M+1)

### Step-6:

To a solution of tert-butyl (4-(hydroxymethyl)-2-(1-methyl-1H-imidazol-4-yl) phenyl) (4-(trifluoromethyl) phenyl) carbamate (A14) (0.6 g, 1.342 mmol, 1.0 eq) in DCM (6 mL) stirred at 0° C., was added DIPEA (0.074 g, 2.684 mmol, 2.0 eq) and MeSO₂Cl (0.183 g, 1.610 mmol, 1.2 eq). The resulting mixture was allowed to warm to RT and stirring maintained for 16 h. The reaction was monitored by TLC (using EtOAc:Hex; 7:3 as mobile phase) which confirmed that the reaction was completed. The resulting reaction mixture was diluted with water (10 mL) and extracted with DCM (3×20 mL) dried over Na₂SO₄ and concentrated under reduced pressure to afford 1.0 g crude. The obtained crude material was purified by CombiFlash® using silica (230-400 mesh) as stationary phase (50% Ethyl acetate in hexane) yielding tert-butyl (4-(chloromethyl)-2-(1-methyl-1H-imidazol-4-yl) phenyl) (4-(trifluoromethyl) phenyl) carbamate (A15) (0.40 g, 0.860 mmol, Yield: 64%)

¹H NMR (DMSO-d6, 400 MHz): δ ppm, 8.11 (d, J=2.4 Hz, 1H), 7.69-7.64 (m, 3H), 7.38 (d, J=8.8 Hz, 2H), 7.31 (dd, J=8.0 Hz, 2.4 Hz, 1H), 7.14-7.12 (m, 2H), 4.84 (s, 2H), 3.62 (s, 3H), 1.17 (s, 9H)

LCMS (Method A): 2.266 min, 100%, 254.0 nm, MS: ES+ 466 (M+1)

### Step-7:

A solution of tert-butyl (4-(chloromethyl)-2-(1-methyl-1H-imidazol-4-yl) phenyl) (4-(trifluoromethyl) phenyl) carbamate (A15) (0.4 g, 0.860 mmol, 1.0 eq) and CAS 107-15-3 (0.051 g, 0.86 mmol, 1.0 eq) in DCM (4.0 mL) was stirred at RT for 16 h. The reaction was monitored by TLC (using DCM:MeOH; 9:1 as mobile phase) which confirmed that the reaction was completed after 16 h of stirring at RT. The resulting reaction mixture was diluted with water (10 mL) and extracted with DCM (3×20 mL). Dried over Na₂SO₄ and concentrated under reduced pressure to afford 0.4 g crude. The obtained crude material was triturated by n-pentane yielding tert-butyl (4-(((2-aminoethyl) amino) methyl)-2-(1-methyl-1H-imidazol-4-yl) phenyl) (4-(trifluoromethyl) phenyl) carbamate (A16) (0.25 g, 0.511 mmol, Yield: 59%).

¹H NMR (DMSO-d6, 400 MHz): δ ppm, 7.98 (s, 1H), 7.65-7.64 (m, 3H), 7.62 (s, 1H), 7.39-7.37 (m, 2H), 7.23-7.21 (m, 1H), 7.09 (s, 1H), 7.051-7.031 (m, 1H), 3.74 (s, 2H), 3.64-3.54 (m, 4H), 3.35-3.30 (m, 1H), 2.65-2.62 (m, 2H), 2.55-2.50 (m, 2H), 1.17 (s, 9H).

LCMS (Method A): 1.50 min, 99.13%, 254.0 nm, MS: ES+ 489 (M+1)

### Step-8:

To a stirred solution of tert-butyl (4-(((2-aminoethyl) amino) methyl)-2-(1-methyl-1H-imidazol-4-yl) phenyl)(4-(trifluoromethyl)phenyl)carbamate (A16) (0.25 g, 0.511 mmol, 1.0 eq) in toluene (2.5 mL) at RT, was added DIPEA (0.074 g, 0.613 mmol, 2.0 eq) and CDI (0.099 g, 0.613 mmol, 1.2 eq). The resulting mixture was heated to 50° C. and stirring maintained for 8 h. The reaction was monitored by TLC (using DCM:MeOH; 9:1 as mobile phase) which confirmed that the reaction was completed after 8 h of stirring at 50° C. The resulting reaction mixture was diluted with water (10 mL) and extracted with EtOAc (3×20 mL) dried over Na₂SO₄ and concentrated under reduced pressure to afford 0.3 g crude. The obtained crude material was purified by combi flash using silica (200-400 mesh) as stationary phase (7% DCM in MeOH) yielding tert-butyl (2-(1-methyl-1H-imidazol-4-yl)-4-((2-oxoimidazolidin-1-yl)methyl)phenyl)(4-(trifluoromethyl) phenyl) carbamate (A17) (0.092 g, 0.178 mmol, Yield: 34.94%).

¹H NMR (DMSO-d6, 400 MHz): δ ppm, 7.92 (d, J=1.6 Hz, 1H), 7.66-7.63 (m, 3H), 7.38 (d, J=8.4 Hz, 2H), 7.14-7.08 (m, 3H), 6.47 (s, 1H), 4.30 (s, 2H), 3.61 (s, 3H), 3.17 (s, 3H), 1.17 (s, 9H).

LCMS (Method A): 1.908 min, 91.46%, 254.0 nm, MS: ES+ 516 (M+1)

### Step-9:

To a stirred solution of tert-butyl (2-(1-methyl-1H-imidazol-4-yl)-4-((2-oxoimidazolidin-1-yl) methyl) phenyl) (4-(trifluoromethyl) phenyl) carbamate (A17) (0.15 g, 0.376 mmol, 1.0 eq), in DCM (1 ml) at RT was added 4 M HCl in dioxane (10 mL). The resulting mixture was stirred at RT for 2 h. The reaction was monitored by TLC (using DCM:MeOH; 9:1 as mobile phase) which confirmed that the reaction was completed after 2 h of stirring at RT. The resulting reaction mixture was basified with sat. NaHCO₃ (5 mL) and extracted with EtOAc (3×10 mL) dried over Na₂SO₄ and concentrated under reduced pressure to afford 0.09 g crude. The obtained crude material was purified by combi flash using silica (200-400 mesh) as stationary phase (5% DCM in MeOH) yielding 1-(3-(1-methyl-1H-imidazol-4-yl)-4-((4-(trifluoromethyl) phenyl) amino) benzyl) imidazolidin-2-one (Compound 3) (0.026 g, 0.062 mmol, Yield: 35%)

¹H NMR (DMSO-d6, 400 MHz): δ ppm, 9.87 (s, 1H), 7.77 (d, J=0.8 Hz, 1H), 7.64 (d, J=2.0 Hz, 1H), 7.50 (d, J=1.2 Hz, 1H), 7.51 (d, J=8.8 Hz, 2H), 7.36 (d, J=8.0 Hz, 1H), 7.10-7.07 (m, 3H), 6.41 (s, 1H), 4.22 (s, 2H), 3.69 (s, 3H), 3.23 (s, 4H).

LCMS (Method A): 1.690 min, 99.78%, 254.0 nm, MS: ES+ 415 (M+1)

HPLC (Method A): 7.77 min, 96.04%, 254.0 nm

### Example 4—Synthesis of 1-(3-(1-methyl-1H-imidazol-4-yl)-4-((4-(trifluoromethyl) phenyl) amino) benzyl) imidazolidine-2,4-dione (Compound 4)

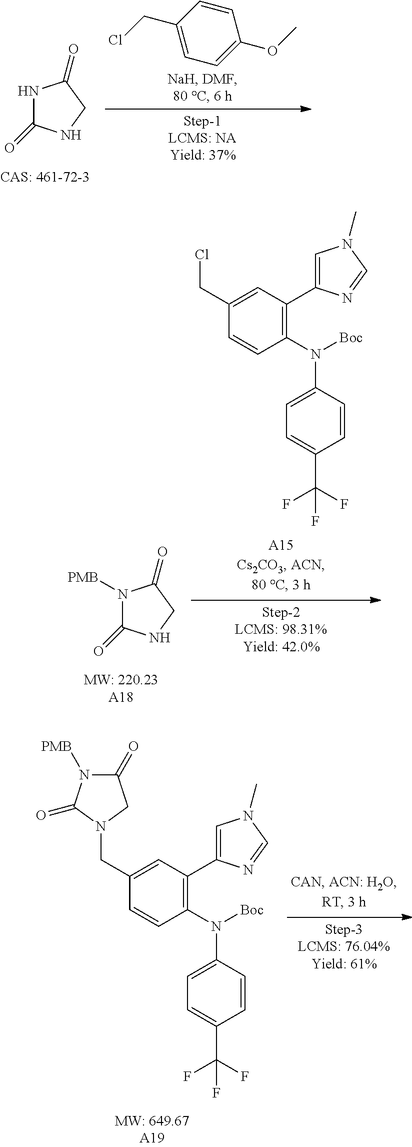

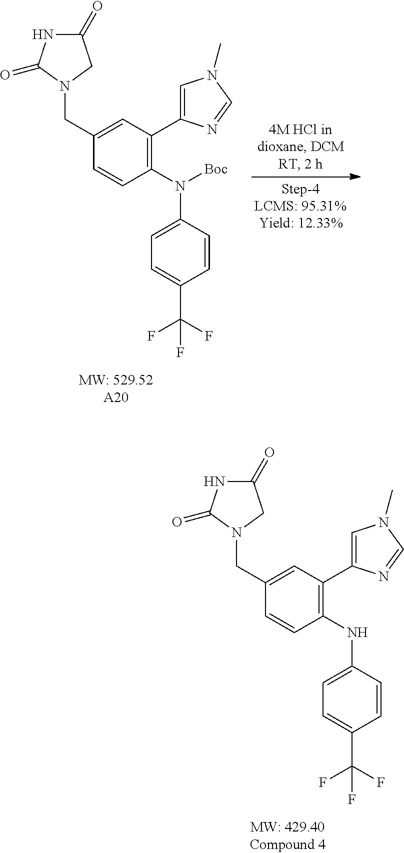 [SMILES: Cn1cnc(-c2cc(CN3CC(=O)NC3=O)ccc2N(C(=O)OC(C)(C)C)c2ccc(C(F)(F)F)cc2)c1 ; Cn1cnc(-c2cc(CN3CC(=O)NC3=O)ccc2Nc2ccc(C(F)(F)F)cc2)c1]

### Step-1:

To a stirred solution of imidazolidine-2,4-dione CAS 461-72-3 (0.3, 3.00 mmol, 1.0 eq), CAS 824-94-2 (0.46 g, 3.00 mmol, 1.0 eq) in DMF (2 mL) at RT, was added NaH (0.086 g, 6.00 mmol, 2.0 eq). The resulting mixture was heated to 80° C. and stirring was maintained for 6 h. The reaction was monitored on TLC (using EtOAc:Hex; 7:3 as mobile phase) which confirmed that the reaction was completed after 6 h. The resulting reaction mixture was quenched with water (5 mL) and extracted with EtOAc (3×25 mL). The combined organic layers were dried over Na₂SO₄ and concentrated under reduced pressure to afford 0.35 g crude. The obtained crude material was purified by flash column chromatography using silica (230-400 mesh) as stationary phase (50% EA in Hex) Yielding 3-(4-methoxybenzyl) imidazolidine-2,4-dione (A18) (0.25 g, 1.136 mmol, Yield: 37%)

¹H NMR (DMSO-d6, 400 MHz): δ ppm, 8.10 (s, 1H), 7.23-7.19 (m, 2H), 6.88 (d, J=8.8 Hz, 2H), 4.44-4.40 (m, 2H), 3.95 (s, 2H), 3.72 (s, 3H).

### Step-2:

To a solution of tert-butyl (4-(chloromethyl)-2-(1-methyl-1H-imidazol-4-yl) phenyl) (4-(trifluoromethyl) phenyl) carbamate (A15) (0.3 g, 0.645 mmol, 1.0 eq) and 3-(4-methoxybenzyl) imidazolidine-2,4-dione (A18) (0.212 g, 0.967 mmol, 1.5 eq) in ACN (3 mL) was added Cs₂CO₃ (0.524 g, 1.612 mmol, 2.5 eq). The reaction mixture was heated to 80° C. and stirring maintained for 3 h. The reaction was monitored on TLC (using EA:HEX; 8:2 as mobile phase) which confirmed that the reaction was completed after 3 h. The resulting reaction mixture was quenched with ice-cold water (5 mL) and extracted with EtOAc (3×25 mL). The combined organic layers were dried over Na₂SO₄ and concentrated under reduced pressure to afford 0.3 g crude. The obtained crude material was purified by flash column chromatography using silica (230-400 mesh) as stationary phase (80% EA in HEX) yielding tert-butyl (4-((3-(4-methoxybenzyl)-2,4-dioxoimidazolidin-1-yl) methyl)-2-(1-methyl-1H-imidazol-4-yl) phenyl) (4-(trifluoromethyl) phenyl) carbamate (A19) (0.18 g, 0.277 mmol, Yield: 42%).

¹H NMR (DMSO-d6, 400 MHz): δ ppm, 7.93 (d, J=2.0 Hz, 1H), 7.67 (s, 1H), 7.64-7.62 (d, J 8.8 Hz, 2H), 7.39 (d, J=8.4 Hz, 2H), 7.24 (d, J=8.8 Hz, 2H), 7.16 (dd, J=8.0 Hz, 2.4 Hz, 1H), 7.11-7.09 (m, 2H), 6.88 (d, J=8.4 Hz, 2H), 4.58 (s, 2H), 4.52 (s, 2H), 3.96 (s, 2H), 3.73 (s, 3H), 3.62 (s, 3H), 1.24 (s, 9H).

LCMS (Method A): 2.259 min, 98.94%, 254.0 nm, MS: ES+ 649 (M+1)

### Step-3:

To a solution tert-butyl (4-((3-(4-methoxybenzyl)-2,4-dioxoimidazolidin-1-yl) methyl)-2-(1-methyl-1H-imidazol-4-yl) phenyl) (4-(trifluoromethyl) phenyl) carbamate (A19) (0.18 g, 0.277 mmol, 1.0 eq) in ACN:H₂O (1.8 mL) was added CAN (0.151 g, 0.277 mmol, 1.0 eq). The resulting mixture was stirred for 3 h at room temperature. The reaction was monitored on TLC (using EA:HEX; 9:1 as mobile phase) which confirmed that the reaction was completed after 3 h. The resulting reaction mixture was quenched with ice-cold water (5 mL) and extracted with EtOAc (3×50 mL). The combined organic layers were dried over Na₂SO₄ and concentrated under reduced pressure to afford 0.2 g crude. The obtained crude material was purified by flash column chromatography using silica (230-400 mesh) as stationary phase (5% DCM in MeOH) yielding tert-butyl (4-((2,4-dioxoimidazolidin-1-yl) methyl)-2-(1-methyl-1H-imidazol-4-yl) phenyl) (4-(trifluoromethyl) phenyl) carbamate (A20) (0.09 g, 0.170 mmol, Yield: 61%).

¹H NMR (DMSO-d6, 400 MHz): δ ppm, 10.95 (brs, 1H) 9.87 (s, 1H), 7.87 (d, J=8.0 Hz, 1H), 7.67-7.65 (m, 3H), 7.44-7.26 (m, 4H), 7.13 (d, J=8.8 Hz, 1H), 4.53 (s, 2H), 3.91-3.87 (m, 2H), 3.78 (brs, 3H), 1.22 (s, 9H).

LCMS (Method A): 1.898 min, 62.16%, 254.0 nm, MS: ES+ 529 (M+1)

### Step-4:

To a stirred solution of tert-butyl (4-((2,4-dioxoimidazolidin-1-yl) methyl)-2-(1-methyl-1H-imidazol-4-yl) phenyl) (4-(trifluoromethyl) phenyl) carbamate (A20) (0.09 g, 0.170 mmol, 1.0 eq), in DCM (1 ml) at RT was added 4 M HCl in dioxane (10 mL) and the reaction mixture was stirred at RT for 2 h. The reaction was monitored by TLC (using DCM:MeOH; 9:1 as mobile phase) which confirmed that the reaction was completed after 2 h of stirring at RT. The resulting reaction mixture was basified with sat. NaHCO₃ (5 mL) and extracted with EtOAc (3×25 mL), dried over Na₂SO₄ and concentrated under reduced pressure to afford 0.09 g. The obtained crude material was purified by flash column chromatography using silica (230-400 mesh) as stationary phase; desired product eluted in 7% DCM in MeOH) yielding 1-(3-(1-methyl-1H-imidazol-4-yl)-4-((4-(trifluoromethyl) phenyl) amino) benzyl) imidazolidine-2,4-dione (Compound 4) (0.0091 g, 0.0212 mmol, Yield: 12.33%)

¹H NMR (DMSO-d₆, 400 MHz): δ ppm, 10.73 (s, 1H), 9.98 (s, 1H), 7.78 (s, 1H), 7.64 (s, 1H), 7.59 (s, 1H), 7.51 (d, J=8.4 Hz, 2H), 7.37 (d, J=8.4 Hz, 1H), 7.12-7.10 (m, 3H), 4.42 (s, 2H), 3.87 (s, 2H), 3.70 (s, 3H).

LCMS (Method A):1.710 min, 95.31%, 254.0 nm, MS: ES+ 429 (M+1)

HPLC (Method A):5.95 min, 97.18%, 210.0 nm

### Example 5—Chiral Separation of 3-methyl-3-(3-(1-methyl-1H-imidazol-4-yl)-4-((4-(trifluoromethyl)phenyl)amino)phenyl)pyrrolidin-2-one (Compounds 5 and 6)

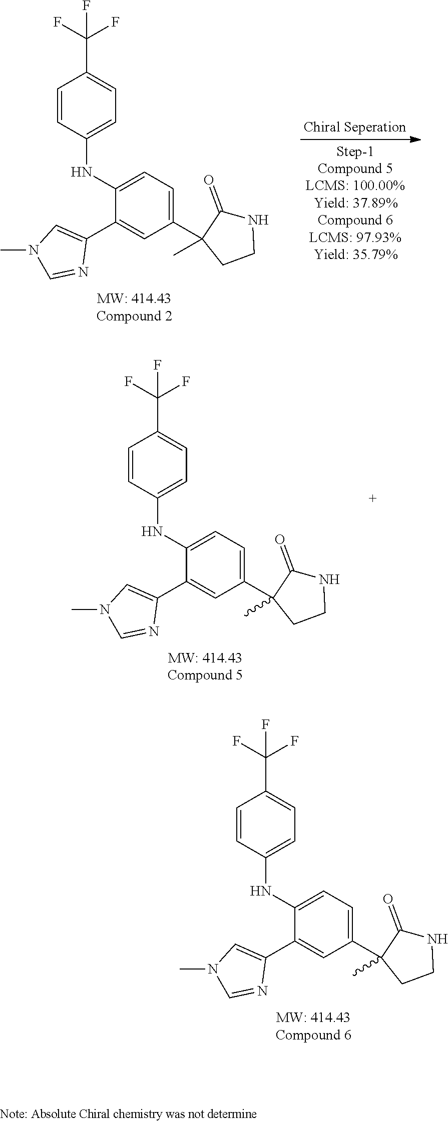

Compound 2 (0.096 g) racemic was subjected to chiral SFC purification on (CHIRALPAK IA 250×50 mm 5 um) column where 2 peaks were separated Compound 2

Peak-1 (Compound 5) (0.036 g, Yield=37.89%), and Compound 2 Peak-2 (Compound 6) (0.034 g Yield=35.79%)

COLUMN ID: CHIRALPAK IA 250×50 mm 5 um

MOBILE PHASE A: LIQ. CO2

MOBILE PHASE B: 0.1% M. NH3 IN MEOH-ACN (50-50)

FLOW RATE (ML/MIN): 160

INSTRUMENT ID: WATERS SFC 350 WITH 2489 UV Detector

METHOD: TIME: FLOW: % A: % B (0.01:160:60:40), (12.00:160:60:40)

### Compound 5

¹H NMR (DMSO-d₆, 400 MHz): δ ppm, 9.81 (s, 1H), 7.80 (s, 1H), 7.77-7.76 (m, 2H), 7.55 (s, 1H), 7.49 (d, J=8.8 Hz, 2H), 7.34 (d, J=8.4 Hz, 1H), 7.23 (dd, J=8.4, 2.0 Hz, 1H), 7.08 (d, J 8.4 Hz, 2H), 3.69 (s, 3H), 3.26-3.17 (m, 2H), 2.42-2.41 (m, 1H), 2.18-2.08 (m, 1H), 1.42 (s, 3H).

LCMS (Method A): 1.807 min, 100.0%, 254.0 nm, MS: ES+ 415.17 (M+1)

HPLC (Method A): 8.03 min, 99.48%, 254.0 nm

CHIRAL HPLC: 3.19 min, 100.00%, 310.0 nm

### Compound 6

¹H NMR (DMSO-d₆, 400 MHz): δ ppm, 9.82 (s, 1H), 7.81 (s, 1H), 7.77-7.76 (m, 2H), 7.55 (s, 1H), 7.49 (d, J=8.8 Hz, 2H), 7.34 (d, J=8.4 Hz, 1H), 7.23 (dd, J=8.0, 2.0 Hz, 1H), 7.08 (d, J=8.4 Hz, 2H), 3.69 (s, 3H), 3.26-3.14 (m, 2H), 2.41-2.33 (m, 1H), 2.18-2.12 (m, 1H), 1.42 (s, 3H).

LCMS (Method A): 1.812 min, 97.93%,254.0 nm, MS: ES+ 415.17 (M+1)

HPLC (Method A): 8.03 min, 97.26%, 254.0 nm

CHIRAL HPLC: 3.68 min, 100.00%, 310.0 nm

### Example 6—Synthesis of 3-methyl-3-(3-(5-methyl-1,3,4-oxadiazol-2-yl)-4-((4-(trifluoromethyl) phenyl) amino) phenyl) pyrrolidin-2-one (Compounds 7 and 8)

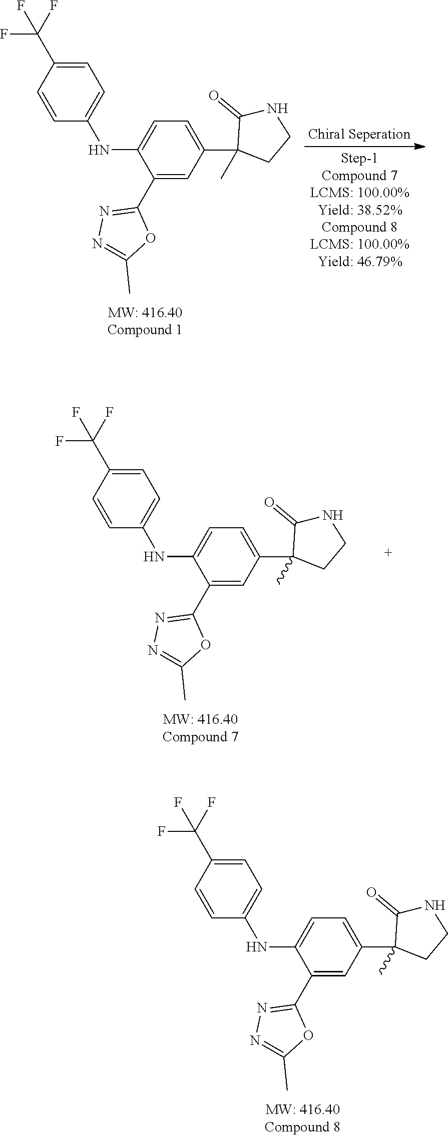

Compound 1 (0.2 g) racemic was subjected to chiral SFC purification on (CHIRALPAK IA 250×50 mm 5 μm) column where 2 peaks were separated Compound 1

Peak-1 (Compound 7) (0.040 g, Yield=38.52%), and Compound 1 Peak-2 (Compound 8) (0.045 g, Yield=46.79%)

COLUMN ID: CHIRALPAK IA 250×50 mm 5 um

MOBILE PHASE A: LIQ. CO2

MOBILE PHASE B: 0.1% M. NH3 IN MEOH-ACN (50-50)

FLOW RATE (ML/MIN): 160

INSTRUMENT ID: WATERS SFC 350 WITH 2489 UV Detector

METHOD: TIME: FLOW: % A: % B (0.01:160:55:45),(16.00:160:55:45)

Input Quantity: 0.2 g.

Output Quantity: Compound 7=0.040 g (% Yield=38.52%) and Compound 8=0.045 g (% Yield=46.79%)

### Compound 7:

¹H NMR (DMSO-d₆, 400 MHz): δ ppm, 9.20 (s, 1H), 7.90-7.89 (m, 2H), 7.62 (d, J=8.8 Hz, 2H), 7.56 (d, J=2.4 Hz, 2H), 7.35 (d, J=8.8 Hz, 2H), 3.26-3.24 (m, 1H), 3.17-3.12 (m, 1H), 2.59 (s, 3H), 2.44-2.38 (m, 1H), 2.22-2.16 (m, 1H), 1.42 (s, 3H)

¹H NMR (MeOD, 400 MHz): δ ppm), 7.99 (d, J=2.0 Hz, 1H), 7.63-7.54 (m, 4H), 7.40 (d, J 8.4 Hz, 2H), 3.47-3.42 (m, 2H), 2.66 (s, 3H), 2.58-2.52 (m, 1H), 2.36-2.30 (m, 1H), 1.63 (s, 3H), 1H merged with MeOD moisture which is clearly visible in DMSO NMR

LCMS (Method A): 2.344 min, 100.00%, 254.0 nm, MS: ES+ 417.38 (M+1)

HPLC (Method B): 8.33 min, 99.60%, 330.0 nm

CHIRAL HPLC: 4.38 min, 100.00%, 300.0 nm

### Compound 8:

¹H NMR (DMSO-d₆, 400 MHz): δ ppm, 9.20 (s, 1H), 7.90-7.89 (m, 2H), 7.62 (d, J=8.4 Hz, 2H), 7.56 (s, 2H), 7.35 (d, J=8.4 Hz, 2H), 3.27-3.24 (m, 1H), 3.17-3.11 (m, 1H), 2.59 (s, 3H), 2.44-2.38 (m, 1H), 2.22-2.16 (m, 1H), 1.42 (s, 3H).

¹H NMR (MeOD, 400 MHz): δ ppm), 7.96 (s, 1H), 7.60-7.54 (m, 4H), 7.37 (d, J=8.4 Hz, 2H), 2.60 (s, 3H), 2.55-2.49 (m, 1H), 2.34-2.27 (m, 1H), 1.55 (s, 3H).

LCMS (Method A): 2.329 min, 98.58%,254.0 nm, MS: ES+ 417.43 (M+1)

HPLC (Method B): 8.32 min, 99.74%, 235.0 nm

CHIRAL HPLC: 3.65 min, 100%, 300.0 nm

### Example 7—Synthesis of 5-methyl-5-(3-(1-methyl-1H-imidazol-4-yl)-4-((4-(trifluoromethyl) phenyl) amino) phenyl) imidazolidine-2,4-dione (Compound 9)

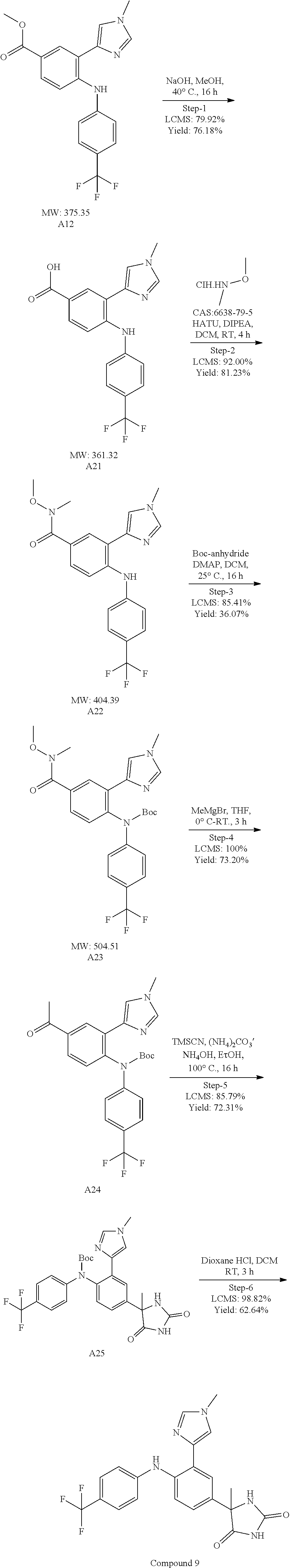

### Step-1:

To a stirred solution of (A12) (1.5 g, 3.99 mmol, 1.0 eq), in methanol (15 mL) was added NaOH solution (0.32 g, 7.99 mmol, 2.0 eq) at 25° C. Then RM was heated to 40° C. and was maintained for 16 h at 40° C. The reaction was monitored by TLC (using 70% EtOAc in hexane as mobile phase) which confirmed that the reaction was completed after 16 h of stirring at 40° C. The resulting reaction mixture was purified by trituration with DCM and pentane to afford 3-(1-methyl-1H-imidazol-4-yl)-4-((4-(trifluoromethyl) phenyl) amino) benzoic acid (A21) (1.1 g, 3.04 mmol, Yield: 76.18%)

¹H NMR (DMSO-d6, 400 MHz): δ ppm, 12.70 (brs, 1H), 10.22 (brs, 1H), 8.31 (brs, 1H), 8.17 (s, 1H), 7.86 (s, 1H), 7.80 (d, J=8.4 Hz, 1H), 7.62 (d, J=8.4 Hz, 2H), 7.52-7.47 (m, 1H), 7.31-7.21 (m, 2H) 3.78 (s, 3H).

LCMS (Method-A): 1.79, 79.92%, 254 nm, MS: ES+ 362 (M), (M+1)

### Step-2:

To a solution of (A21) (1.1 g, 3.04 mmol, 1.0 eq), in DCM (10 mL), was added DIPEA (1.17 g, 9.14 mmol, 3.0 eq) and HATU (2.31 g, 6.09 mmol, 2.0 eq) respectively at 0° C. Then to the reaction mixture was added CAS No. 6638-79-5 (0.32 g, 3.35 mmol, 1.1 eq) at 0° C. The reaction mixture was allowed to RT and stirred for 4 h. The reaction was monitored by TLC (using 70% EtOAc in hexane as mobile phase) which confirmed that the reaction was completed after 4 h of stirring at RT. The resulting reaction mixture was extracted with DCM (3×100 mL). The combined organic layers were dried over Na₂SO₄ and concentrated under reduced pressure to afford 1.5 g crude. The obtained crude material was purified by manual column chromatography using silica (100-200 mesh) as stationary phase (50% EtOAc in hexane as mobile phase) to yield N-methoxy-N-methyl-3-(1-methyl-1H-imidazol-4-yl)-4-((4-(trifluoromethyl) phenyl) amino) benzamide (A22) (1.0 g, 2.47 mmol, Yield: 81.23%)

¹H NMR (DMSO-d6, 400 MHz): δ ppm 10.72 (s, 1H), 7.98 (s, 1H), 7.85 (s, 1H), 7.72 (s, 1H), 7.60 (d, J=8.4 Hz, 2H), 7.49-7.36 (m, 2H), 7.29 (d, J=8.4 Hz, 2H), 3.73 (s, 3H), 3.61 (s, 3H), 3.27 (s, 3H)

LCMS (Method A): 1.816, 92.00%, 254.0 nm, MS: ES+ 405 (M+1).

### Step-3:

To a stirred solution of N-methoxy-N-methyl-3-(1-methyl-1H-imidazol-4-yl)-4-((4-(trifluoromethyl) phenyl) amino) benzamide (A22) (1.0 g, 2.47 mmol, 1.0 eq) in DCM at RT was added (BOC)₂O (0.80 g, 3.70 mmol, 1.5 eq) and DMAP (0.12 g, 0.98 mmol, 0.4 eq), under a nitrogen atmosphere. The reaction mixture was stirred for 16 h. TLC shows completion of reaction (70% EtOAc in hexane as mobile phase). The resulting reaction mixture was extracted with DCM (3×100 mL). The combined organic layers were dried over Na₂SO₄ and concentrated under reduced pressure to afford 1.8 g crude. The obtained crude material was purified by manual column using silica (100-200 mesh) as stationary phase (using 50% EtOAc in hexane as mobile phase) to yield tert-butyl (4-(methoxy(methyl)carbamoyl)-2-(1-methyl-1H-imidazol-4-yl) phenyl) (4-(trifluoromethyl) phenyl) carbamate (A23) (0.45 g, 0.89 mmol, Yield: 36.07%)

¹H NMR (DMSO-d6, 400 MHz): δ ppm, 8.25 (d, J=2.0 Hz, 1H), 7.72-7.65 (m, 3H), 7.60 (d, J 8.8 Hz, 1H), 7.47-7.41 (m, 2H), 7.18-7.17 (m, 2H), 3.66 (s, 3H), 3.62 (s, 3H), 3.28 (s, 3H), 1.16 (s, 9H)

LCMS (Method A):2.032 min, 85.41%, 254.0 nm, MS: ES+ 505 (M+1)

### Step-4:

To a solution of tert-butyl (4-(methoxy(methyl)carbamoyl)-2-(1-methyl-1H-imidazol-4-yl) phenyl) (4-(trifluoromethyl) phenyl) carbamate (A23) (0.45 g, 0.89 mmol, 1.0 eq), in THF, was added MeMgBr in THE (1.78 mL, 2.0 eq), at 0° C. Then reaction mixture was allowed to warm to RT and was stirred for 3 h. The reaction was monitored on TLC (using 50% EtOAc in hexane as mobile phase). Then extracted with EA (3×100 mL). The combined organic layers were dried over Na₂SO₄ and concentrated under reduced pressure to afford 0.7 g crude. The obtained crude material was purified by manual column using silica (100-200 mesh) desired product eluted (15-20% EtOAc in hexane as mobile phase) to yield tert-butyl(4-acetyl-2-(1-methyl-1H-imidazol-4-yl) phenyl) (4-(trifluoromethyl) phenyl) carbamate (A24) (0.30 g, 0.653 mmol, Yield: 73.20%)

¹H NMR (DMSO-d6, 400 MHz): δ ppm, 8.58 (d, J=2.0 Hz, 1H), 7.83 (dd, J=6.0 Hz, 1H), 7.72 (s, 1H), 7.66 (d, J=8.8 Hz, 2H), 7.40 (d, J=8.4 Hz, 2H), 7.29-7.23 (m, 2H), 3.64 (s, 3H), 2.19 (s, 3H), 1.64 (s, 9H).

Note: Impurity peaks observed in ¹H NMR.

LCMS (Method A): 2.115, 100%, 254 nm, MS: ES+ 460 (M+1)

### Step-5:

To a stirred solution of tert-butyl (4-acetyl-2-(1-methyl-1H-imidazol-4-yl) phenyl) (4-(trifluoromethyl) phenyl) carbamate (A24) (0.30 g, 0.653 mmol, 1.0 eq), in ethanol (6 ml) was added ammonium carbonate (0.25 g, 2.61 mmol, 4.0 eq), followed by addition of TMSCN (3 ml) and ammonium hydroxide (6 ml) solution at RT. Then the reaction mixture was heated up to 100° C. and was stirred for 16 h. The reaction was monitored on TLC (using 70% EtOAc in hexane as mobile phase). The resulting reaction mixture was extracted with EtOAc (3×100 mL). The combined organic layers were dried over Na₂SO₄ and concentrated under reduced pressure to afford 0.7 g crude. The obtained crude material was purified by manual column using silica (100-200 mesh) as stationary phase (using 50% EtOAc in hexane as mobile phase) to yielding tert-butyl (2-(1-methyl-1H-imidazol-4-yl)-4-(4-methyl-2,5-dioxoimidazolidin-4-yl) phenyl) (4-(trifluoromethyl) phenyl) carbamate (A25) 0.25 g, 0.472 mmol, Yield: 73.21%)

¹H NMR (DMSO-d6, 400 MHz): δ ppm, 8.71 (d, J=6.0 Hz, 1H), 8.16 (d, J=2.4 Hz, 1H), 8.07 (s, 1H), 7.97 (dd, J=12.0 Hz, 1H), 7.69 (s, 1H), 7.64 (d, J=6.8 Hz, 2H), 7.36 (d, J=6.8 Hz, 2H), 7.16-7.11 (m, 2H), 3.62 (s, 3H), 1.99 (s, 3H), 1.35 (s, 9H).

LCMS (Method A): 1.878 min, 85.79%, 243.0 nm, MS: ES+ 530 (M+1)

### Step-6:

To a solution tert-butyl (2-(1-methyl-1H-imidazol-4-yl)-4-(4-methyl-2,5-dioxoimidazolidin-4-yl)phenyl)(4-(trifluoromethyl)phenyl)carbamate (A25) (0.25 g, 0.472 mmol, 1.0 eq) in 2.5 ml DCM was added HCl. Dioxane (2.49 ml, 5.0 eq) at RT and stirred at 3 hrs. The reaction was monitored on TLC (using 70% EtOAc in hexane as mobile phase). The RM was concentrated under reduced pressure and the crude material was basified by NaHCO₃ solution and was extracted with EtOAc (3×50 mL). The combined organic layers were dried over Na₂SO₄ and concentrated under reduced pressure to afford 0.35 g crude. The crude material was purified by trituration by DCM and pentane solution to obtain 5-methyl-5-(3-(1-methyl-1H-imidazol-4-yl)-4-((4-(trifluoromethyl) phenyl) amino) phenyl) imidazolidine-2,4-dione (Compound 9) (0.127 g, 0.295 mmol, Yield: 62.64%).

¹H NMR (DMSO-d6, 400 MHz): δ ppm, 10.76 (s, 1H), 9.92 (s, 1H), 8.55 (s, 1H), 7.83 (d, J=2.0 Hz, 1H), 7.79 (s, 1H), 7.58 (s, 1H), 7.51 (d, J=8.8 Hz, 2H), 7.41 (d, J=8.4, 1H), 7.28 (dd, J=6.0 Hz, 1H), 7.11 (d, J=8.4, 2H), 3.71 (s, 3H), 1.68 (s, 3H);

LCMS (Method A): 1.64 min, 98.82%, 296.0 nm, MS: ES+ 430 (M+1)

HPLC (Method A): 5.15 min, 98.94%, 210 nm,

### Example 8—Synthesis of (3-methyl-3-(3-(oxazol-2-yl)-4-((4-(trifluoromethyl) phenyl) amino) phenyl) pyrrolidin-2-one (Compound 10)

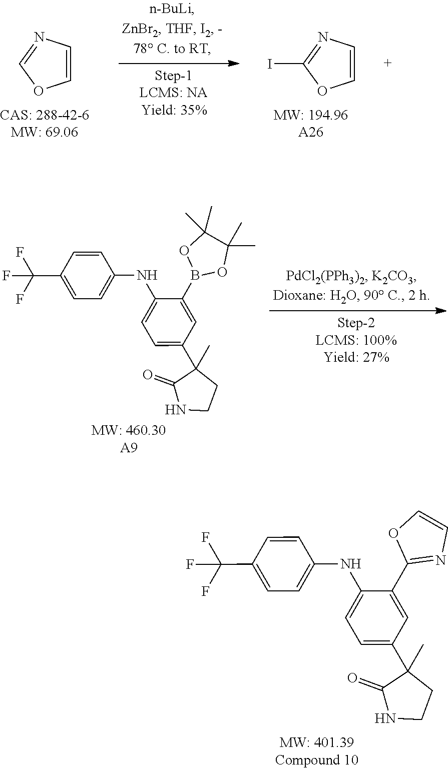 [SMILES: c1cocn1 ; Ic1ncco1 ; CC1(c2ccc(Nc3ccc(C(F)(F)F)cc3)c(B3OC(C)(C)C(C)(C)O3)c2)CCNC1=O ; CC1(c2ccc(Nc3ccc(C(F)(F)F)cc3)c(-c3ncco3)c2)CCNC1=O]

### Step-1:

To a stirred solution of oxazole (2.0 g, 28.9 mmol, 1.0 eq) in THE (20 mL) was prepared in 50 mL of single neck RB at −78° C. To this reaction solution, n-BuLi (21.67 ml, 34.6 mmol, 1.2 eq) was added dropwise followed by addition of ZnBr₂. The resulting mixture was stirred at −78° C. for 1 h, then a solution of iodine in THE was added in reaction mixture and stirred at RT for 30 min. The reaction was monitored by TLC (using EA:Hexane; 2.0:8.0 as mobile phase) which confirmed that the reaction was completed after 30 min of stirring at RT. The resulting reaction mixture was quenched with NH₄Cl solution (20 mL) and extracted with DCM (3×20 mL). The combined organic layers were dried over Na₂SO₄ and concentrated under reduced pressure to afford 2.5 g crude. The obtained crude material was purified by flash column chromatography using silica (230-400 mesh) as stationary phase (5% Ethyl acetate in hexane) to yielding as a 2-iodooxazole (A26) (2.0 g, 10.30 mmol, Yield: 35%)

¹H NMR (DMSO-d6, 400 MHz): δ ppm 8.25 (s, 1H), 7.23 (s, 1H).

### Step-2:

In a 30 mL reactor, a stirred solution of 2-iodooxazole (A26) (0.25 g, 0.54 mmol, 1.0 eq) in dioxane (2 mL) was added (A9) (0.126 g, 0.65 mmol, 1.2 eq) followed by K₂CO₃ (0.22 g, 1.62 mmol, 3 eq) and water under N₂ atm. PdCl₂(dppf) (0.019 g, 0.02 mmol, 0.05 eq) was added. The reactor was sealed and the reaction stirred at 90° C. for 2 h. The reaction was monitored by TLC (using EA:Hexane; 7.0:3.0 as mobile phase) which confirmed that the reaction got completed. The resulting reaction mixture was quenched with water (10 mL) and extracted with EA (3×10 mL). The combined organic layers were dried over Na₂SO₄ and concentrated under reduced pressure to afford 0.3 g crude. The obtained crude material was purified by Prep HPLC purification Method-A to yield as a (3-methyl-3-(3-(oxazol-2-yl)-4-((4-(trifluoromethyl) phenyl) amino) phenyl) pyrrolidin-2-one (Compound 10) (0.03 g, 0.017 mmol, Yield: 27%)

¹H NMR (DMSO-d6, 400 MHz): δ ppm 9.92 (s, 1H), 8.28 (s, 1H), 8.01 (d, J=2.0 Hz, 1H), 7.87 (s, 1H), 7.62 (d, J=8.8 Hz, 2H), 7.55-7.49 (m, 3H), 7.37 (d, J=8.4 Hz, 2H), 3.29-3.14 (m, 2H), 2.22-2.15 (m, 1H), 1.42 (s, 3H). Note: 1H merged with DMSO solvent peak, which is clearly visible in MeOD NMR

¹H NMR (MeOD, 400 MHz): δ ppm 10.17 (s, 1H), 8.09 (d, J=1.6 Hz, 1H), 7.99 (s, 1H), 7.61-7.56 (m, 3H), 7.48 (d, J=8.8 Hz, 1H), 7.40-7.38 (m, 3H), 3.44-3.32 (m, 1H), 2.58-2.52 (m, 1H), 2.36-2.31 (m, 1H), 1.58 (s, 3H).

LCMS: 2.63 min, 100%, 241.0 nm, MS: ES+ 402.2 (M+1)

HPLC: 9.26 min, 100%, 210 nm

### Example 9—Synthesis of 3-methyl-3-(3-(2-methyl-2H-tetrazol-5-yl)-4-((4-(trifluoromethyl) phenyl) amino) phenyl) pyrrolidin-2-one (Compound 11)

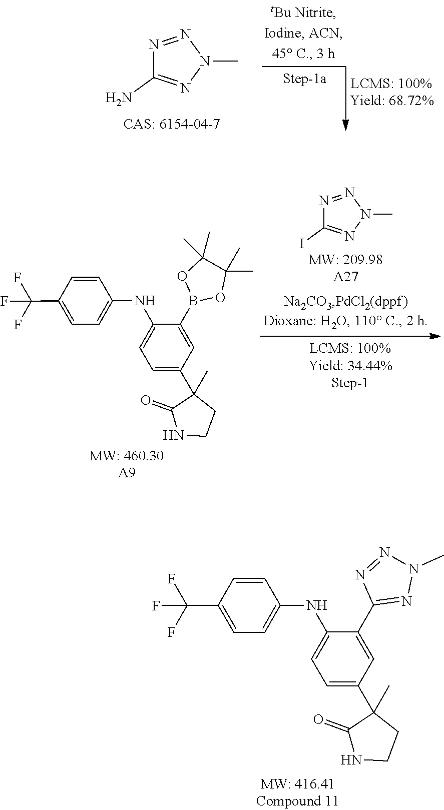

### Step-1a:

In a 10 mL glass vial, a stirred solution of CAS: 6154-04-7 (0.1 g, 1.009 mmol, 1.0 eq) in ACN (2.5 mL) were prepared at room temperature. To this reaction solution, iodine (0.256 g, 1.009 mmol, 1.0 eq) was added at same temperature. Then the resulting reaction mixture was stirred at 45° C. and added tBu Nitrite then the reaction was stirred at 45° C. for 3 h. The reaction was monitored by TLC (50% ethyl acetate in hexane as mobile phase) which confirmed that the reaction was completed after 3 h of stirring at 45° C. The resulting reaction mixture was cooled at RT then the reaction mixture was diluted with ethyl acetate (100 mL) and wash with solution of sodium sulphite (100) and NaCl solution then organic dried over Na₂SO₄ and concentrated under reduce pressure to obtain crude material which was purified by column chromatography using hexane and ethyl acetate. Product was eluted at 10% ethyl acetate in hexane to afford 5-iodo-2-methyl-2H-tetrazole (A27) (0.145 g, 0.690 mmol, Yield: 68.72%).

¹H NMR (DMSO-d₆, 400 MHz): δ ppm, 4.39 (s, 3H)

LCMS (Method A): 1.178 min, 100%, 254.0 nm, MS: ES+ 211.05 (M+1)

### Step-1:

To a stirred solution of A9 (0.05 g, 0.108 mmol, 1.0 eq), and A27 (0.023 g, 0.108 mmol, 1.0 eq) in dioxane:water (1.0:0.3 mL) in a 10 mL glass vial at room temperature was added Na₂CO₃ (0.035 g, 0.325 mmol, 3.0 eq). The resulting reaction mixture was stirred at RT under nitrogen purging for 15 min. PdCl₂(dppf) (0.0038 g, 0.050 mmol, 0.05 eq) was added at RT and the vial was then sealed and heated to 110° C. with stirring maintained for 2 h. The reaction was monitored by TLC (100% ethyl acetate in hexane as mobile phase) which confirmed that the reaction was completed after 2 h of stirring at 110° C. The resulting reaction mixture was cooled at RT then poured in cold water (50 mL) and extracted with ethyl acetate (30×2) organic layer was dried over Na₂SO₄ and concentrated under reduce pressure to obtain crude material which was purified by column chromatography using hexane and ethyl acetate. Product was eluted at 70% ethyl acetate in hexane and further purified by Prep HPLC purification Method-A to afford 3-methyl-3-(3-(2-methyl-2H-tetrazol-5-yl)-4-((4-(trifluoromethyl) phenyl) amino) phenyl) pyrrolidin-2-one (Compound 11) (0.031 g, 0.0744 mmol, Yield: 34.44%).

¹H NMR (DMSO-d₆, 400 MHz): δ ppm, 8.72 (s, 1H), 8.02 (s, 1H), 7.89 (s, 1H), 7.55-7.52 (m, 4H), 7.20 (d, J=8.4 Hz, 2H), 4.44 (s, 3H), 3.30-3.24 (m, 1H), 3.18-3.12 (m, 1H), 2.45-2.38 (m, 1H), 2.22-2.17 (m, 1H), 1.43 (s, 3H).

LCMS (Method A): 2.439 min, 100%, 254.0 nm, MS: ES+ 417.3 (M+1)

HPLC (Method B): 8.506 min, 99.48%, 210.0 nm

### Example 10—Synthesis of 3-methyl-3-(3-(pyridazin-3-yl)-4-((4-(trifluoromethyl) phenyl) amino) phenyl) pyrrolidin-2-one (Compound 12)

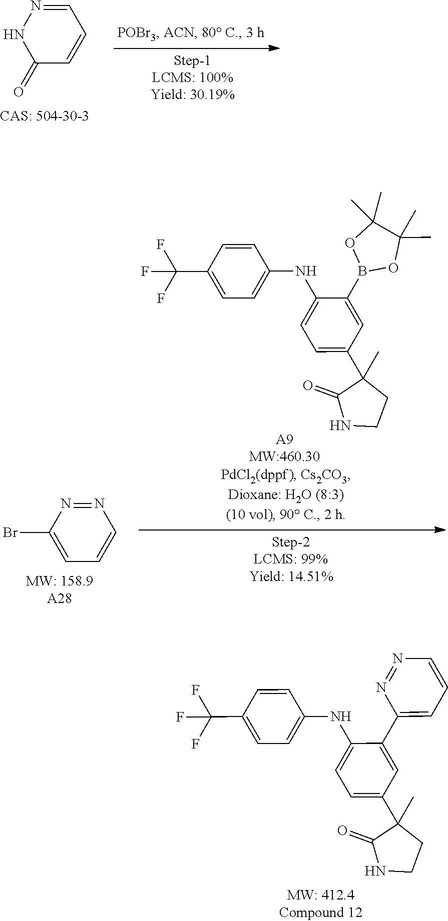 [SMILES: O=c1cccn[nH]1 ; CC1(c2ccc(Nc3ccc(C(F)(F)F)cc3)c(B3OC(C)(C)C(C)(C)O3)c2)CCNC1=O ; Brc1cccnn1 ; CC1(c2ccc(Nc3ccc(C(F)(F)F)cc3)c(-c3cccnn3)c2)CCNC1=O]

### Step-1:

To a solution of pyridazin-3(2H)-one (CAS 504-30-3) (0.15, 0.40 mmol, 1.0 eq) in ACN (3 mL) at RT, was added POBr₃ (0.032 g, 0.80 mmol, 2.0 eq) and the resulting mixture was heated to 80° C. and stirring was maintained for 3 h. The reaction was monitored on TLC (using EtOAc:Hex; 1:1 as mobile phase) which confirmed that the reaction was completed after 3 h. The resulting reaction mixture was quenched with sat. NaHCO₃ (5 mL) and extracted with EtOAc (3×20 mL). The combined organic layers were dried over Na₂SO₄ and concentrated under reduced pressure to afford 0.35 g crude. The obtained crude material was purified by flash column chromatography using silica (230-400 mesh), desired product eluted 40% EA in Hexane) Yielding 3-bromopyridazine (A28) (0.15 g, 0.943 mmol, Yield: 30.19%).

¹H NMR (DMSO-d6, 400 MHz): δ ppm, 9.27 (dd, J=4.8 Hz, 1.2 Hz 1H), 8.06 (dd, J=8.4 Hz, 1.2 Hz, 1H), 7.72-7.69 (m, 1H).

LCMS (Method A): 0.856 min, 100%, 254.0 nm, MS: ES+ 160.9 (M+2)

### Step-2:

To a solution of 3-methyl-3-(3-(4,4,5,5-tetramethyl-1,3,2-dioxaborolan-2-yl)-4-((4-(trifluoromethyl) phenyl) amino) phenyl) pyrrolidin-2-one (A9) (0.1 g, 0.217 mmol, 1.0 eq), 3-bromopyridazine (A28) (0.051 g, 0.329 mmol, 1.5 eq) in dioxane:H₂O (1 mL) was added caesium carbonate (0.211 g, 0.651 mmol, 3.0 eq), and the resulting mixture purged with N₂ for 10 min, after which was added PdCl₂(dppf) (0.0078 g, 0.010 mmol, 0.05 eq). The reaction mixture was heated at 90° C. with stirring for 2 h. The reaction was monitored on TLC (using DCM:MeOH; 9:1 as mobile phase). The resulting reaction mixture was quenched with ice-cold water (5 mL) and extracted with EtOAc (3×5 mL). The combined organic layers were dried over Na₂SO₄ and concentrated under reduced pressure to afford 0.15 g crude. The obtained crude material was purified by manual Flash chromatography using silica (200-400 mesh) as stationary phase (7% DCM in MeOH) yielding 3-methyl-3-(3-(pyridazin-3-yl)-4-((4-(trifluoromethyl) phenyl) amino) phenyl) pyrrolidin-2-one (Compound 12) (0.013 g, 0.0315 mmol, Yield: 14.51%).

¹H NMR (DMSO-d6, 400 MHz): δ ppm 9.17-9.16 (d, J=4.8 Hz, 1H), 8.93 (s, 1H), 7.88-7.86 (m, 2H), 7.73-7.68 (m, 2H), 7.55-7.53 (m, 1H), 7.46-7.41 (m, 3H), 6.93 (d, J=8.4 Hz, 2H), 3.30-3.26 (m, 1H), 3.20-3.17 (m, 1H), 2.23-2.18 (m, 1H), 1.45 (s, 3H); 1H merged with MeOD solvent peak which is clearly visible in DMSO NMR

¹H NMR (MeOD, 400 MHz): δ ppm, 9.11-9.10 (m, 1H), 8.07-8.05 (m, 1H), 7.78-7.72 (m, 2H), 7.59-7.52 (m, 2H), 7.41 (d, J=8.4 Hz, 2H), 7.03 (d, J=8.4 Hz, 2H), 2.64-2.58 (m, 1H), 2.38-2.31 (m, 1H), 1.61 (s, 3H), 1.21 (s, 1H).

LCMS (Method A): 2.175 min, 99%, 254.0 nm, MS: ES+ 412 (M+1)

HPLC (Method A): 7.58 min, 99.24%, 254.0 nm

Chiral HPLC (Method A): 4.31 min, 51.14%, 240.0 nm; 4.80 min, 48.85%, 240.0 nm

### Example 11—Synthesis of 3-methyl-3-(3-(pyrazin-2-yl)-4-((4-(trifluoromethyl) phenyl) amino) phenyl) pyrrolidin-2-one (Compound 13)

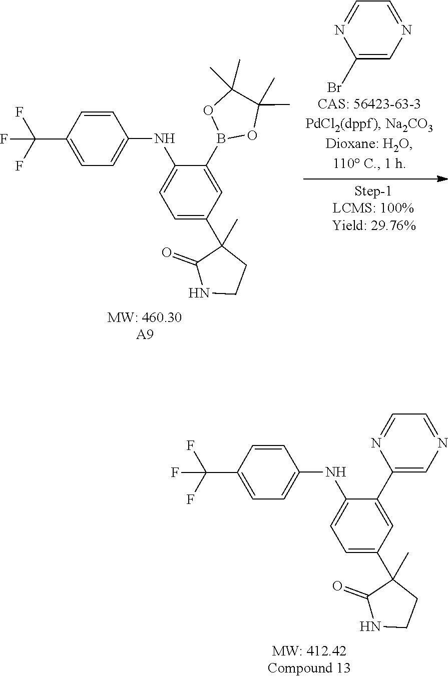 [SMILES: CC1(c2ccc(Nc3ccc(C(F)(F)F)cc3)c(B3OC(C)(C)C(C)(C)O3)c2)CCNC1=O ; Brc1cnccn1 ; CC1(c2ccc(Nc3ccc(C(F)(F)F)cc3)c(-c3cnccn3)c2)CCNC1=O]

### Step-1:

A stirred solution of (A9) (0.15 g, 0.325 mmol, 1.0 eq) in dioxane:H₂O (7:3) was prepared in 10 ml glass vial at room temperature. To this reaction solution, (CAS: 56423-63-3 (0.05 g, 0.314 mmol, 1.0 eq), Na₂CO₃ (0.10 g, 1.02 mmol, 3.0 eq) was added at same temperature under N₂ atmosphere. After that PdCl₂(dppf) (0.01 g, 0.01 mmol, 0.05 eq) was added, stirred, heat at 110° C. for 1 h. The reaction was monitored by TLC (MeOH:DCM, 1:9) which confirmed that the reaction was completed after 1 h of stirring at 110° C. The resulting reaction mixture was extracted by EA (20 ml) H₂O (20 ml). Organic layer was dried on Na₂SO₄, solvent removed on rotary evaporator and purified by glass column chromatography using 230-400 mesh size silica. Product (Compound 13) eluted in 10% MeOH:DCM (0.040 g, 0.096 mmol Yield: 29.76%)

¹H NMR (DMSO-d₆, 400 MHz): δ ppm, 8.85 (d, J=11.6 Hz, 2H), 8.72 (s, 1H), 8.55 (d, J=2.4 Hz, 1H), 7.86 (s, 1H), 7.72 (d, J=1.6 Hz, 1H), 7.54-7.51 (m, 1H), 7.42 (d, J=8.4 Hz, 3H).), 6.93 (d, J=8.4 Hz, 2H), 3.27-3.24 (m, 1H), 3.20-3.16 (m, 1H), 2.22-2.15 (m, 1H), 1.44 (s, 3H), 1H merged with DMSO solvent peak which is clearly visible in MeOD NMR

¹H NMR (MeOD, 400 MHz): δ ppm, 8.97 (s, 1H), 8.69 (s, 1H), 8.50 (d, J=2.4 Hz, 1H), 7.79 (d, J=1.6 Hz, 1H), 7.56-7.49 (m, 2H), 7.42 (d, J=8.4 Hz, 2H), 7.03 (d, J=8.4 Hz, 2H), 3.44-3.38 (m, 1H), 2.62-2.58 (m, 1H), 2.38-2.33 (m, 1H), 1.61 (s, 3H)

LCMS (Method A): 2.30 min, 100%, 254.0 nm, MS: ES+ 413.2 (M+1)

HPLC (Method B): 8.19 min, 100%, 254 nm.

### Example 12—Chiral Separation of 5-methyl-5-(3-(1-methyl-1H-imidazol-4-yl)-4-((4-(trifluoromethyl) phenyl)amino)phenyl)imidazolidine-2,4-dione (Compounds 14 and 15)

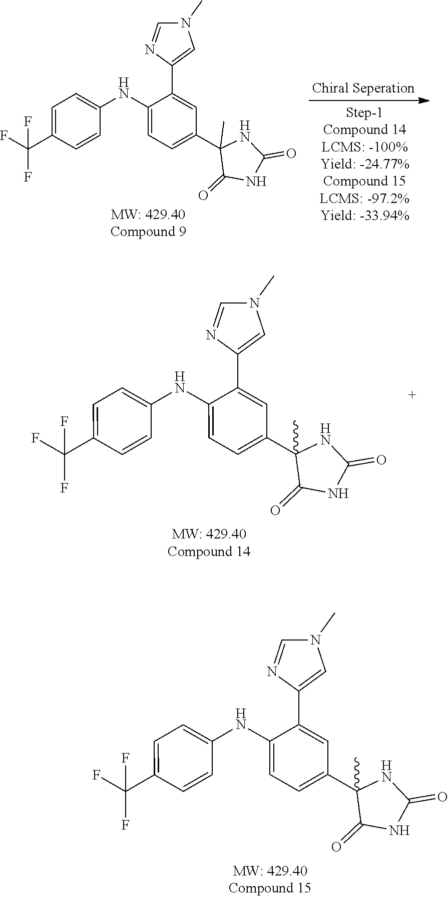

### Procedure:

Compound 9 (0.109 g) racemic was subjected to chiral SFC purification on (CHIRALPAK IA 250×4.6 mm 5 μm) column where 2 peaks were separated Compound 9

Peak-1 (Compound 14) (0.027 g, Yield=24.77%), and Compound 9 Peak-2 (Compound 15) (0.037 g, Yield=33.94%)

COLUMN ID: CHIRALPAK IA, 250*4.6 mm, 5 micron

MOBILE PHASE A: LIQ. CO2

MOBILE PHASE B: 0.1% M. NH3 IN MEOH-ACN (50-50)

FLOW RATE (ML/MIN): 4 mL

INSTRUMENT ID: Waters SFC Investigator system with 2998 PDA detector.

METHOD: Isocratic method of 70:30; with Flow rate=4 ml/min; Column oven temperature=40° C.; Back pressure=100 bar and analysis time 9 min.

Input Quantity: 0.109 g.

Output Quantity: Compound 14=0.027 g (% Yield=24.77%) and Compound 15=0.037 g (% Yield=33.94%)

### Compound 14

¹H NMR (DMSO-d₆, 400 MHz): δ ppm, 10.76 (brs, 1H), 9.92 (s, 1H), 8.60 (s, 1H), 7.83 (d, J=2.0 Hz, 1H), 7.79 (s, 1H), 7.58 (s, 1H), 7.51 (d, J=8.4 Hz, 2H), 7.41 (d, J=8.8 Hz, 1H), 7.28 (dd, J=8.4 Hz, 2.0 Hz, 1H), 7.11 (d, J=8.4 Hz, 2H), 3.71 (s, 3H), 1.68 (s, 3H).

¹H NMR (MeOD, 400 MHz): δ ppm), 7.81 (d, J=2.4 Hz, 1H), 7.70 (s, 1H), 7.47-7.44 (m, 3H), 7.41-7.39 (m, 2H), 7.09 (d, J=8.4 Hz, 2H), 3.76 (s, 3H), 1.82 (s, 3H).

LCMS (Method A): 1.70 min, 100.00%, 298.0 nm, MS: ES+ 430.3 (M+1)

HPLC (Method B): 5.24 min, 100%, 254.0 nm

CHIRAL HPLC: 3.25 min, 100.00%, 240.0 nm

### Compound 15

¹H NMR (DMSO-d₆, 400 MHz): δ ppm, 10.77 (s, 1H), 9.92 (s, 1H), 8.60 (s, 1H), 7.81 (dd, J=16.8 Hz, 2.4 Hz, 2H), 7.58 (s, 1H), 7.51 (d, J=8.8 Hz, 2H), 7.41 (d, J=8.4 Hz, 1H), 7.28 (dd, J=8.8 Hz, 2.4 Hz, 1H), 7.11 (d, J=8.4 Hz, 2H), 3.71 (s, 3H), 1.68 (s, 3H).

¹H NMR (MeOD, 400 MHz): δ ppm), 7.81 (d, J=2.4 Hz, 1H), 7.71 (s, 1H), 7.46-7.44 (m, 3H), 7.41-7.37 (m, 2H), 7.08 (d, J=8.4 Hz, 2H), 3.76 (s, 3H), 1.82 (s, 3H)

LCMS (Method A): 1.69 min, 97.02%, 254.0 nm, MS: ES+ 430.4 (M+1)

HPLC (Method B): 5.01 min, 96.44%, 210.0 nm

CHIRAL HPLC: 3.73 min, 93.63%, 240.0 nm

### Example 13—Synthesis of 3-methyl-3-(3-(1-methyl-1H-imidazol-4-yl)-4-((4-(trifluoromethyl) phenyl) amino) phenyl) pyrrolidine-2,4-dione (Compound 16)

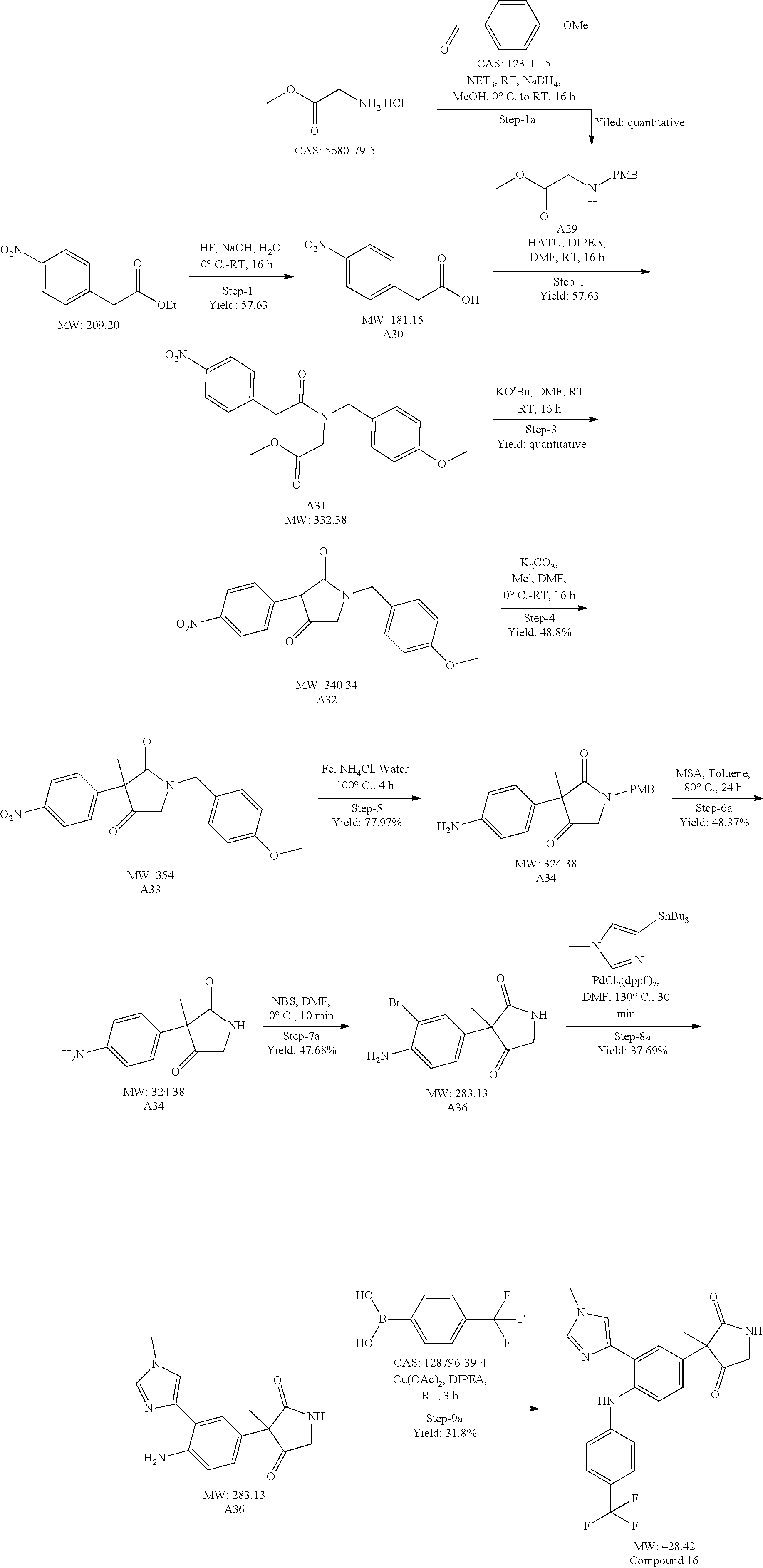

### Step-1a

A solution of methyl glycinate hydrochloride (CAS:5680-79-5) (275 g, 2190.3 mmol 1.0 eq) in methanol (100 mL) was cooled to 0° C., then TEA (332.4 g, 3285 mmol 1.5 eq) was added. The mixture was stirred for 30 min, then 4-methoxybenzaldehyde CAS:123-11-5 (298.2 g, 2190.3 mmol 1.0 eq) was added, and the mixture was stirred for 4 h at rt. The solution was cooled to 0° C. and then NaBH₄ (165.5 g, 4380.7 mmol 2.0 eq) was added portionwise to the reaction mixture. After stirring for 16 h at rt, the reaction was monitored on TLC (using EtOAc:Hex; 4:6 as mobile phase). The resulting reaction mixture was filtered, and the filtrate was distilled out and water (500 mL) was added. The mixture was extracted with EtOAc (4×500 mL). The combined organic layers were dried over Na₂SO₄ and concentrated under reduced pressure to afford 202 g crude. The obtained crude material was used directly for the next step without any further purification. The reaction yielded methyl (4-methoxybenzyl) glycinate (A29) (202 crude, 965.9 mmol, yield: quantitative)

¹H NMR (DMSO-d6, 400 MHz): δ ppm, 7.21 (d, J=8.8 Hz, 2H), 6.86 (dd, J=6.8 Hz, 2.0 Hz, 2H), 3.73 (s, 3H), 3.62 (br s, 2H), 3.61 (s, 3H) 3.28 (s, 2H) LCMS (Method A): 0.951 min, 81.11%, 220.0 nm, MS: ES+ 210 (M+1)

### Step-1:

To a solution of ethyl 2-(4-nitrophenyl)acetate (400 g, 1912.04 mmol, 1.0 eq) in THE was added NaOH solution (229 g, 5736 mmol, 3.0 eq) in H₂O (800 mL) at 0° C., then the reaction mixture was stirred at RT for 16 h. The reaction was monitored on TLC (using MeOH:DCM 1:9 as mobile phase). The resulting reaction mixture was distilled out and then water (100 mL) was added. The mixture was acidified with 1N HCl and extracted with EtOAc (3×500 mL). The combined organic layers were dried over Na₂SO₄ and concentrated under reduced pressure to afford crude. The obtained crude material was purified by manual column using silica (100-200 mesh) as stationary phase (eluent 7% MeOH:MDC) to yield 2-(4-nitrophenyl) acetic acid (A30) (200 g, 1104.0 mmol, yield: 57.63%).

¹H NMR (DMSO-d6, 400 MHz): δ ppm, 12.59 (s, 1H), 8.193 (d, J=8.8 Hz, 2H), 7.56 (d, J 8.8 Hz, 2H), 3.79 (s, 2H).

### Step-2:

To a solution of 2-(4-nitrophenyl)acetic acid (A30) (144 g, 794.92 mmol, 1.0 eq) in DMF was added methyl (4-methoxybenzyl)glycinate (A29) (216.0 g, 1033 mmol, 1.3 eq) and DIPEA (112.7 g, 874 mmol, 1.1 eq), and the reaction mixture was stirred at RT followed by addition of HATU (453.3 g, 1192.3 mmol 1.5 eq), and the mixture was stirred for 16 h. The reaction was monitored on TLC (using EtOAc:Hex; 5:5 as mobile phase). The resulting reaction mixture was poured in ice cold water (1000 mL) resulting in brown solid precipitate, and the mixture was stirred for 30 min then filtered and dried. Crude material was directly used for the next step. The reaction yielded methyl N-(4-methoxybenzyl)-N-(2-(4-nitrophenyl) acetyl) glycinate (A31) (100 g crude, 268.5 mmol, yield: 33.78%).

¹H NMR (DMSO-d6, 400 MHz): δ ppm, 8.20-8.16 (m, 2H), 7.51-7.49 (m, 2H), 7.22-7.17 (m, 2H), 6.93-6.86 (m, 2H), 4.64 (s, 1H), 4.45 (s, 1H), 4.25 (s, 1H), 4.00 (s, 2H) 3.87 (s, 1H), 3.74 (s, 3H) 3.61 (s, 3H),

LCMS (Method A):2.139 min, 97.36%, 254.0 nm, MS: ES+ 373.2 (M+1)

### Step-3:

To a solution of N-(4-methoxybenzyl)-N-(2-(4-nitrophenyl) acetyl) glycinate (A31) (70.0 g, 187.98 mmol, 1.0 eq) in DMF (700 ml) was added Ko^{t}Bu (22.14 g, 197.37 mmol, 1.05 eq) portionwise at RT, and then the reaction mixture was stirred at RT for 16 h. The reaction was monitored on TLC (using MeOH:DCM; 5:5 as mobile phase) which confirmed that the reaction was complete after 16 h. The resulting reaction mixture was quenched with water (1000 mL) and then acidified with dilute HCl, resulting in yellow solid precipitate. The mixture was stirred for 30 min and then filtered and dried to afford 1-(4-methoxybenzyl)-3-(4-nitrophenyl) pyrrolidine-2,4-dione (A32) (75 g, 220.37 mmol, yield: quantitative).

¹H NMR (DMSO-d6, 400 MHz): δ ppm, 12.71 (br s, 1H), 8.40-8.37 (m, 2H), 8.24-8.20 (m, 2H), 7.19 (d, J=8.8 Hz. 2H), 6.93 (d, J=8.8 Hz, 2H), 4.50 (s, 2H), 3.89 (s, 2H), 3.73 (s, 3H), LCMS (Method A): 2.092 min, 99.16%, 230.0 nm, MS: ES+ 341.2 (M+1)

### Step-4:

A solution of 1-(4-methoxybenzyl)-3-(4-nitrophenyl) pyrrolidine-2,4-dione (A32) (65.0 g, 190.99 mmol, 1.0 eq) in DMF (650 mL) was cooled to 0° C., then K₂CO₃ (28.9 g, 210.09 mmol, 1.1 eq) was added. The resulting mixture was stirred for 15 min, then Mel (40.3 g, 286.48 mmol, 1.5 eq) was added dropwise at 0° C. The resulting mixture was stirred at RT for 16 h. The reaction was monitored on TLC (using EtOAc:Hex; 4:6 as mobile phase) which confirmed that the reaction was completed after 16 h. The resulting reaction mixture was quenched with ice cold water (200 mL) and extracted with EtOAc (4×500 mL). The organic layer was washed with cold brine solution. The combined organic layers were dried over Na₂SO₄ and concentrated under reduced pressure to afford 53 g crude. The obtained crude material was purified by column chromatography silica (100-200 mesh) as stationary phase (24% Ethyl acetate in hexane) to afford 1-(4-methoxybenzyl)-3-methyl-3-(4-nitrophenyl) pyrrolidine-2,4-dione (A33) (33 g, 93.22 mmol, Yield: 48.80%).

¹H NMR (DMSO-d6, 400 MHz): δ ppm, 8.26-8.22 (m, 2H), 7.64-7.60 (m, 2H), 7.25 (d, J=8.4 Hz, 2H), 6.95-6.92 (m, 2H), 4.65 (d, J=14.8 Hz, 1H), 4.55 (d, J=14.8 Hz 1H), 4.03 (s, 2H), 3.74 (s, 3H), 3.61 (s, 3H),

LCMS (Method A): 2.336 min, 99.24%, 254.0 nm, MS: ES+ 355 (M+1)

### Step-5:

To a stirred solution of 1-(4-methoxybenzyl)-3-methyl-3-(4-nitrophenyl) pyrrolidine-2,4-dione (A33) (28.0 g, 79.09 mmol, 1.0 eq) in water (280 mL) at RT was added NH₄Cl (41.9 g, 790.9 mmol, 10.0 eq) and Fe (22.06 g, 395.48 mmol, 5.0 eq). The reaction mixture was heated to 100° C., then stirred for 5 h. The reaction was monitored by TLC (using: MeOH:DCM 1:9 as mobile phase) which confirmed that the reaction was complete after 5 h. The resulting reaction mixture was filtered then washed with 10% MeOH in DCM (400 ml), then the aqueous layer was extracted with 10% MeOH in DCM (400 ml). The combined organic layers were dried over Na₂SO₄ and concentrated under reduced pressure to afford 24 g crude. The obtained crude material was purified by trituration methanol/ether to afford 3-(4-aminophenyl)-1-(4-methoxybenzyl)-3-methyl pyrrolidine-2,4-dione (A34) (20 g, 61.65 mmol, Yield: 77.97%).

¹H NMR (DMSO-d6, 400 MHz): δ ppm, 7.23 (d, J=8.4 Hz, 2H), 6.92 (d, J=8.4 Hz, 2H), 6.90 (d, J=8.4 Hz, 2H), 6.50 (d, J=8.8 Hz, 2H), 5.16 (s, 2H), 4.69 (d, J=14.8 Hz, 1H), 4.43 (d, J 14.8 Hz, 2H), 3.92-3.74 (m, 2H), 3.72 (s, 3H), 1.39 (s, 3H)

LCMS (Method A): 1.684 min, 100%, 254.0 nm, MS: ES+ 325 (M+1)

### Step-6a:

To a stirred solution of 3-(4-aminophenyl)-1-(4-methoxybenzyl)-3-methyl pyrrolidine-2,4-dione (A34) (23.0 g, 70.90 mmol, 1.0 eq) in toluene was added MSA (CAS:75-75-2) (55.2 ml, 2.4 v) at rt, and then the reaction was stirred at 80° C. The reaction was monitored by TLC (using DCM:MeOH; 9:1 as mobile phase) which confirmed that the reaction was complete after 24 h of stirring at 80° C. The resulting reaction mixture was stirred and then settled. Toluene was decanted and then water (200 ml) was added, and the resulting mixture was washed with ethyl acetate (4×300 mL). The aqueous layer was basified with NaHCO₃ then extracted with ethyl acetate (4×300 mL). The combined organic layers were dried over Na₂SO₄ and concentrated under reduced pressure to afford 3-(4-aminophenyl)-3-methylpyrrolidine-2,4-dione (A35) (7.0 g, 34.29 mmol, yield: 48.37%).

¹H NMR (DMSO-d6, 400 MHz): δ ppm, 8.55 (s, 1H), 6.91 (d, J=8.8 Hz, 2H), 6.52 (d, J=8.8 Hz, 2H), 5.16 (s, 2H), 4.0-3.90 (m, 2H), 1.34 (s, 3H),

LCMS (Method A): 0.368 min, 100%, 210.0 nm, MS: ES+ 205 (M+1)

### Step-7a:

A stirred solution of 3-(4-aminophenyl)-3-methylpyrrolidine-2,4-dione (A35) (4.0 g, 19.59 mmol, 1.0 eq) in DMF was cooled to 0° C. NBS (2.44 g, 13.61 mmol, 0.7 eq) was added portionwise, and the resulting mixture was stirred for 10 min. The reaction was monitored by TLC (using DCM:MeOH; 7:3 as mobile phase) which confirmed that the reaction was complete. The reaction mixture was diluted with ice cold water (100 mL) and extracted with EtOAc (3×250 mL). The organic layer was separated and washed with ice cold water and brine, then dried over Na₂SO₄ and concentrated under reduced pressure to afford 3.2 g crude. The obtained crude material was purified by column chromatography using silica (100-200 mess) as stationary phase (1.4% DCM in MeOH) to afford 3-(4-amino-3-bromophenyl)-3-methylpyrrolidine-2,4-dione (A36) (2.57 g, 9.11 mmol, Yield:47.68%).

¹H NMR (DMSO-d6, 400 MHz): δ ppm, 8.63 (s, 1H), 7.20 (d, J=2 Hz, 1H), 6.99-6.97 (d, d, J=2 Hz, J=2 Hz, 1H), 6.75 (d, J=8.4 Hz 1H), 4.09-3.90 (m, 2H), 1.36 (s, 3H)

LCMS (Method A): 1.491 min, 100%, 254.0 nm, MS: ES+ 283 (M+1)

### Step-8a:

To a stirred solution of 3-(4-amino-3-bromophenyl)-3-methylpyrrolidine-2,4-dione (A36) (4.5 g, 15.8 mmol, 1.0 eq) in DMF (15 ml) was added 1-methyl-4-(tributylstannyl)-1H-imidazole (11.7 g, 31.78 mmol, 2.0 eq). The resulting mixture was stirred at RT and purged with N₂(g) for 15 min, followed by addition of PdCl₂(dppf)₂ (2.324 g, 3.18 mmol, 0.2 eq). The resulting mixture was stirred at RT and purged with N₂(g) for 15 min, then reaction was heated in microwave to 130° C. for 30 min. The reaction was monitored by TLC (using DCM:MeOH; 9:1 as mobile phase) which confirmed that the reaction was complete after 30 min. The reaction mixture was extracted with EtOAc (2×200 mL). The organic layer was separated and washed with brine (200 ml), then dried over Na₂SO₄ and concentrated under reduced pressure to afford 2.0 g crude. The obtained crude material was purified by column chromatography silica (100-200 mess) as stationary phase (2% MeOH in DCM) to afford 3-(4-amino-3-(1-methyl-1H-imidazol-4-yl) phenyl)-3-methylpyrrolidine-2,4-dione (A37) (1.7 g, 5.97 mmol, yield: 37.69%)

¹H NMR (DMSO-d6, 400 MHz): δ ppm, 8.56 (s, 1H), 7.69 (s, 1H), 7.57-7.45 (m, 3H), 7.23 (d, J=2.4 Hz, 1H), 6.83-6.80 (m, 1H), 6.62 (d, J=8.4 Hz, 1H), 6.23 (s, 2H), 4.09-3.98 (m, 2H), 3.71 (s, 3H), 1.39 (s, 3H),

LCMS (Method A): 0.940 min, 80.71%, 254.0 nm, MS: ES+ 285.1 (M+1)

### Step-9a:

To a stirred solution of 3-(4-amino-3-(1-methyl-1H-imidazol-4-yl)phenyl)-3-methylpyrrolidine-2,4-dione (A37) (1.2 g, 4.22 mmol, 1.0 eq) in DCM (12 ml) was added (4-(trifluoromethyl)phenyl)boronic acid (CAS:128796-39-4) (0.80 g, 4.22 mmol, 1.0 eq), DIPEA (1.6 g, 12.6 mmol, 3.0 eq), and Cu(OAC)₂ (1.14 g, 6.33 mmol, 31.5 eq) at RT. The resulting mixture was stirred at RT in open atmosphere for 3 h. The reaction was monitored by TLC (using DCM:MeOH; 9:1 as mobile phase) which confirmed that the reaction was complete after 3 h. The reaction mixture was quenched with water (200 ml) and extracted with DCM (3×100 mL). The organic layer was dried over Na₂SO₄ and concentrated under reduced pressure to afford 0.850 g crude. The obtained crude material was purified by column chromatography using silica (230-400 mesh) as stationary phase (10% MeOH in DCM) to afford 3-methyl-3-(3-(1-methyl-1H-imidazol-4-yl)-4-((4-(trifluoromethyl) phenyl) amino) phenyl) pyrrolidine-2,4-dione (Compound 16) (0.574 g, 1.33 mmol, yield: 31.8%).

¹H NMR (DMSO-d6, 400 MHz): δ ppm, 9.92 (s, 1H), 8.68 (s, 1H), 7.78 (s, 1H), 7.64 (d, J=2.0 Hz, 1H), 7.57 (d, J=1.2 Hz, 1H), 7.50 (d, J=8.4 Hz, 2H), 7.38 (d, J=8.4 Hz, 1H), 7.11 (d, J 8.8 Hz, 3H), 4.09-3.98 (m, 2H), 3.70 (s, 3H), 1.49 (s, 3H),

LCMS (Method A): 1.851 min, 97.00%, 254.0 nm, MS: ES+ 429.2 (M+1)

HPLC (Method A): 7.71 min, 98.83%, 254.0 nm

Chiral HPLC: 9.118 min, 44.84%, 320.0 nm; 9.607 min, 55.159%, 320.0 nm

### Example 14—Chiral separation of 3-methyl-3-(3-(1-methyl-1H-imidazol-4-yl)-4-((4-(trifluoromethyl) phenyl) amino) phenyl) pyrrolidine-2,4-dione (Compound 17 and Compound 18)

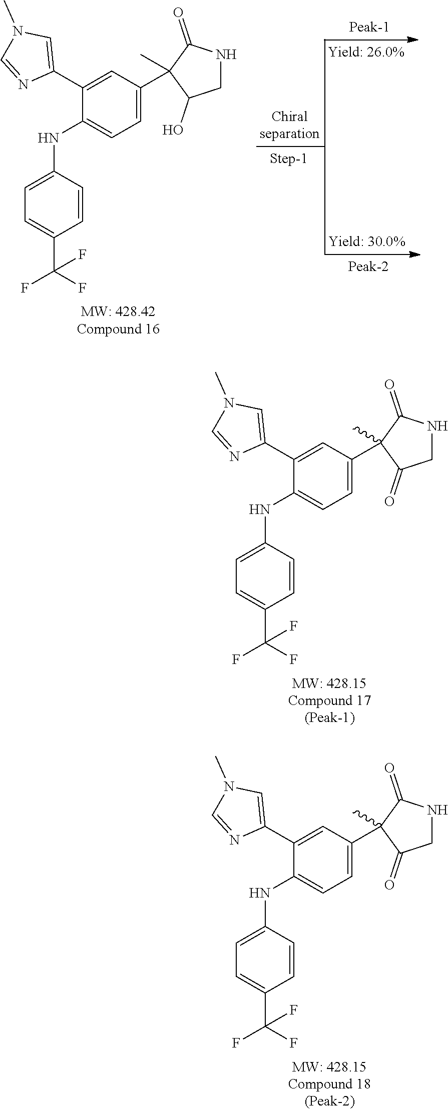

### Chiral Separation of Compound 17 & Compound 18:

Compound 16 (0.1 g) racemic was subjected to chiral SFC purification on YMC CELLULOSE SC (250×20 mm 5 μm) column where 2 peaks were separated Compound 17 and Compound 18.

COLUMN ID: YMC CELLULOSE SC (250×20 mm 5 um)

MOBILE PHASE A: Liq.CO₂

MOBILE PHASE B: M.NH3-HEP-IPA (70-30)

FLOW RATE (ML/MIN): 24

INSTRUMENT ID: PHP-04-AGEILENT 1260 SERISE INFINITY-2 WITH UV DETECTOR

METHOD: TIME: FLOW: % A: % B (0:24:0:100), (32:24:0:100)

Input Quantity: 0.1 g.

Output Quantity: Compound 17=0.026 g (% Yield=26.0%) and Compound 18=0.030 g (% Yield=30.0%)

### Compound 17:

¹H NMR (DMSO-d6, 400 MHz): δ ppm, 9.91 (s, 1H), 8.69 (s, 1H), 7.79 (s, 1H), 7.62 (d, J=2.0 Hz, 1H), 7.57 (s, 1H), 7.50 (d, J=8.8 Hz, 2H), 7.38 (d, J=8.4 Hz, 1H), 7.10 (d, J=8.4 Hz, 3H), 4.09-3.98 (m, 2H), 3.70 (s, 3H), 1.49 (s, 3H)

LCMS (Method A): 1.797 min, 99.91%, 254.0 nm, MS: ES+ 429.3 (M+1)

HPLC (Method A): 5.508 min, 99.88%, 254.0 nm

Chiral HPLC (Method A): 14.99 min, 100%, 262 nm

### Compound 18:

¹H NMR (DMSO-d6, 400 MHz): δ ppm, 9.92 (s, 1H), 8.69 (s, 1H), 7.78 (s, 1H), 7.62 (d, J=2.0 Hz, 1H), 7.57 (d, J=1.2 Hz, 1H), 7.50 (d, J=8.8 Hz, 2H), 7.38 (d, J=8.8 Hz, 1H), 7.11 (d, J=8.4 Hz, 3H), 4.09-3.98 (m, 2H), 3.70 (s, 3H), 1.49 (s, 3H)

LCMS (Method A): 1.792 min, 100%, 254.0 nm, MS: ES+ 429.3 (M+1)

HPLC (Method A): 5.497 min, 100%, 254.0 nm

Chiral HPLC (Method A): 16.90 min, 98.02%, 262 nm

### Example 15—Synthesis of 4-hydroxy-3-methyl-3-(3-(1-methyl-1H-imidazol-4-yl)-4-((4-(trifluoromethyl) phenyl) amino) phenyl) pyrrolidin-2-one (Compound 19)

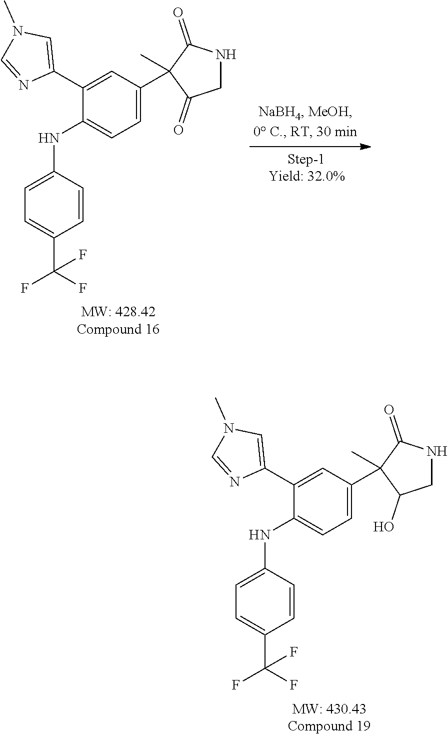 [SMILES: Cn1cnc(-c2cc(C3(C)C(=O)CNC3=O)ccc2Nc2ccc(C(F)(F)F)cc2)c1 ; Cn1cnc(-c2cc(C3(C)C(=O)NCC3O)ccc2Nc2ccc(C(F)(F)F)cc2)c1]

To a stirred solution 3-methyl-3-(3-(1-methyl-1H-imidazol-4-yl)-4-((4-(trifluoromethyl) phenyl) amino) phenyl) pyrrolidine-2,4-dione (Compound 16) (0.100 g, 0.233 mmol, 1.0 eq) in MeOH (1.0 ml) and DCM (1.0 ml) was added NaBH₄ at 0° C. (0.013 g, 0.350 mmol, 1.5 eq), and the resulting mixture was stirred at RT for 30 min. The reaction was monitored by TLC (using DCM:MeOH; 9:1 as mobile phase) which confirmed that the reaction was complete after 30 min of stirring at RT. The reaction mixture was diluted with CHCl₃, and then NH₄Cl solution in water (5 mL) was added. The mixture was extracted with DCM (3×20 mL), and organic layer was dried over Na₂SO₄ and concentrated under reduced pressure to afford 0.06 g crude. The obtained crude material was purified by reverse phase column chromatography as stationary phase (C₁₈), mobile phase (100% Water: ACN) to afford 4-hydroxy-3-methyl-3-(3-(1-methyl-1H-imidazol-4-yl)-4-((4-(trifluoromethyl)phenyl)amino)phenyl)pyrrolidin-2-one (Compound 19) (0.032 g, 0.0743 mmol, yield: 32%).

¹H NMR (DMSO-d6, 400 MHz): δ ppm, 9.76 (s, 1H), 7.88 (s, 1H), 7.76 (s, 1H), (s, 1H), 7.69 (s, 1H), 7.65 (d, J=2.0 Hz, 1H), 7.51 (d, J=10 Hz, 1H), 7.44 (s, 1H), 7.31 (d, J=8.4 Hz, 1H), 7.08 (d, J=8.4 Hz, 3H), 5.17 (s, 1H), 4.12 (t, J=5.8 Hz, 1H), 3.70 (s, 3H), 2.84 (s, 1H), 1.42 (s, 3H)

LCMS (Method A): 1.595 min, 100.0%, 254.0 nm, MS: ES+ 431 (M+1)

HPLC (Method A): 4.350 min, 100.0%, 254.0 nm

Chiral HPLC: (Peak-1) 6.42 min, 47.61%, 305.0 nm; (Peak-2) 6.76 min, 45.58%, 305.0 nm

### Example 16—Chiral separation of 3-methyl-3-(3-(2-methyl-2H-tetrazol-5-yl)-4-((4-(trifluoromethyl) phenyl) amino) phenyl) pyrrolidin-2-one (Compound 20 and Compound 21)

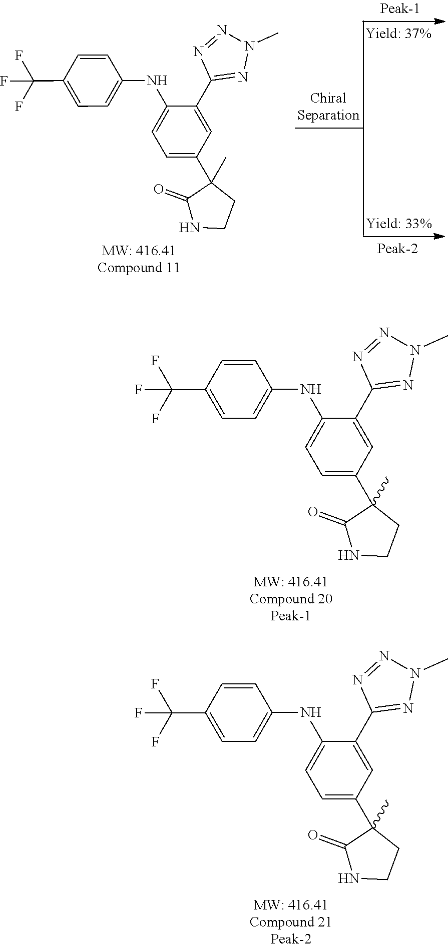

### Chiral Separation of Compound 20 and Compound 21:

Compound 11 (18.2 mg) racemic was subjected to chiral SFC purification on CHIRALPAK IG, 250×10 mm, 5 μm column where 2 peaks were separated Compound 20 and Compound 21.

COLUMN ID: CHIRALPAK IG, 250×10 mm, 5 μm

MOBILE PHASE A: 0.1% NH3 IN HEPTANE

MOBILE PHASE B: 0.1% NH3 IN IPA: MEOH (50:50)

FLOW RATE (ML/MIN): 6

INSTRUMENT ID: PHP-04-AGILENT 1260 SERIES INFINITY-II WITH UV DETECTOR

METHOD: TIME: FLOW: % A: % B (IITIAL: 38:6:6), (80:20)

Input Quantity: 18.2 mg.

Output Quantity: Compound 20=0.0677 g (% yield=37.2%) and Compound 21=0.061 g (% yield=33.52%)

### Compound 20:

¹H NMR (400 MHz, MeOD): δ ppm, 8.24 (d, J=2.4 Hz, 1H), 7.58-7.50 (m, 4H), 7.31 (d, J=8.4 Hz, 2H), 4.47 (s, 3H), 3.47-3.37 (m, 1H), 2.59-2.53 (m, 1H), 2.37-3.30 (m, 1H), 1.60 (s, 3H)

LCMS (Method A): 2.41 min, 100%, 254.0 nm, MS: ES+ 417 (M+1)

HPLC (Method A): 8.51 min, 100%, 254.0 nm

Chiral HPLC (Method A): 5.08 min, 100.0%, 304 nm

### Compound 21:

¹H NMR (400 MHz, MeOD): δ ppm, 8.24 (d, J=2.4 Hz, 1H), 7.58-7.50 (m, 4H), 7.31 (d, J 8.4 Hz, 2H), 4.47 (s, 3H), 3.47-3.44 (m, 1H), 3.43-3.37 (m, 1H), 2.59-2.53 (m, 1H), 2.37-2.30 (m, 1H), 1.60 (s, 3H

LCMS (Method A): 2.37 min, 98.96%,254.0 nm, MS: ES+ 417 (M+1)

HPLC (Method A): 8.52 min, 100%, 254.0 nm

Chiral HPLC (Method A): 5.33 min, 98.87%, 304 nm

### Example 17—Synthesis of 3-methyl-3-(3-(1-methyl-1H-imidazol-4-yl)-4-((3-(trifluoro methyl) phenyl) amino) phenyl) pyrrolidin-2-one (Compound 22)

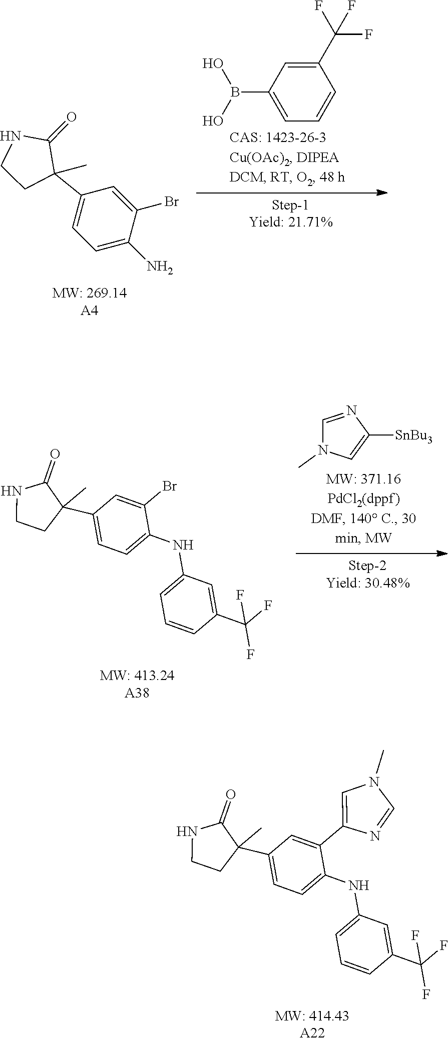

### Step-1:

To a solution 3-(4-amino-3-bromophenyl)-3-methylpyrrolidin-2-one (A4) (0.6 g, 2.230 mmol, 1.0 eq) in DCM (6 ml) were added CAS:1423-26-3 (0.632 g, 3.345 mmol, 1.5 eq), DIPEA (1.1 ml, 6.69 mmol, 3.0 eq), and Cu(OAc)₂(0.605 g, 3.345 mmol, 1.5 eq), and then the reaction mixture was stirred at RT for 48 h with vial open to air (i.e., without septum). The reaction was monitored on TLC (using 90% EtOAc in hexane as mobile phase). Water (10 ml) was added to the reaction mixture, and the mixture was extracted with DCM (3×25 mL). The combined organic layers were dried over Na₂SO₄ and concentrated under reduced pressure to afford 0.8 g crude. The obtained crude material was purified by manual column chromatography using silica (100-200 mesh) as stationary phase (desired product was eluted in 50-55% EtOAc in hexane as mobile phase) to afford 3-(3-bromo-4-((3-(trifluoromethyl) phenyl) amino) phenyl)-3-methylpyrrolidin-2-one (A38) (0.2 g, 0.484 mmol, yield: 21.71%).

¹H NMR (DMSO-d6, 400 MHz): δ ppm, 8.07 (s, 1H), 7.87 (b s, 1H), 7.66 (d, J=2.0 Hz, 1H), 7.42-7.29 (m, 3H), 7.20 (s, 1H), 7.15-7.0 (m, 2H), 3.17-3.13 (m, 2H), 2.41-2.13 (m, 2H), 1.38 (s, 3H).

LCMS (Method A): 2.352 min, 100%, 254.0 nm, MS: ES+ 413 (M+1)

### Step-2:

To a stirred solution of 3-(3-bromo-4-((3-(trifluoromethyl) phenyl) amino) phenyl)-3-methylpyrrolidin-2-one (A38) (0.18 g, 0.435 mmol, 1.0 eq) in DMF (2 mL) was added 1-methyl-4-(tributylstannyl)-1H-imidazole (0.323 g, 0.871 mmol, 2.0) eq) at rt, and the resulting mixture was purged with N₂(g) for 10 min at RT. Then PdCl₂(dppf) (0.063 g, 0.087 mmol, 0.2 eq) was added, and the mixture was stirred at 140° C. for 30 min in microwave. The reaction was monitored on TLC (using EA:HEX; 9.0:1.0 as mobile phase) which confirmed that the reaction was complete after 30 min of stirring at 140° C. The resulting reaction mixture was diluted with H₂O (20 mL) and extracted with EtOAc (3×25 mL). The combined organic layers were dried over Na₂SO₄ and concentrated under reduced pressure to afford 0.3 g crude. The obtained crude material was purified by flash column chromatography using silica (230-400 mesh) as stationary phase (75% EA in HEX) yielding 3-methyl-3-(3-(1-methyl-1H-imidazol-4-yl)-4-((4-(trifluoromethyl) phenyl) amino) phenyl) pyrrolidin-2-one (Compound 22) (0.055 g, 0.132 mmol, Yield: 30.48%)

¹H NMR (DMSO-d₆, 400 MHz): δ ppm, 9.78 (s, 1H), 7.79-7.72 (m, 3H), 7.58-7.57 (m, 1H), 7.39 (d, J=87.6 Hz, 1H), 7.30-7.20 (m, 4H), 7.08 (d, J=7.6 Hz, 1H), 3.72 (s, 3H), 3.36-3.15 (m, 2H), 2.46-2.13 (m, 2H), 1.42 (s, 3H).

LCMS (Method A): 1.67 min, 100%, 254.0 nm, MS: ES+ 415.17 (M+1).

HPLC (Method A): 4.68 min, 100%, 254.0 nm

CHIRAL HPLC (Method A): (Peak-1) 6.03 min, 50.52%, 300 nm; (Peak-2) 6.21 min, 49.47%, 300 nm

### Example 18—Synthesis of 1-(3-(1-methyl-1H-imidazol-4-yl)-4-((4-(trifluoromethyl) phenyl) amino) phenyl) ethan-1-one (Compound 23)

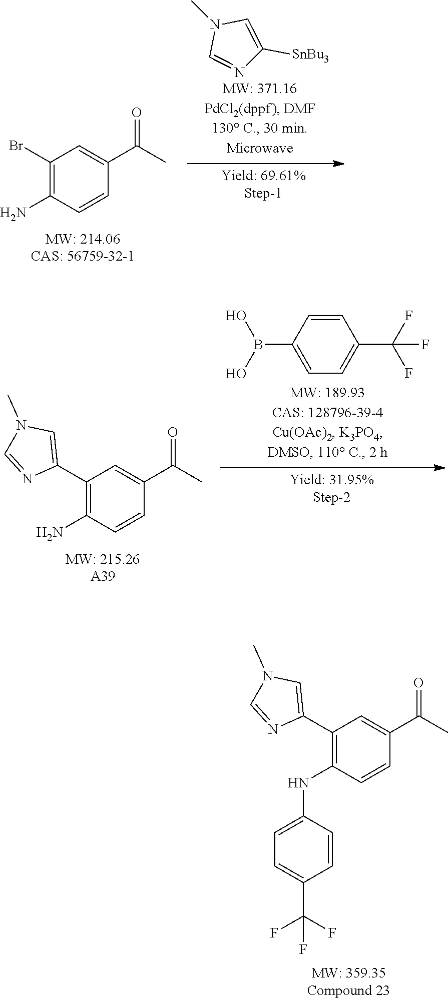

### Step-1:

A solution of 1-(4-amino-3-bromophenyl) ethan-1-one (CAS No: 56759-32-1) (0.5 g, 2.33 mmol 1.0 eq) in DMF 5 mL was prepared in 30 mL of glass microwave vial at room temperature. To this solution, 1-methyl-4-(tributylstannyl)-1H-imidazole (1.73 g, 4.672 mmol, 2.0 eq) was added at RT under nitrogen gas atmosphere. To the resulting reaction mixture was added PdCl₂(dppf) (0.17 g, 0.233 mmol, 0.1 eq), and the mixture was purged with N₂ gas. The reaction mixture was heated to 130° C. and stirred for 30 min. The reaction was monitored by TLC (70% EtOAc in hexane as a mobile phase) which confirmed that the reaction was complete after 30 min. at 130° C. The resulting reaction mixture was extracted with EA (3×100 mL). The combined organic layers were dried over Na₂SO₄, filtered and concentrated under reduced pressure to afford crude product (0.60 g). The obtained crude material was purified by Combi-Flash chromatography using silica gel (300-400 mesh) (desired product eluted in 50-55% EA in Hexane) to afford 1-(4-amino-3-(1-methyl-1H-imidazol-4-yl) phenyl) ethan-1-one (A39) (0.35 g, 1.627 mmol, yield: 69.61%).

¹H NMR (DMSO-d6, 400 MHz): δ ppm 8.00 (d, J=2.0 Hz 1H), 7.73 (s, 1H), 7.69 (s, 1H), 7.56 (dd, J=8.8 Hz, J=2.0 Hz, 1H), 7.22 (bs, 2H), 6.69 (d, J=8.4 Hz 1H), 3.72 (s, 3H), 2.44 (s, 3H)

LCMS (Method A): 1.01 min, 98.61%, 254 nm, MS: ES+ 216 (M+1)

### Step-2:

A stirred solution of 1-(4-amino-3-(1-methyl-1H-imidazol-4-yl) phenyl) ethan-1-one (A39) (3.5 g, 17.44 mmol 1.0 eq) in DMSO (37.5 mL) was prepared in 100 mL RBF at room temperature. To this solution was added (4-(trifluoromethyl) phenyl) boronic acid (CAS: 128796-39-4) (3.29 g, 17.44 mmol, 1.0 eq) and K₃PO₄ (7.39 g, 34.88 mmol, 2.0 eq) at RT under nitrogen gas atmosphere. To the resulting reaction mixture was added Cu(OAc)₂ (3.78 g, 20.92 mmol, 1.2 eq), and the mixture was purged with N₂ gas. The reaction mixture was heated to 110° C. and stirred for 2 h. The reaction was monitored by TLC (80% EtOAc in hexane as a mobile phase) which confirmed that the reaction was complete after 2 h at 110° C. The reaction mixture was extracted with EA (3×150 mL). The combined organic layers were dried over Na₂SO₄, filtered and concentrated under reduced pressure to afford crude product (5.5 g). The obtained crude material was purified by Combi-Flash chromatography using silica gel (300-400 mesh) (desired product eluted in 45-50% EA in Hexane) to afford 1-(3-(1-methyl-1H-imidazol-4-yl)-4-((4-(trifluoromethyl) phenyl) amino) phenyl) ethan-1-one (Compound 23) (4.0 g, 11.14 mmol, yield: 31.95%).

¹H NMR (DMSO-d6, 400 MHz): δ ppm, 11.15 (s, 1H), 8.23 (d, J=2.0 Hz 1H), 7.87 (s, 2H), 7.76 (dd, J=8.8 Hz, J=2.0 Hz, 1H), 7.64 (d, J=8.4 Hz 2H), 7.48 (d, J=8.4 Hz 1H), 7.37 (d, J 8.4 Hz 2H), 3.75 (s, 3H), 2.50 (s, 3H)

LCMS (Method A): 2.01 min, 99.01%, 254 nm, MS: ES+ 360 (M+1)

### BIOLOGICAL EXAMPLES

Prior art TEAD inhibitors 1-1, 1-2, 1-3, and 1-4 (structures provided in Table 3) are used as reference compounds throughout Biological Examples 19-29.

TABLE 3
Prior art TEAD inhibitors

| Compound Number | Structure |
|---|---|
| 1-1 | 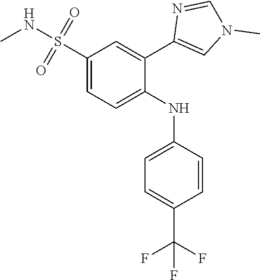 |
| 1-2 | 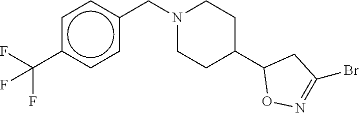 |
| 1-3 | 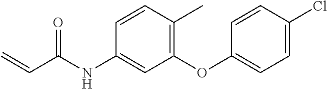 [SMILES: C=CC(=O)Nc1ccc(C)c(Oc2ccc(Cl)cc2)c1] |
| 1-4 | 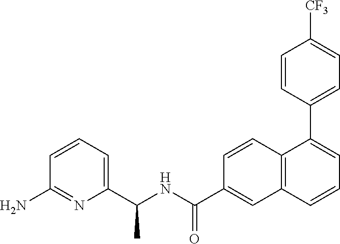 [SMILES: C[C@H](NC(=O)c1ccc2c(-c3ccc(C(F)(F)F)cc3)cccc2c1)c1cccc(N)n1] |

### Example 19—NCI-H226, H23, and H28 Proliferation and Cell Viability Assay

Cell proliferation of a TEAD dependent NF2-null mesothelioma cell line, NCI-H226 was assayed using two different protocols. Results were confirmed in two different TEAD-non-dependent lung cancer cell lines, H23 and H28, were used as non-responsive controls, as indicated:

a) CellTiter-Glo® 2.0 (Promega #G9243). The effect of the TEAD inhibitors disclosed herein on cell proliferation was determined in TEAD1-genetically dependent cell line NCI-H226 (responder cell line) and TEAD-independent cell lines (NCI-H23, HEK293T), as non-responder, negative control. Cells were seeded in 96-well plates (Thermo Scientific #167008) in growth media (RPMI-1640, Gibco #61870-010) for NCI-H226 and NCI-H23 or DMEM (Gibco #31966-021, HEK293T) with 100% Heat Inactivated Fetal Bovine Serum (Gibco 10500-064) at 800 (NCI-H226 and HEK-293T) or 2000 (NCI-H23) cells per well at 37° C., 500 CO₂, humidified incubator; 24 hours later, fresh growth media containing test compound or DMSO vehicle was added and incubated with the cells for 144 hrs. TEAD inhibitor stocks were made at a 1000× fold excess in DMSO, and a dose response test compound dose range of 10,000 to 0.1 nM was used. Each treatment was carried out in triplicate. After 144 hrs of treatment, relative cell viability numbers were determined using the CellTiter-Glo 2.0 assay following the manufacturer's instructions. Test plates and CellTiter-Glo 2.0 reagent were allowed to equilibrate to room temperature. CellTiter-Glo 2.0 reagent was added at 1:1 reagent to media ratio to the 96-well cell plates, plates were placed on a plate shaker for 2 minutes and then incubated at room temperature for a further 10 minutes to allow complete cell lysis to occur. The mix was then transferred to white, 96-well luminometer assay plates (Greiner Bio One #655075). Luminescence was quantified using a CLARIOStar Plus multi-mode plate reader (BMG Labtech) in luminescence detection mode with the recommended settings (ultra-Glo pre-set: emission 545-50 nm with autofocus and enhanced dynamic range on). Relative cell density was expressed as a function of inhibitor concentration, with respect to DMSO-only control wells. Data where plotted using GraphPad Prism and IC₅₀ values for the biological effect were calculated using the EC₅₀ non-linear fit function using a sigmoidal, 4-parameter logistic, least squares fit with no constraints.

b) Cell Titer One (Promega, #G3582). The effect of the TEAD inhibitors disclosed herein on cell proliferation was also determined using the Promega Substrate Cell Titer Aqueous One Solution Reagent. The TEAD inhibitors were evaluated for the effects on cell proliferation responder (H226) and non-responder negative control cancer cell lines (CAMA1, H28), as well as non-transformed normal human cells (HUVEC, NHDF). Dissociated cells were resuspend in media (depending on cell line, either in DMEM, RPMI, or EMEM all with 10% FBS, with gentamicin as an antibiotic). Cells were seeded at 100 μL/well at 750 cells per well in 96-well plates and allowed to settle and attach. After an incubation of 24 hrs (37° C., 5% CO₂, humidified incubator), 100 μL of test compound-containing media was added. A dilution series of 2000× concentrated stock of test compounds was prepared in DMSO, for final concentrations from 0.1 to 10,000 nM. After addition of test compound-containing media, cells were incubated for 6 days at 37° C., 5% CO₂, humidified incubators). To measure cell density, 20 μL of Promega Substrate Cell Titer 96 Aqueous One Solution Reagent (Promega, #G3582) to each well was added according to the manufacturer's instructions. Plates were incubated at 37° C. for usually 3-8 hrs, to reach in the control cells (without compound) an OD of 1.5 at 490 nm. The OD was measured using Vmax [Molecular Devices] spectrophotometer plate reader. Media only-wells were used as background controls and cell densities normalized to DMSO vehicle control. EC₅₀ were taken as the concentration achieving 50% of maximal growth inhibition.

For IC₅₀ and EC₅₀ data “A” indicates a value of less than 0.2 μM; the symbol “B” indicates a value in the range of from 0.2 μM up to 1 μM; and the symbol “C” indicates a value of greater than 1 μM. ND: no data.

TABLE 4
Cytotoxic potency measured by CTG assay for the
H226-responder and H23-non-responder cell lines

| | NCI-H226 | NCI-H23 |
|---|---|---|
| Cmpd No. | EC₅₀ | IC₅₀ |
| 1 | A | C |
| 2 | A | C |
| 3 | A | ND |
| 4 | A | ND |
| 5 | A | ND |
| 6 | A | ND |
| 7 | B | ND |
| 8 | A | ND |
| 9 | A | ND |
| 10 | A | ND |
| 11 | A | ND |
| 12 | A | ND |
| 13 | A | ND |
| 14 | A | ND |
| 15 | A | ND |
| 16 | A | ND |
| 17 | A | ND |
| 18 | A | ND |
| 19 | B | ND |
| 20 | A | ND |
| 21 | A | ND |
| 22 | A | ND |
| 23 | A | ND |
| 24 | A | ND |

TABLE 5
Cytotoxic potency measured by Cell Titer
One assay for the H226 and H28 cell lines

| | NCI-H226 | NCI-H28 |
|---|---|---|
| Cmpd No. | IC₅₀ | IC₅₀ |
| 1 | A | ND |
| 2 | A | ND |
| 3 | A | ND |
| 4 | A | ND |
| 5 | A | C |
| 6 | B | ND |
| 7 | A | ND |
| 8 | B | ND |
| 9 | A | ND |
| 10 | A | ND |
| 11 | A | ND |
| 12 | C | ND |
| 13 | C | ND |
| 14 | C | ND |
| 15 | A | ND |

### Example 20—Mero14 Proliferation and Cell Viability Assay

Mero14 cells were trypsinised, counted and plated at 400 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control is plotted against compound concentration and a curve fitted with non-linear regression in GraphPad Prism software. EC₅₀ and absolute IC₅₀ values were calculated using GraphPad Prism software as follows: For EC₅₀ values, a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computed area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 6
Cytotoxic potency measured by CTG assay for Mero14 cell line

| Compound | |
|---|---|
| Number | EC50 |
| 1-2 | B |
| 1-3 | A |
| 5 | A |
| 15 | A |
| 20 | A |

### Example 21—Mero82 Proliferation and Cell Viability Assay

Mero 82 cells were trypsinised, counted and plated at 400 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and absolute IC₅₀ values were calculated using GraphPad Prism software as follows: For EC₅₀ values, a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 7
Cytotoxic potency measured by CTG assay for Mero82 cell line

| Compound | |
|---|---|
| Number | EC50 |
| 1-2 | C |
| 1-3 | A |
| 5 | A |
| 15 | A |
| 20 | A |

### Example 22—BHY Proliferation and Cell Viability Assay

BHY cells were trypsinised, counted and plated at 2500 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and absolute IC₅₀ values are calculated using GraphPad Prism software as follows: For EC₅₀ values, a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 8
Cytotoxic potency measured by CTG assay for BHY cell line

| Compound | |
|---|---|
| Number | EC50 |
| 1-2 | C |
| 1-3 | A |
| 5 | B |

### Example 23—LOU-NH91 Proliferation and Cell Viability Assay

LOU-NH91 cells were trypsinised, counted and plated at 1000 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 216 hours (9 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and absolute IC₅₀ values were calculated using GraphPad Prism software as follows: For EC₅₀ values a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 9
Cytotoxic potency measured by CTG
assay for LOU-NH91 cell line

| Compound | |
|---|---|
| Number | EC50 |
| 1-2 | A |
| 1-3 | A |
| 5 | A |

### Example 24—MSTO-211H Proliferation and Cell Viability Assay

MSTO-211H cells were trypsinised, counted and plated at 1500 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment, the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and absolute IC₅₀ values were calculated using GraphPad Prism software as follows: For EC₅₀ values a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 10
Cytotoxic potency measured by CTG
assay for MSTO-211H cell line

| Compound | |
|---|---|
| Number | EC50 |
| 1-2 | A |
| 1-3 | A |
| 5 | A |
| 15 | A |
| 20 | A |

### Example 25—SDM103T2 Proliferation and Cell Viability Assay

SDM103T2 cells were trypsinised, counted and plated at 800 cells/well in 200 μl of growth media in 96 well flat bottom tissue culture treated plates. The plated cells were incubated overnight at 37° C., 5% CO₂ before being treated with compounds in triplicate for each assay point. 50 μl of the serially diluted compound was added to each well giving a final DMSO concentration of 0.5% per well and a total volume of 250 μl per well. The treated cells were incubated for 144 hours (6 days) at 37° C., 5% CO₂. Following compound treatment the remaining viable cells were detected using the Promega CellTiter-Glo 2.0 assay as follows: 150 μl of media was removed from each of the wells to be assayed and 100 μl of room temperature CellTiter-Glo 2.0 reagent was added. The plate was placed on an orbital shaker at 500 rpm at room temperature for 2 minutes, and then incubated without shaking for a further 10 minutes at room temperature. 150 μl of cell lysate was removed from each well and dispensed into a single well of a white opaque 96 well plate. Luminescence at 545 nm was read using a BMG CLARIOstar plate reader. The luminescent signal was directly proportional to number of viable cells, the percent of viable cells relative to the DMSO control was then calculated for each assay point. Percent viability relative to DMSO control was plotted against compound concentration and a curve fitted with non-linear regression in GraphPad Prism software. EC₅₀ and absolute IC₅₀ values were calculated using GraphPad Prism software as follows: For EC₅₀ values a non-constrained four-parameter logistic curve fit was used (Sigmoidal, 4PL, X is concentration). For Absolute IC₅₀ values the baseline was constrained to Y=0 and top at 100 (Absolute IC₅₀, X is concentration). Area Under the Curve (AUC): Prism computes area-under-the-curve by the trapezoidal method. The analysis sees a fitted curve as simply a series of connected XY points. Prism simply computes the area under the “curve” defined by connecting the points by straight lines. The units of the AUC are the units of the Y axis times units of the X axis.

For EC₅₀ data, the symbol “A” indicates a value of less than 0.2 μM; the symbol “B” indicates a value in the range of from 0.2 μM up to 1 μM; and the symbol “C” indicates a value of greater than 1 μM.

TABLE 11
Cytotoxic potency measured by CTG
assay for SDM103T2 cell line

| Compound | |
|---|---|
| Number | EC50 |
| 1-1 | A |
| 1-2 | C |
| 1-3 | A |
| 5 | A |
| 15 | A |
| 20 | A |

### Example 26—TEAD Thermal Shift Assay (TSA)

Thermal shift assay (TSA) is a biophysical method to evaluate stability of proteins with change in temperature. The assay measures thermal denaturation temperature in varying conditions such as different drug concentration. For this example, fluorogenic dye was used that bind to unfolded proteins. SYPRO Orange binds non-specifically to hydrophobic surfaces, and water strongly quenches its fluorescence. When the protein unfolds, the exposed hydrophobic surfaces bind the dye, resulting in an increase in fluorescence by excluding water. Detergent micelles also bind the dye and increase background noise dramatically. Binding of small molecules to proteins leads to a stabilization against to thermal denaturation with the degree of thermal stabilization proportional to the strength of binding.

The study described in this example investigated the change in melting temperature (ΔTm) of TEAD isoforms 1-4 when treated with TEAD inhibitors. Compound 1-1—a known strong TEAD1 and weak TEAD2 inhibitor; as well as Compound 1-4—a pan-TEAD inhibitor were used as controls. As expected, Compound 1-1 showed pronounced thermal shift only for TEAD1 whilst Compound 1-4 showed thermal shifts for all four TEAD isoforms. As shown in Table 12, Compound 5 displayed strong TEAD1 and TEAD4 binding.

### Experimental Parameters/Conditions

| Parameters | Conditions |
|---|---|
| Assay Format | Biophysical, 96-well Bio-Rad plate |
| Protein | Human TEADs |
| Sypro Orange | 5X Conc. |
| DMSO | 1% |
| Ramp rate | 1° C./min |
| Assay Buffer | 20 mM Tris-HCL (pH: 8), 200 mM NaCl, 2 mM TCEP, |
| | 10% Glycerol, 0.01% Tween 20 |
| Assay Volume | 25 μL |
| Assay Readout | Melt Curve 20-95° C. |
| Instrument | Bio-Rad family of CFX Real-time PCR systems |

### Assay Buffer Preparation

| Component | | | Final | | Weighed | | Total |
|---|---|---|---|---|---|---|---|
| Name | Source | Cat # | Conc. | | quantity | | Volume |
| Trizma | Sigma | T5941-500G | 20 | mM | 31.52 | mg | 10 mL |
| hydrochloride | | | | | | | |
| NaCl | Sd fine- | 20241 | 200 | mM | 116.88 | mg | |
| | chem ltd | | | | | | |
| TCEP | Sigma | C4706-2G | 2 | mM | 5.73 | mg | |

| | | | | | |
|---|---|---|---|---|---|
| Glycerol | Sigma | G5516-100ML | 10% | 1 | mL |
| Tween 20 | Sigma | P9416-100ML | 0.01% | 1 | μL |

| | | | | |
|---|---|---|---|---|
| Cell culture | Hi- | TCL019-500ML | — | — |
| grade water | media | | | |

Tris-HCL was dissolved in 5 mL of Cell culture grade water and allowed to dissolve completely. The pH was adjusted to 8 using NaOH. Additional assay buffer components were added in the order listed above. The buffer was mixed slowly.

### Assay Flow

Assay buffer was prepared according to the procedures outlined above and dispensed into test wells. Spyro orange was added to a concentration of 5× in the assay buffer, followed by test compound stock solution which was added to a concentration of 10 μM or 30 μM in the assay buffer.

The melt cycle was performed in Bio-RAD PCR instrument from 20-95° C. with ramp rate of 1° C./min.

Thermal shift data are provided in Table 12.

TABLE 12
Thermal shift assay data

| | Ratio of Protein | TEAD1 | TEAD2 | TEAD3 | TEAD4 |
|---|---|---|---|---|---|
| Compound | to Compound | ΔTm(° C.) | | | |
| 1-1 | 1:1 | 2 | 1 | 0 | 0 |
| 1-1 | 1:3 | 8 | 1 | 0 | 1 |
| 1-4 | 1:1 | 7.5 | 2.5 | 3 | 1.5 |
| 1-4 | 1:3 | 11 | 11 | 5 | 11 |
| 5 | 1:1 | 7 | 8.5 | 0 | 1.5 |
| 5 | 1:3 | 10 | 11.5 | 0 | 7 |

### Example 27—Inhibition of Mouse Xenograft MSTO-211H Tumors with TEAD Inhibitors

This study evaluated the in vivo anti-tumor efficacy of test compounds in BALB/c nude mice for treatment of MSTO-211H xenograft of lung cancer.

### Abbreviations

AAALAC Association for Assessment and Accreditation of Laboratory Animal Care
BW Body Weight
g gram
IACUC Institutional Animal Care and Use Committee
kg kilogram
mg milligram
mL milliliter
mm millimeter
PO Per Os
R&D Research and Development
RT Room Temperature
RTV Relative Tumor Volume
SEM Standard Error of the Mean
TGI Tumor Growth Inhibition
TV Tumor Volume
TW Tumor Weight
μL microliter
dd H₂O double distilled water
T/C Relative tumor proliferation rate
AA Antibiotic-Antimycotic
CMC-Na Carbokyl Methyl Cellulose-Na
BID Bis in Die
Tween 80 Tween 80

### Animals and Housing Conditions

Species: Mus Musculus
Strain: BALB/c nude
Age: 6-8 weeks
Sex: female
Body weight: 18-22 g
Number of animals inoculated with tumor: 80 mice
Number of animals enrolled in the treatment: 36 mice
Number of unenrolled animals: 44 mice
Animal supplier: Beijing Vital River Biotechnology Experimental Animal Co. Ltd Certification number: 20170011010591

### Study Design

### Part 1

| | | | | Dosing | | | |
|---|---|---|---|---|---|---|---|
| | | | Dosage | Volume^{b} | Dosing | Dosing | Dosing |
| Group | N^{a} | Compound | (mg/kg) | (μl/g) | route | Frequency^{c} | Schedule^{c} |
| 1 | 4 | Vehicle | — | 10 | PO | BID | 31 |
| 2 | 4 | 5 | 12.5 | 10 | PO | BID | 31 |
| 4 | 4 | 20 | 12.5 | 10 | PO | BID | 31 |
| 6 | 4 | 15 | 12.5 | 10 | PO | BID | 31 |

Note:
^{a}N, animal number in each group.
^{b}Dose volume: adjust dose volume based on body weight 10 μL/g.
^{c}Treatment schedule was adjusted if body weight loss >15%.

### Part 2

| | | | | Dosing | | |
|---|---|---|---|---|---|---|
| | | | Dose | Volume | Dosing | |
| Group | n | Treatment | (mg/kg) | (μl/g) | route | Schedule |
| 1 | 4 | Vehicle | — | 10 | PO | BID*30 |
| | | | | | | days |
| 3 | 4 | Compound | 12.5 | 10 | PO | BID*30 |
| | | 20 | | | | days |

### Part 3

| | | | | Dosing | | |
|---|---|---|---|---|---|---|
| | | | Dose | Volume | Dosing | |
| Group | n | Treatment | (mg/kg) | (μl/g) | route | Schedule |
| 1 | 2 | Vehicle | — | 10 | PO | BID*13 |
| | | | | | | days |
| 2 | 2 | Compound | 12.5 | 10 | PO | BID*13 |
| | | 20 | | | | days |

### Cell Culture

The MSTO-211H tumor cells were maintained in RPMI 1640 medium supplemented with 10% Fetal bovine serum and 1% Antibiotic-Antimycotic at 37° C. in an atmosphere of 5% CO₂ in air. The tumor cells were routinely subcultured twice weekly by trypsin-EDTA. The cells growing in an exponential growth phase with 95% cell survival or above were harvested, and counted for tumor inoculation.

### Tumor Cell Information

| Cell lines | Cancer Type | Species | Vendor | Cat. No. |
|---|---|---|---|---|
| MSTO-211H | Lung cancer | human | ATCC | CRL-2081 |

### Tumor Inoculation

Each mouse was inoculated subcutaneously at the right flank with MSTO-211H tumor cells (5×10⁶+50% matrigel/0.2 mL) for tumor development. Animals were marked with earrings with unique 6 significant figure number for identification. The treatments of part 1 were started on day 5 after cell inoculation when the average tumor volume reached 181 mm³. The treatments of part 2 were started on day 16 after cell inoculation when the average tumor volume reached 254 mm³. The treatments of part 3 were started on day 33 after cell inoculation when the average tumor volume reached 969 mm³.

### Tumor Measurement

The major endpoint was to determine if the tumor growth could be delayed. Tumor volume was measured twice a week in two dimensions using a caliper, and the volume was expressed in mm³ using the formula: V=0.5 a×b² where a and b are the long and short diameters of the tumor, respectively. The tumor size was then used for calculations of T/C value. The TGI value (in percent) or the T/C value (in percent) is an indication of antitumor effectiveness; T and C are the mean tumor volumes or tumor weights of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

Relative T/C (%)=T_{RTV}/C_{RTV}×100 (RTV (%)=Vₜ/V₀×100, Vₜ is the average tumor volume on a given day, V₀ is the average tumor volume on the first day of treatment. T_{RTV} is the RTV of a treatment group, and the C_{RTV} is the RTV of the vehicle control group.)

The antitumor effectiveness of part 1 study was analyzed on PG-D31 when vehicle group reached 681 mm³.

The antitumor effectiveness of part 2 study was analyzed on PG-D30 when vehicle group reached 738 mm³.

The antitumor effectiveness of part 3 study was analyzed on PG-D13 when vehicle group reached 1791 mm³.

### Statistical Analysis

Summary statistics, including mean and the standard error of the mean (SEM), were provided for the tumor volume of each group at each time point.

Statistical analysis of difference in tumor volume among the groups was conducted on the data obtained at the end of the study.

One-tailed T test was performed to compare tumor volumes between two groups. All data are analyzed using GraphPad Prism 6.0. p<0.05 is considered to be statistically significant.

### Tumor Volume Trace

The change in tumor volume over the course of the study is provided in Tables 13-15.

TABLE 13
Tumor size over course of study part 1.

| | | Days after the start of treatment | | | | | | | | | |
|---|---|---|---|---|---|---|---|---|---|---|---|
| Gr | Treatment | 0 | 3 | 6 | 10 | 13 | 17 | 20 | 23 | 27 | 31 |
| 1 | Vehicle, | 181 ± 5 | 249 ± 23 | 287 ± 32 | 327 ± 48 | 363 ± 43 | 392 ± 51 | 431 ± 51 | 489 ± 54 | 588 ± 73 | 681 ± 102 |
| | PO, BID | | | | | | | | | | |
| 2 | Compound 5, | 181 ± 11 | 241 ± 31 | 230 ± 30 | 151 ± 18 | 117 ± 10 | 63 ± 13 | 72 ± 15 | 64 ± 7 | 79 ± 13 | 81 ± 18 |
| | 12.5 mg/kg, | | | | | | | | | | |
| | PO, BID | | | | | | | | | | |
| 4 | Compound 20, | 181 ± 7 | 183 ± 26 | 188 ± 52 | 117 ± 44 | 92 ± 28 | 66 ± 10 | 60 ± 13 | 59 ± 16 | 75 ± 18 | 60 ± 11 |
| | 12.5 mg/kg, | | | | | | | | | | |
| | PO, BID | | | | | | | | | | |
| 6 | Compound 15, | 181 ± 11 | 216 ± 27 | 175 ± 19 | 154 ± 5 | 111 ± 13 | 88 ± 12 | 105 ± 6 | 134 ± 10 | 166 ± 20 | 161 ± 12 |
| | 12.5 mg/kg, | | | | | | | | | | |
| | PO, BID | | | | | | | | | | |

Note:
a. Mean ± SEM

TABLE 14
Tumor size over course of study part 2.

| | | Days after the start of treatment | | | | | | | | | |
|---|---|---|---|---|---|---|---|---|---|---|---|
| Gr. | Treatment | 0 | 2 | 6 | 9 | 12 | 16 | 20 | 23 | 27 | 30 |
| 1 | Vehicle, | 254 ± 14 | 296 ± 7 | 331 ± 9 | 367 ± 18 | 392 ± 26 | 443 ± 26 | 549 ± 71 | 579 ± 70 | 675 ± 138 | 738 ± 143 |
| | PO, BID | | | | | | | | | | |
| 3 | Compound 20, | 254 ± 30 | 249 ± 37 | 160 ± 39 | 116 ± 36 | 80 ± 13 | 75 ± 10 | 78 ± 14 | 91 ± 10 | 96 ± 6 | 164 ± 15 |
| | 12.5 mg/kg, | | | | | | | | | | |
| | PO, BID | | | | | | | | | | |

TABLE 15
Tumor size over course of study part 3.

| | | Days after the start of treatment | | | | |
|---|---|---|---|---|---|---|
| Gr. | Treatment | 0 | 3 | 6 | 10 | 13 |
| 1 | Vehicle, | 987 ± 12 | 1145 ± 31 | 1237 ± 44 | 1445 ± 138 | 1791 ± 367 |
| | PO, BID | | | | | |
| 2 | Compound 20, | 963 ± 136 | 740 ± 122 | 591 ± 61 | 444 ± 36 | 339 ± 51 |
| | 12.5 mg/kg, | | | | | |
| | PO, BID | | | | | |

### Tumor Growth Inhibition Analysis

TGI of part 1 was calculated based on tumor volume measurements on PG-D31 after the start of the treatment (Table 16).

TGI of part 2 was calculated based on tumor volume measurements on PG-D30 after the start of the treatment (Table 17).

TGI of part 3 was calculated based on tumor volume measurements on PG-D13 after the start of the treatment (Table 18).

TABLE 16
Tumor size on day 31 of study part 1.

| | | | | | P value |
|---|---|---|---|---|---|
| | | Tumor | | | compared |
| | | Volume | | | With |
| Gr. | Treatment | (mm³)^{a} | T/C^{b} (%) | TGI^{b} (%) | vehicle^{c} |
| 1 | Vehicle, | 681 ± 102 | — | — | — |
| | PO, BID | | | | |
| 2 | Compound 5, | 81 ± 18 | 11.82 | 120.17 | p < 0.001 |
| | 12.5 mg/kg, | | | | |
| | PO, BID | | | | |
| 4 | Compound 20, | 60 ± 11 | 8.81 | 124.25 | p < 0.001 |
| | 12.5 mg/kg, | | | | |
| | PO, BID | | | | |
| 6 | Compound 15, | 161 ± 12 | 23.57 | 104.12 | p < 0.001 |
| | 12.5 mg/kg, | | | | |
| | PO, BID | | | | |

^{a}Mean ± SEM.
^{b}T/C(%) = T_{RTV}/C_{RTV} × 100, TGI = [1 − (Tᵢ − T₀)/(Vᵢ − V₀)] × 100.
^{c}One-tailed T test analysis was performed to compare tumor volumes between two groups.

TABLE 17
Tumor size on day 30 of study part 2.

| | | | | | P value |
|---|---|---|---|---|---|
| | | Tumor | | | compared |
| | | Volume | | | With |
| Gr. | Treatment | (mm³) | T/C (%) | TGI (%) | vehicle^{c} |
| 1 | Vehicle, | 738 ± 143 | — | — | — |
| | PO, BID | | | | |
| 3 | Compound 20, | 164 ± 15 | 22.24 | 118.44 | p < 0.01 |
| | 12.5 mg/kg, | | | | |
| | PO, BID | | | | |

TABLE 18
Tumor size on day 13 of study part 3.

| | | | | | P value |
|---|---|---|---|---|---|
| | | Tumor | | | compared |
| | | Volume | | | With |
| Gr. | Treatment | (mm³) | T/C (%) | TGI (%) | vehicle^{c} |
| 1 | Vehicle, | 1791 ± 367 | — | — | — |
| | PO, BID | | | | |
| 2 | Compound 20, | 339 ± 51 | 18.94 | 177.56 | p < 0.05 |
| | 12.5 mg/kg, | | | | |
| | PO, BID | | | | |

### Example 28—Inhibition of Mouse Xenograft MSTO-211H Tumors with TEAD Inhibitor of the Disclosure Compared to Prior Art TEAD Inhibitor

This study evaluated the in vivo anti-tumor efficacy of test compounds in BALB/c nude mice for treatment of MSTO-211H xenograft of lung cancer.

### Study Design

| | | | | | Dosing | | |
|---|---|---|---|---|---|---|---|
| | | | Dose | | Volume | Dosing | |
| Group | n | Treatment | (mg/kg) | | (μl/g) | route | Schedule |
| 1 | 4 | Vehicle | — | | 10 | PO | BID*7 |
| | | | | | | | days |
| 2 | 2 | Compound 1-1 | 3 | mg/kg | 10 | PO | BID*7 |
| | | | | | | | days |
| 3 | 5 | Compound 5 | 12.5 | mg/kg | 10 | PO | BID*7 |
| | | | | | | | days |

The 3 mg/kg dose of Compound 1-1 was utilized based on the long half-life of Compound 1-1 as reported by Tang et al. (Tang et al., “Small Molecule Inhibitors of TEAD Auto-palmitoylation Selectively Inhibit Proliferation and Tumor Growth of NF2-deficient Mesothelioma,” Mol. Cancer Ther., 2021, 20 (6): 986-998).

### Cell Culture

The MSTO-211H tumor cells were maintained in vitro in RPMI 1640 medium supplemented with 10% Fetal bovine serum and 1% Antibiotic-Antimycotic at 37° C. in an atmosphere of 5% CO₂ in air. The tumor cells were routinely subcultured twice weekly by trypsin-EDTA. The cells growing in an exponential growth phase were harvested and counted for tumor inoculation.

### Tumor Inoculation

Each mouse was inoculated subcutaneously at the right flank with MSTO-211H tumor cells (10×10⁶+50% matrigel/0.2 mL) for tumor development. Test subjects received vehicle (e.g., 0.5% CMC-Na+1% Tween80) beginning on day 7 after cell inoculation. Treatment began on day 28 after cell inoculation when the tumor volumes reached 500 mm³ to 1200 mm³.

### Compound Preparation

| | | Purity | | | |
|---|---|---|---|---|---|
| Compounds | Preparation | (%) | Concentration | | Storage |
| 1-1 | Suspend 2.70 mg Compound 1-1 | 97.30 | 0.3 | mg/ml | 4° C. |
| | in 8.757 ml Vehicle 1, stir | | | | |
| | and sonicate to make a | | | | |
| | uniform suspension. | | | | |
| 5 | Suspend 11.25 mg Compound 5 | 96.24 | 1.25 | mg/ml | 4° C. |
| | in 8.662 ml Vehicle 1, stir | | | | |
| | and sonicate to make a | | | | |
| | uniform suspension. | | | | |

### Tumor Measurement

The major endpoint was to determine if the tumor growth could be delayed. Tumor size was measured twice a week in two dimensions using a caliper, and the volume was expressed in mm³ using the formula: V=0.5 a×b² where a and b are the long and short diameters of the tumor, respectively. The tumor size was then used for calculations of T/C value and TGI values.

The T/C value (in percent) is an indication of antitumor effectiveness; T and C are the mean volume of the treated and control groups, respectively, on a given day.

### Statistical Analysis

Summary statistics, including mean and the standard error of the mean (SEM), were calculated for the tumor volume of each group at each time point.

Statistical analysis of difference in tumor volume among the groups was conducted on the data obtained at the end of the study.

One-tailed T test was performed to compare tumor volumes between two groups. All data were analyzed using GraphPad Prism 6.0. p<0.05 was considered to be statistically significant.

### Tumor Volume Trace

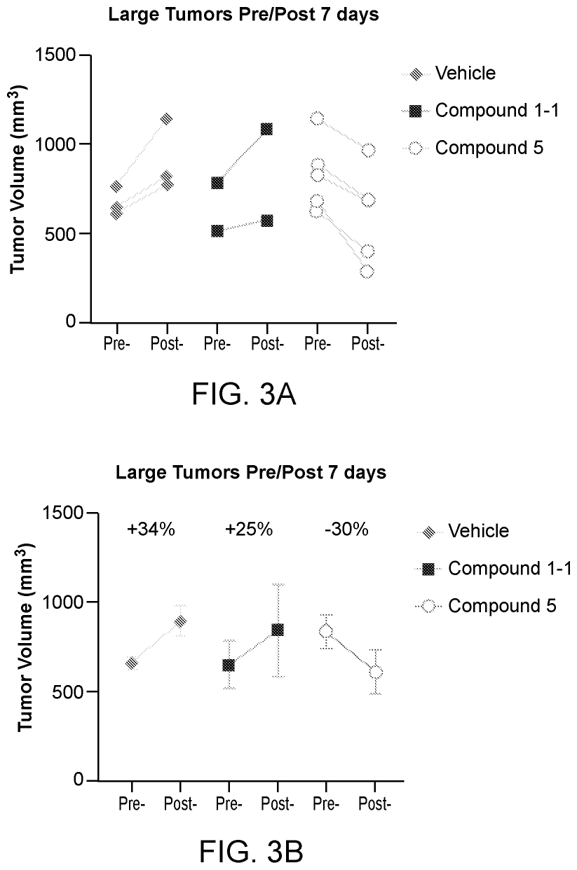

The change in tumor volume over the course of the study was recorded. The change in tumor volume after 7 days of treatment compared to pre-treatment for each of Groups 1-3 is provided in FIG. 3A and FIG. 3B. FIG. 3A provides tumor volume changes for individual test subjects, and FIG. 3B provides the mean tumor volume changes for each treatment group. Tumor growth was observed in the Vehicle and Compound 1-1 treatment groups, while tumor reduction was observed in the Compound 5 treatment group.

### Example 29—Inhibition of Mouse Xenograft NCI-11226 Tumors with TEAD Inhibitors

This study evaluated the in vivo anti-tumor efficacy of test compounds in NOD SCID mice for treatment of NCI-H226 lung tumor model.

### Abbreviations

AAALAC Association for Assessment and Accreditation of Laboratory Animal Care
BW Body Weight
g gram
IACUC Institutional Animal Care and Use Committee
kg kilogram
mg milligram
mL milliliter
mm millimeter
PO Per Os
R&D Research and Development
RT Room Temperature
RTV Relative Tumor Volume
SEM Standard Error of the Mean
TGI Tumor Growth Inhibition
TV Tumor Volume
TW Tumor Weight
μL microliter
dd H₂O double distilled water
T/C Relative tumor proliferation rate
AA Antibiotic-Antimycotic
CMC-Na Carbokyl Methyl Cellulose-Na
QD Quaque Die
BID Bis in Die
Tween 80 Tween 80

### Animals and Housing Conditions

Species: Mus Musculus
Strain: NOD SCID
Age: 6-8 weeks
Sex: female
Body weight: 18-22 g
Number of animals inoculated with tumor: 69 mice
Number of animals enrolled in the treatment: 40 mice
Number of unenrolled animals: 29 mice
Animal supplier: Beijing Vital River Biotechnology Experimental Animal Co. Ltd Certification number: 20221110Abzz0619000681

### Study Design

| | | | | Dosing | | | |
|---|---|---|---|---|---|---|---|
| | | | Dosage | Volume^{b} | Dosing | Dosing | Dosing |
| Grp | N^{a} | Compound | (mg/kg) | (μL/g) | route | Frequency^{c} | Schedule^{c} |
| 1 | 5 | No | — | | | | |
| | | Treatment | | | | | |
| 2 | 5 | Vehicle | — | 10 | PO | BID | 28 |
| 6 | 5 | 5 | 5 | 10 | PO | BID | 28 |
| 7 | 5 | 5 | 15 | 10 | PO | BID | 28 |
| 8 | 5 | 5 | 15 | 10 | PO | QD | 28 |

Note:
^{a}N, animal number in each group.
^{b}Dose volume: adjust dose volume based on body weight 10 μL/g.
^{c}Treatment schedule was adjusted if body weight loss >15%.

### Cell Culture

The NCI-H226 tumor cells were maintained in vitro in RPMI 1640 medium supplemented with 10% Fetal bovine serum and 1% Antibiotic-Antimycotic at 37° C. in an atmosphere of 5% CO₂ in air. The tumor cells were routinely subcultured twice weekly by trypsin-EDTA. The cells growing in an exponential growth phase were harvested and counted for tumor inoculation.

### Tumor Cell Information

| Cell lines | Cancer Type | Species | Vendor | Cat. No. |
|---|---|---|---|---|
| NCI-H226 | Lung cancer | human | ATCC | CRL-5826 |

### Tumor Inoculation

Each mouse was inoculated subcutaneously at the right flank with NCI-H226 tumor cells (10×10⁶+50% matrigel/0.2 mL) for tumor development. Animals were marked with earrings with unique 6 significant figure number for identification. The treatments were started when the average tumor volume reached an average size of 154 mm³.

### Tumor Measurement

The major endpoint was to determine if the tumor growth could be delayed or mice could be cured. Tumor volume was measured twice a week in two dimensions using a caliper, and the volume was expressed in mm³ using the formula: V=0.5 a×b² where a and b are the long and short diameters of the tumor, respectively. The tumor size was then used for calculations of T/C value. The TGI value (in percent) or the T/C value (in percent) is an indication of antitumor effectiveness; T and C are the mean tumor volumes or tumor weights of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on a given day, T₀ is the average tumor volume of the treatment group on the day of treatment start, Vᵢ is the average tumor volume of the vehicle control group on the same day with T₁, and V₀ is the average tumor volume of the vehicle group on the day of treatment start.

Relative T/C (%)=T_{RTV}/C_{RTV}×100 (RTV (%)=Vₜ/V₀×100, Vₜ is the average tumor volume on a given day, V₀ is the average tumor volume on the first day of treatment. T_{RTV} is the RTV of a treatment group, and the C_{RTV} is the RTV of the vehicle control group.)

Tumor weight was measured at the study termination. The T_{weight}/C_{weight} value (in percent) was calculated according to the formula: T_{weight}/C_{weight} (%)=TWₜᵣₑₐₜₘₑₙₜ/TW_{vehicle}×100, where T and C are the mean tumor weights of the treatment and control groups respectively

### Statistical Analysis

Summary statistics, including mean and the standard error of the mean (SEM), were provided for the tumor volume of each group at each time point.

Statistical analysis of difference in tumor volume among the groups was conducted on the data obtained at the end of the study.

One-tailed T test was performed to compare tumor volumes between two groups. All data were analyzed using GraphPad Prism 6.0. p<0.05 is considered to be statistically significant.

The antitumor effectiveness of the study was analyzed on PG-D28 when vehicle group reached 472 mm³.

### Tumor Volume Trace

The change in tumor volume over the course of the study is provided in Table 19.

TABLE 19
Tumor size over course of study.

| | | Days after the start of treatment^{a} | | | | | | | | |
|---|---|---|---|---|---|---|---|---|---|---|
| Gr | Treatment | 0 | 4 | 7 | 10 | 13 | 17 | 20 | 24 | 28 |
| 1 | No | 153 ± 12 | 199 ± 21 | 236 ± 24 | 267 ± 31 | 290 ± 33 | 331 ± 35 | 363 ± 35 | 422 ± 39 | 472 ± 44 |
| | Treatment | | | | | | | | | |
| 2 | Vehicle, | 153 ± 11 | 193 ± 13 | 241 ± 14 | 267 ± 14 | 293 ± 16 | 338 ± 17 | 367 ± 19 | 418 ± 18 | 476 ± 31 |
| | PO BID | | | | | | | | | |
| 6 | Compound 5, | 154 ± 13 | 140 ± 10 | 129 ± 5 | 124 ± 6 | 82 ± 8 | 77 ± 7 | 73 ± 6 | 55 ± 4 | 53 ± 5 |
| | 5 mg/kg, | | | | | | | | | |
| | PO BID | | | | | | | | | |
| 7 | Compound 5, | 154 ± 15 | 127 ± 14 | 115 ± 12 | 110 ± 10 | 84 ± 8 | 62 ± 6 | 63 ± 7 | 49 ± 4 | 43 ± 6 |
| | 15 mg/kg, | | | | | | | | | |
| | PO BID | | | | | | | | | |
| 8 | Compound 5, | 154 ± 15 | 153 ± 11 | 149 ± 11 | 148 ± 12 | 116 ± 26 | 94 ± 15 | 95 ± 16 | 85 ± 8 | 82 ± 12 |
| | 15 mg/kg, | | | | | | | | | |
| | PO QD | | | | | | | | | |

Note:
^{a}Mean ± SEM

### Tumor Growth Inhibition Analysis

Tumor growth inhibition rate for the test compounds was calculated based on tumor volume measurement at day 28 after the start of treatment. The T/C values calculated according to tumor volume are close to each other, and the trend is shown in Table 20.

TABLE 20
Tumor size on day 28.

| | | | | | P value |
|---|---|---|---|---|---|
| | | Tumor | | | compared |
| | | Volume | | | with |
| Gr. | Treatment | (mm³)^{a} | T/C^{b} (%) | TGI^{b} (%) | vehicle^{c} |
| 1 | No Treatment | 472 ± 44 | — | — | — |
| 2 | Vehicle, | 476 ± 31 | — | — | — |
| | PO BID | | | | |
| 6 | Compound 5, | 53 ± 5 | 11.2 | 131.3 | p < 0.001 |
| | 5 mg/kg, | | | | |
| | PO BID | | | | |
| 7 | Compound 5, | 43 ± 6 | 9.0 | 134.2 | p < 0.001 |
| | 15 mg/kg, | | | | |
| | PO BID | | | | |
| 8 | Compound 5, | 82 ± 12 | 17.0 | 122.3 | p < 0.001 |
| | 15 mg/kg, | | | | |
| | PO QD | | | | |

^{a}Mean ± SEM.
^{b}T/C(%) = T_{RTV}/C_{RTV} × 100, TGI = [1 − (Tᵢ − T₀)/(Vᵢ − V₀)] × 100.
^{c}One-tailed T test analysis was performed to compare tumor volumes between two groups.

What is claimed is:

1. A compound having the chemical formula (I): 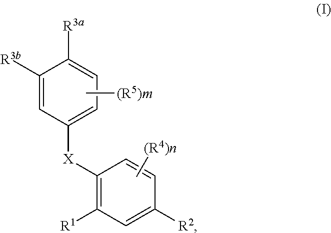
or a pharmaceutically acceptable salt thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl), wherein the 3-10 membered heterocyclyl has one, two, three, or four heteroatoms each independently selected from the group consisting of N, O, and S and is optionally substituted, wherein the C₁-C₄alkylene is optionally substituted;
one of R^{3a} and R^{3b} is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted; and the other one of R^{3a} and R^{3b} is selected from the group consisting of hydrogen, halogen, and —C₁-C₆ alkyl;
each R⁴ is independently selected from halogen and —C₁-C₆ alkyl;
each R⁵ is independently selected from halogen and —C₁-C₆ alkyl;
m is selected from the group consisting of 0, 1, 2, and 3;
n is selected from the group consisting of 0, 1, 2, and 3; and
R^{a} is selected from hydrogen and —C₁-C₆ alkyl.

2. A compound having the chemical formula (I′): 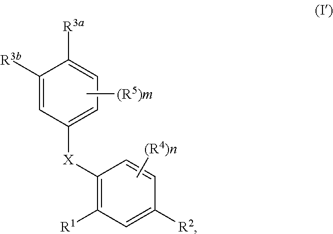
or a pharmaceutically acceptable salt thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl), wherein the 3-10 membered heterocyclyl contains at least 2 carbon atoms and one, two, three or, four heteroatoms and is optionally substituted, wherein the heteroatoms are nitrogen, and wherein the C₁-C₄alkylene is optionally substituted;
one of R^{3a} and R^{3b} is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted; and the other one of R^{3a} and R^{3b} is selected from the group consisting of hydrogen, halogen, and —C₁-C₆ alkyl;
each R⁴ is independently selected from halogen and —C₁-C₆ alkyl;
each R⁵ is independently selected from halogen and —C₁-C₆ alkyl;
m is selected from the group consisting of 0, 1, 2, and 3;
n is selected from the group consisting of 0, 1, 2, and 3; and
R^{a} is selected from hydrogen and —C₁-C₆ alkyl.

3. A compound having the chemical formula (I-1): 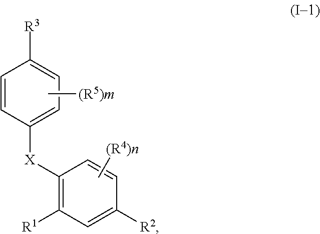
or a pharmaceutically acceptable salt thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl); wherein the 3-10 membered heterocyclyl has one, two, three, or four heteroatoms each independently selected from the group consisting of N, O, and S, and is optionally substituted, wherein the C₁-C₄alkylene is optionally substituted;
R³ is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted;
each R⁴ is independently selected from halogen and —C₁-C₆ alkyl;
each R⁵ is independently selected from halogen and —C₁-C₆ alkyl;
m is selected from the group consisting of 0, 1, 2, 3, and 4;
n is selected from the group consisting of 0, 1, 2, and 3; and
R^{a} is selected from hydrogen and —C₁-C₆ alkyl.

4. A compound having the chemical formula (I-1′): 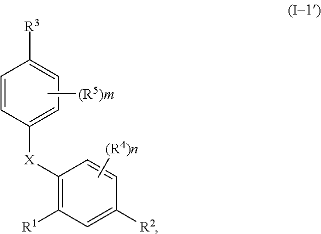
or a pharmaceutically acceptable salt thereof, wherein:
X is selected from the group consisting of NR^{a}, O, and S;
R¹ is a 5-10 membered heteroaryl having one, two, three, or four heteroatoms each independently selected from the group consisting of N, O, and S; wherein the 5-10 membered heteroaryl is optionally substituted;
R² is 3-10 membered heterocyclyl or —(C₁-C₄alkylene)-(3-10 membered heterocyclyl);
wherein the 3-10 membered heterocyclyl contains at least 2 carbon atoms and one, two, three, or four heteroatoms and is optionally substituted, wherein the heteroatoms are nitrogen, and wherein the C₁-C₄alkylene is optionally substituted;
R³ is selected from the group consisting of halogen, —C₁-C₆ alkyl, —C₁-C₆ haloalkyl, —O—(C₁-C₆ alkyl), —O—(C₁-C₆ haloalkyl), C₃₋₁₀ cycloalkyl, phenyl, naphthyl, and 5-10 membered heteroaryl, wherein the C₃₋₁₀ cycloalkyl, phenyl, naphthyl, and 5-10 membered heteroaryl are optionally substituted;
each R⁴ is independently selected from halogen and —C₁-C₆ alkyl;
each R⁵ is independently selected from halogen and —C₁-C₆ alkyl;
m is selected from the group consisting of 0, 1, 2, 3, and 4;
n is selected from the group consisting of 0, 1, 2, and 3; and
R^{a} is selected from hydrogen and —C₁-C₆ alkyl.

5. The compound of any one of claims 1-4, wherein X is NR^{a}.

6. The compound of claim 1 or 3, wherein the compound is a compound of formula (Ia): 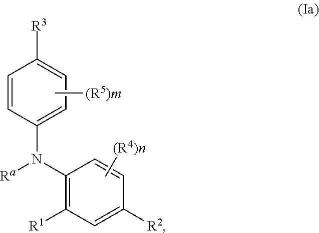
or a pharmaceutically acceptable salt thereof, wherein:
R³ is —C₁-C₆ alkyl or —C₁-C₆ haloalkyl; and
the rest of the variables are as defined in claim 1.

7. The compound of claim 2 or 4, wherein the compound is a compound of formula (Ia′): 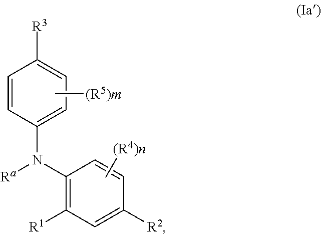
or a pharmaceutically acceptable salt thereof, wherein:
R³ is —C₁-C₆ alkyl or —C₁-C₆ haloalkyl; and
the rest of the variables are as defined in claim 2.

8. The compound of any one of claims 1-7, wherein R¹ is 5-6 membered heteroaryl that is optionally substituted.

9. The compound of any one of claims 1-8, wherein R¹ is 5-6 membered heteroaryl that is optionally substituted with 1, 2, 3, or 4 substituents each independently selected from the group consisting of C₁-C₆ alkyl, halogen, C₁-C₆ haloalkyl, —O—(C₁-C₆ alkyl), —O—(C₁-C₆ haloalkyl), and —N(R^{b})₂, wherein each R^{b} is independently selected from hydrogen and C₁-C₆ alkyl.

10. The compound of any one of claims 1-9, wherein R¹ is 5-6 membered heteroaryl that is optionally substituted with 1, 2, 3, or 4 substituents each independently selected from halogen and —C₁-C₆ alkyl.

11. The compound of any one of claims 1-10, wherein R¹ is 5-6 membered heteroaryl selected from the group consisting of oxadiazole, imidazole, pyrazole, oxazole, triazole, tetrazole, pyridine, pyrazine, and pyridazine, wherein the 5-6 membered heteroaryl is optionally substituted with 1, 2, 3, or 4 substituents each independently selected from the group consisting of C₁-C₆ alkyl, halogen, C₁-C₆ haloalkyl, —O—(C₁-C₆ alkyl), —O—(C₁-C₆ haloalkyl), and —N(R^{b})₂, wherein each R^{b} is independently selected from hydrogen and C₁-C₆ alkyl.

12. The compound of any one of claims 1-11, wherein R¹ is 5-6 membered heteroaryl selected from the group consisting of oxadiazole, imidazole, pyrazole, oxazole, triazole, tetrazole, pyridine, pyrazine, and pyridazine, wherein the 5-6 membered heteroaryl is optionally substituted with C₁-C₆ alkyl.

13. The compound of any one of claims 1-12, wherein R¹ is imidazole optionally substituted with methyl.

14. The compound of any one of claims 1-12, wherein R¹ is oxazole.

15. The compound of any one of claims 1-12, wherein R¹ is tetrazole optionally substituted with methyl.

16. The compound of any one of claims 1-12, wherein R¹ is oxadiazole, e.g., 1,3,4-oxadiazole, optionally substituted with methyl.

17. The compound of any one of claims 1, 3, and 6, wherein the compound is a compound of formula (Ib): 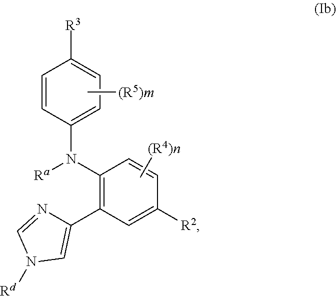
or a pharmaceutically acceptable salt thereof, wherein:
R^{d} is selected from hydrogen and —C₁-C₆ alkyl; and
the rest of the variables are as defined in claim 6.

18. The compound of any one of claims 2, 4, and 7, wherein the compound is a compound of formula (Ib′): 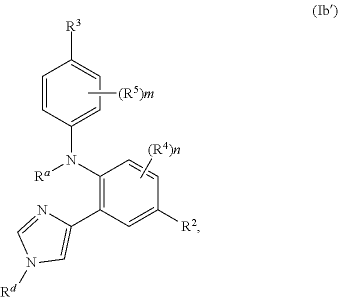
or a pharmaceutically acceptable salt thereof, wherein:
R^{d} is selected from hydrogen and —C₁-C₆ alkyl; and
the rest of the variables are as defined in claim 7.

19. The compound of claim 17, wherein the compound is a compound of formula (Ic): 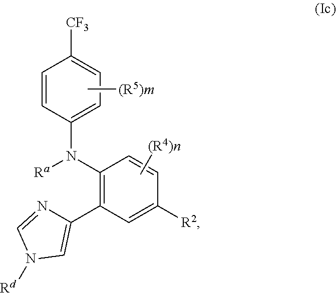
or a pharmaceutically acceptable salt thereof, wherein the variables are as defined in claim 17.

20. The compound of claim 18, wherein the compound is a compound of formula (Ic′): 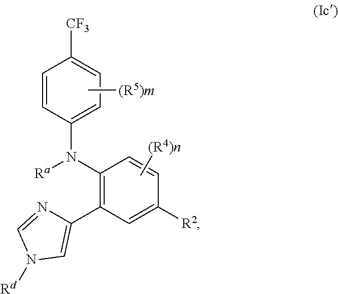
or a pharmaceutically acceptable salt thereof, wherein the variables are as defined in claim 18.

21. The compound of any one of claims 1-20, wherein R² is 3-7 membered monocyclic heterocyclyl or —(C₁-C₄alkylene)-(3-7 membered monocyclic heterocyclyl), wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted, and wherein the C₁-C₄alkylene is optionally substituted (e.g., optionally substituted with C₁₋₆alkyl, C₁₋₆haloalkyl, or two geminal hydrogens on a carbon of the C₁₋₄alkylene can be taken together with the carbon atom to which they are attached to form a 3-7 membered cycloalkyl ring).

22. The compound of any one of claims 1-21, wherein R² is 3-7 membered monocyclic heterocyclyl or —(C₁-C₄alkylene)-(3-7 membered monocyclic heterocyclyl), wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted with 1, 2, 3, or 4 substituents each independently selected from the group consisting of C₁-C₆ alkyl, oxo, hydroxyl, halogen, C₁-C₆ haloalkyl, —O—(C₁-C₆ alkyl), —O—(C₁-C₆ haloalkyl), —N(R^{b})₂, —C(O)OR^{g}, —C(O)N(R^{f})₂, —S(O)₂N(R^{f})₂, —OC(O)N(R^{f})₂, —NR^{f}C(O)N(R^{f})₂, and —S(O)ᵣ—R^{f}, wherein each of R^{b}, R^{g}, and R^{f} is independently selected from hydrogen and C₁-C₆ alkyl, wherein each r is 1 or 2, and wherein each R^{f} is independently selected from C₁₋₆alkyl and phenyl.

23. The compound of any one of claims 1-22, wherein R² is 3-7 membered monocyclic heterocyclyl, wherein the 3-7 membered monocyclic heterocyclyl contains at least 2 carbon atoms and one, two, three, or four heteroatoms, wherein the heteroatoms are nitrogen and wherein the 3-7 membered monocyclic heterocyclyl is optionally substituted with 1, 2, 3, or 4 substituents each independently selected from the group consisting of halogen, —C₁-C₆ alkyl, oxo, and hydroxyl.

24. The compound of any one of claims 1-22, wherein R² is 5-membered heterocyclyl having one or two heteroatoms, wherein the heteroatoms are nitrogen atoms, and wherein the 5-membered heterocyclyl is optionally substituted with 1, 2, or 3 substituents each independently selected from C₁-C₆ alkyl, oxo, and hydroxyl.

25. The compound of any one of claims 1-22, wherein R² is —(C₁-C₄alkylene)-(5-membered heterocyclyl having one or two heteroatoms), wherein the heteroatoms are nitrogen atoms, and wherein the 5-membered heterocyclyl is optionally substituted with 1, 2, or 3 substituents each independently selected from C₁-C₆ alkyl oxo, and hydroxyl.

26. The compound of any one of claims 1-22, wherein R² is: 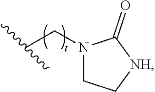
wherein t is selected from the group consisting of 0, 1, 2, and 3.

27. The compound of any one of claims 1-22, wherein R² is: 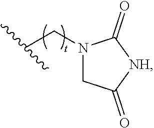
wherein t is selected from the group consisting of 0, 1, 2, and 3.

28. The compound of claim 26 or 27, wherein t is 1.

29. The compound of any one of claims 1-22, wherein R² is: 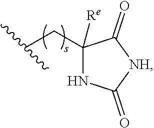
wherein s is selected from the group consisting of 0, 1, 2, and 3, and R^{e} is selected from hydrogen and C₁-C₆ alkyl.

30. The compound of claim 29, wherein s is 0 and R^{e} is methyl.

31. The compound of any one of claim 1-22, wherein R² is: 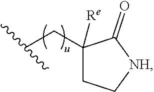
wherein u is selected from the group consisting of 0, 1, 2, and 3, and R^{e} is selected from hydrogen and C₁-C₆ alkyl.

32. The compound of any one of claims 1-22, wherein R² is: 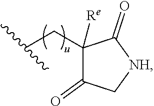
wherein u is selected from the group consisting of 0, 1, 2, and 3, and R^{e} is selected from hydrogen and C₁-C₆ alkyl.

33. The compound of any one of claims 1-22, wherein R² is: 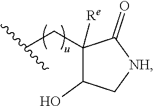
wherein u is selected from the group consisting of 0, 1, 2, and 3, and R^{e} is selected from hydrogen and C₁-C₆ alkyl.

34. The compound of any one of claims 31-33, wherein u is 0 and R^{e} is methyl.

35. The compound of any one of claims 1-24 and 31, wherein the compound is a compound of formula (Id): 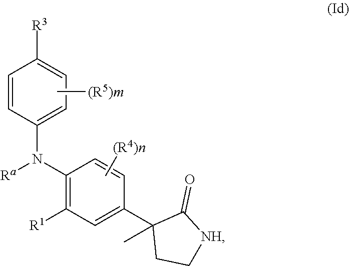
or a pharmaceutically acceptable salt thereof,
wherein R³ is —C₁-C₆ alkyl or —C₁-C₆ haloalkyl; and
the rest of the variables are as defined in claim 6.

36. The compound of claim 35, wherein the compound is a compound of formula (Je): 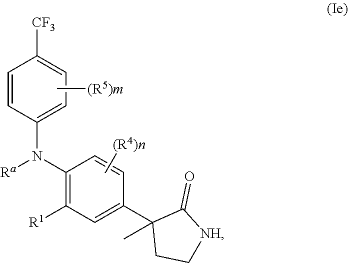
or a pharmaceutically acceptable salt thereof, wherein the variables are as defined in claim 35.

37. The compound of any one of claims 1-10 and 21-36, wherein R¹ contains at least one ring carbon atom and contains only carbon and nitrogen atoms.

38. The compound of any one of claims 1-37, wherein R³ is C₁-C₆ alkyl or —C₁-C₆ haloalkyl.

39. The compound of any one of claims 1-38, wherein R³ is halogen.

40. The compound of any one of claims 1-39, wherein R³ is trifluoromethyl.

41. The compound of any one of claims 1-40, wherein m and n are both 0.

42. The compound of any one of claims 1-41, wherein R^{a} is hydrogen.

43. The compound of any one of claims 1-42, wherein the compound is selected from Table 1 or a pharmaceutically acceptable salt thereof.

44. A pharmaceutical composition, comprising: (a) a compound or a pharmaceutically acceptable salt thereof of any one of claims 1-43; and (b) a pharmaceutically acceptable excipient.

45. A method of treating a disease or condition mediated by hyperactivation of a TEAD isoform selected from TEAD1 and TEAD4 in a subject in need thereof, comprising: administering to the subject a therapeutically-effective amount of a compound or pharmaceutically acceptable salt of any one of claims 1-43, or a pharmaceutical composition of claim 44.

46. The method of claim 45, wherein the disease or condition is a cancer characterized by hyperactivation of a TEAD isoform selected from TEAD1 and TEAD4.

47. The method of claim 46, wherein the cancer is selected from the group consisting of: a breast cancer, a lung cancer, a gastric cancer, a colorectal cancer, a pancreatic cancer including pancreatic adenocarcinoma, a mesothelioma including malignant mesothelioma, a hepatocellular cancer, a prostate cancer, a head and neck cancer, a renal cell carcinoma, and a medulloblastoma.

48. The method of claim 47, wherein the pancreatic cancer is pancreatic adenocarcinoma.

49. The method of claim 47, wherein the mesothelioma is malignant mesothelioma.

50. The method of 46 or 47, wherein the cancer is selected from the group consisting of: a hepatocellular cancer, a breast cancer, a pancreatic adenocarcinoma, and a mesothelioma.

51. The method of any one of claims 46, 47, and 50, wherein the cancer is mesothelioma.

52. The method of any one of claims 46-51, wherein the cancer is a metastatic cancer.

53. The method of claim 52, wherein the cancer is a metastatic breast cancer.

54. The method of claim 52, wherein the cancer is a metastatic lung cancer.

55. The method of claim 52, wherein the cancer is a metastatic gastric cancer.

56. The method of claim 52, wherein the cancer is a metastatic colorectal cancer.

57. The method of claim 52, wherein the cancer is a metastatic prostate cancer.

58. The method of claim 52, wherein the cancer is a metastatic head and neck cancer.

59. The method of claim 52, wherein the cancer is a metastatic renal cell carcinoma.

60. The method of claim 52, wherein the cancer is a metastatic mesothelioma.

61. The method of claim 52, wherein the cancer is a metastatic pancreatic cancer.

62. The method of claim 52, wherein the cancer is a metastatic hepatocellular cancer.

63. The method of any one of claims 45-62, wherein the therapeutically-effective amount of a compound or pharmaceutically acceptable salt of any one of claims 1-43, or the pharmaceutical composition of claim 44, is administered in combination with one or more other therapeutic agents.

64. A compound or pharmaceutically acceptable salt of any one of claims 1-43, or the pharmaceutical composition of claim 44, for use in treating a disease or condition mediated by hyperactivation of a TEAD isoform selected from TEAD1 and TEAD4 in a subject in need thereof.

65. The compound or pharmaceutically acceptable salt or pharmaceutical composition for use of claim 64, wherein the disease or condition is a cancer characterized by hyperactivation of a TEAD isoform selected from TEAD1 and TEAD4.

66. The compound or pharmaceutically acceptable salt or pharmaceutical composition for use of claim 65, wherein the cancer is selected from the group consisting of: a breast cancer, a lung cancer, a gastric cancer, a colorectal cancer, a pancreatic cancer including pancreatic adenocarcinoma, a mesothelioma including malignant mesothelioma, a hepatocellular cancer, a prostate cancer, a head and neck cancer, a renal cell carcinoma, and a medulloblastoma.

67. Use of a compound or pharmaceutically acceptable salt of any one of claims 1-43, or a pharmaceutical composition of claim 44 in the manufacture of a medicament for treating a disease or condition mediated by hyperactivation of a TEAD isoform selected from TEAD1 and TEAD4 in a subject in need thereof.

68. The use of claim 67, wherein the disease or condition is a cancer characterized by hyperactivation of a TEAD isoform selected from TEAD1 and TEAD4.

69. The use of claim 68, wherein the cancer is selected from the group consisting of: a breast cancer, a lung cancer, a gastric cancer, a colorectal cancer, a pancreatic cancer including pancreatic adenocarcinoma, a mesothelioma including malignant mesothelioma, a hepatocellular cancer, a prostate cancer, a head and neck cancer, a renal cell carcinoma, and a medulloblastoma.
